(19) United States
(12) Patent Application Publication
(10) Pub. No.: US 2022/0073822 A1
(43) Pub. Date: Mar. 10, 2022

(54) LIQUID-CRYSTAL MEDIUM

(71) Applicant: Merck Patent GmbH, Darmstat (DE)

(72) Inventors: Dagmar KLASS, Darmstadt (DE); Carsten FRITZSCH, Darmstadt (DE); Constanze BROCKE, Gross-Gerau (DE); Dmitry USHAKOV, Muenster (DE); Beate SCHNEIDER, Seeheim-Jugenheim (DE)

(73) Assignee: Merck Patent GmbH, Darmstadt (DE)

(21) Appl. No.: 17/312,996

(22) PCT Filed: Dec. 11, 2019
(86) PCT No.: PCT/EP2019/084672

§ 371 Date: Jun. 11, 2021

(30) Foreign Application Priority Data
Dec. 13, 2018 (EP) 18212251.5

(51) Int. Cl. C09K 19/34; C09K 19/18; C09K 19/44; C09K 19/16; H01Q 3/26

(52) U.S. Cl. CPC C09K 19/3491; C09K 19/18; C09K 2019/123; C09K 19/16; H01Q 3/2676; C09K 19/44

(57) ABSTRACT

The present invention relates to components for high-frequency technology, especially microwave components for high-frequency devices, such as devices for shifting the phase of microwaves, tunable filters, tunable metamaterial structures and electronic beam steering antennas (e.g. phased array antennas) comprising a liquid-crystalline medium, and to the liquid-crystalline medium used therein, wherein the liquid crystal medium comprises one or more compounds of formula S

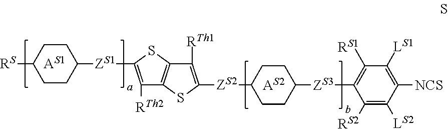

as defined in claim 1.

The present invention relates to a liquid-crystal medium, to components for high-frequency technology comprising said medium, especially microwave components for high-frequency devices, such as devices for shifting the phase of microwaves, tunable filters, tunable metamaterial structures and electronic beam steering antennas (e.g. phased array antennas.

Liquid-crystalline media have been used for many years in electro-optical displays (liquid crystal displays: LCDs) in order to display information. More recently, liquid-crystalline media have also been proposed for use in components for microwave technology, such as, for example, in DE 10 2004 029 429 A and in JP 2005-120208 (A).

DE 10 2004 029 429 A describes the use of liquid-crystal media in microwave technology, inter alia in phase shifters. Therein, liquid-crystalline media with respect to their properties in the corresponding frequency range have been discussed and liquid-crystalline media based on mixtures of mostly aromatic nitriles and isothiocyanates have been shown. Isothiocyanates derived from thieno[3,2-b]thiophene are proposed for the use in liquid-crystal light modulators in CN 106518890 A. In EP 2 982 730 A1, LC mixtures are shown that consist of isothiocyanate compounds. However, these compositions are all still afflicted with several disadvantages as for example high dielectric loss or inadequate phase shift or inadequate material quality, resulting in limited performance in devices operating in the microwave regime. Further it is required to improve these media with respect to their general physical properties, such as, the clearing point, the phase range, especially their storage stability at low temperatures, and their viscosities, in particular their rotational viscosity.

There is still a demand for devices for high frequency-technology comprising LC media with improved performance.

For these applications, liquid-crystalline media having particular, hitherto rather unusual and uncommon properties or combinations of properties are required. One aspect of the present invention is to provide LC media with properties that enable improved devices for microwave technology. It has been recognised that the dielectric loss in the microwave region can be reduced and the material quality (η, also known as “figure of merit” (FoM) i.e., a high tunability and a low dielectric loss) can be improved. Besides these requirements the focus must increasingly be directed to improved response times especially for those devices using planar structures such as e.g. phase shifters and leaky antennas.

In addition, it has been recognised that an improvement in the low-temperature behaviour of the components results in an improvement in the operating properties at low temperatures and also in the shelf life. Especially upon cooling, the formation of smectic phases or crystallisation is undesired and may even lead to the destruction of a device. The existence of a nematic phase without formation of smectic phases or crystallisation at low temperatures and over a period of time sufficient for the operation of a device is referred to as low temperature stability (LTS).

Therefore, there is a considerable demand for liquid-crystalline media having suitable properties for corresponding practical applications.

Surprisingly, it has been found that it is possible to achieve components for high-frequency technology with improved performance by using liquid-crystalline media that comprise a thienothiophene derivative of formula S below.

The present invention relates to a liquid crystal medium for use in a component operable in the microwave region of the electromagnetic spectrum, characterised in that the medium comprises one, two or more compounds of formula S

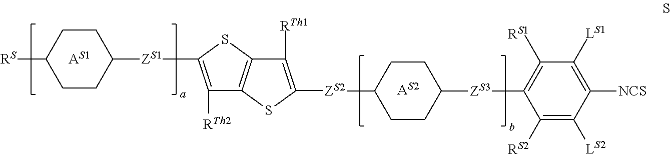

in which
R^{S} denotes H, alkyl or alkoxy having 1 to 12 C atoms, or alkenyl, alkenyloxy or alkoxyalkyl having 2 to 12 C atoms, in which one or more CH₂-groups may be replaced by

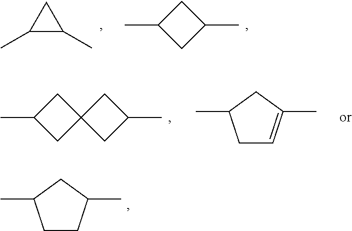

and in which one or more H atoms may be replaced by F,

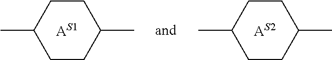

on each occurrence, independently of one another, denote

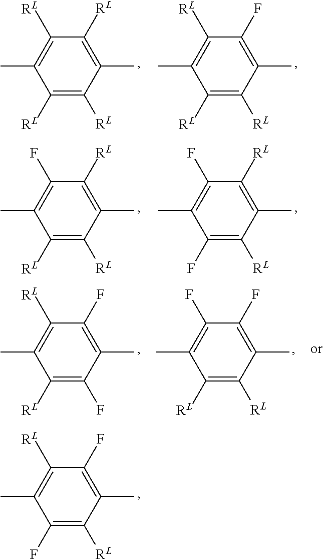

in which R^{L}, on each occurrence identically or differently, denotes H, Cl or straight chain, branched or cyclic alkyl having 1 to 6 C atoms,

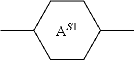

alternatively denotes

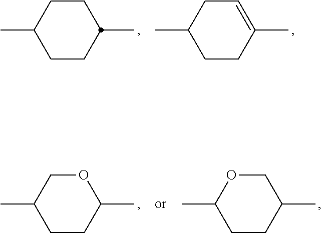

L^{S1}, L^{S2} identically or differently, denote H, Cl or F,
R^{S1}, R^{S2}, identically or differently, denote H, alkyl or alkenyl, having up to 6 C atoms, or cyclopropyl, cyclobutyl, cyclopentenyl, or cyclopentyl,
R^{Th1}, R^{Th2} identically or differently, denote H, alkyl or alkenyl or alkoxy, having up to 6 C atoms, or cyclopropyl, cyclobutyl, cyclopentenyl or cyclopentyl,
Z^{S1}, Z^{S2}, Z^{S3} identically or differently, denote —CH═CH—, —CH═CF—, —CF═CH—, —CF═CF—, —C≡C—, or a single bond,
a, b identically or differently, are 0 or 1,

and

optionally, one or more compounds of formula IC

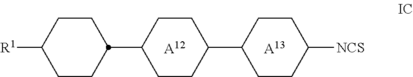

in which
R¹ denotes H, alkyl or alkoxy having 1 to 15 C atoms, or alkenyl, alkenyloxy or alkoxyalkyl having 2 to 15 C atoms, in which one or more CH₂-groups may be replaced by

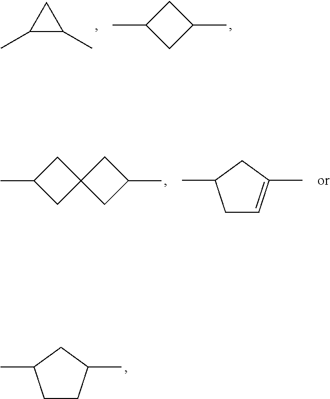

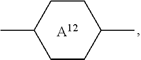

, on each occurrence, independently of one another, denote

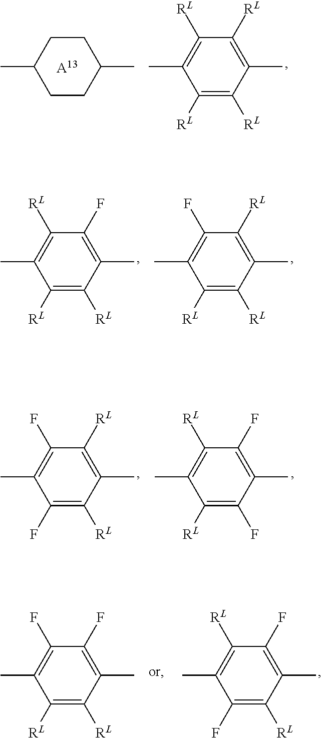

in which RH, on each occurrence identically or differently, denotes H, Cl or straight chain, branched or cyclic alkyl having 1 to 6 C atoms.

A first aspect of the present invention relates to LC media comprising a compound of formula S and a compound of formula IC.

The present invention further relates to a compound of formula S as defined above, with the proviso that the compounds of formula S* are excluded:

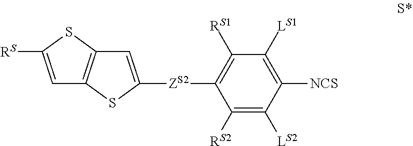

in which
R^{S} denotes alkyl, alkenyl or alkoxy having up to 9 C atoms,
Z^{S2} denotes —CH═CH—, —CH═CF—, —CF═CH—, —CF═CF—, —C≡C—, or a single bond,
R^{S1}, R^{S2} identically or differently, denote H or F, and

L^{S1}, L^{S2} identically or differently, denote H or F.

Another aspect of the present invention relates to liquid crystal media comprising one or more compounds of formula S wherein compounds of formula S* defined above are excluded.

The present invention further relates to a component operable in the microwave region of the electromagnetic spectrum comprising the liquid crystal medium according to the invention.

Components for high frequency technology that make use of a liquid-crystalline medium as a switchable dielectric which comprises one or more compounds of formula S are distinguished by fast switching times, a broad operating temperature range, high tunability and low dielectric loss.

A further object of the present invention are devices for microwave technology comprising said components.

Preferred components are phase shifters, varactors, wireless and radio wave antenna arrays, matching circuits, adaptive filters and others.

The media according to the present invention are distinguished by a particularly high birefringence, furthermore a high clearing temperature, excellent low-temperature stability and broad nematic phase range. As a result, devices according to the invention containing the media are operable under extreme temperature conditions.

The media are further distinguished by high values of the dielectric anisotropy and a low rotational viscosities. As a result, the threshold voltage, i.e. the minimum voltage at which a device is switchable, is very low. A low operating voltage and low threshold voltage is desired in order to enable a device having improved switching characteristics and high energy efficiency. Low rotational viscosities enable fast switching of the devices according to the invention.

The media according to the present invention are distinguished by low dielectric loss and high tunability, resulting in a high material quality (η).

The compounds of formula S are distinguished by an advantageously high birefringence, high solubility in liquid crystalline media, high tunability and low dielectric loss.

These properties as a whole make the media particularly suitable for use in components and devices for high-frequency technology and applications in the microwave range, in particular devices for shifting the phase of microwaves, tunable filters, tunable metamaterial structures, and electronic beam steering antennas (e.g. phased array antennas). Herein, halogen is F, Cl, Br or I, preferably F or Cl, particularly preferably F.

Herein, an alkyl radical and/or an alkoxy radical, can be straight-chain or branched. It is preferably straight-chain, has 2, 3, 4, 5, 6 or 7 carbon atoms and accordingly is preferably ethyl, propyl, butyl, pentyl, hexyl, heptyl, ethoxy, propoxy, butoxy, pentoxy, hexoxy or heptoxy, furthermore methyl, octyl, nonyl, decyl, undecyl, dodecyl, tridecyl, tetradecyl, pentadecyl, methoxy, octoxy, nonoxy, decoxy, undecoxy, dodecoxy, tridecoxy or tetradecoxy. Branched alkyl is preferably isopropyl, isobutyl, sec.-butyl, tert.-butyl, 2,2-dimethylpropyl, 3-methylbutyl, 1-methylbutyl, 1-ethylpropyl, 1,2-dimethylpropy, 2-methylbutyl. Cyclic alkyl is preferably cyclopropyl, cyclobutyl, cyclopentyl, and cyclohexyl, all of which can be substituted by one or more, preferably one, alkyl group, preferably by methyl or ethyl.

Herein, an alkenyl radical may have from 2 to 15 carbon atoms, which may be straight-chain or branched. It is preferably straight-chain and has from 2 to 7 carbon atoms. Accordingly, it is preferably vinyl, prop-1- or -2-enyl, but-1-, -2- or -3-enyl, pent-1-, -2-, -3- or -4-enyl, hex-1-, -2-, -3-, -4- or -5-enyl, or hept-1-, -2-, -3-, -4-, -5- or -6-enyl.

Herein, oxaalkyl is preferably straight-chain 2-oxapropyl (=methoxymethyl), 2-oxabutyl (=ethoxymethyl) or 3-oxabutyl (=methoxyethyl), 2-, 3- or 4-oxapentyl, 2-, 3-, 4- or 5-oxahexyl, or 2-, 3-, 4-, 5- or 6-oxaheptyl.

Herein, in an alkyl radical having from 1 to 15 carbon atoms in which one CH₂ group has been replaced by —O— and one has been replaced by —CO—, these are preferably adjacent. This thus contains an acyloxy group —CO—O— or an oxycarbonyl group —O—CO—. This is preferably straight-chain and has from 2 to 6 carbon atoms.

Herein, alkyl radical having from 1 to 15 carbon atoms in which one CH₂ group has been replaced by unsubstituted or substituted —CH═CH— and an adjacent CH₂ group has been replaced by CO or CO—O or O—CO, where this may be straight-chain or branched is preferably straight-chain and has from 4 to 13 carbon atoms.

Herein, an alkyl radical having from 1 to 15 carbon atoms or an alkenyl radical having from 2 to 15 carbon atoms, each of which is at least monosubstituted by halogen (F, Cl, Br, I) are preferably straight-chain and halogen is preferably —F or —Cl. In the case of polysubstitution, halogen is preferably —F. The resultant radicals also include perfluorinated radicals, such as —CF₃. In the case of monosubstitution, the fluorine or chlorine substituent can be in any desired position.

Herein, an alkyl or alkoxy radical having 1 to 15 C atoms, preferably 1 to 5, particularly preferably 1, where one or more CH₂ groups, preferably one, in these radicals may each be replaced, independently of one another, by

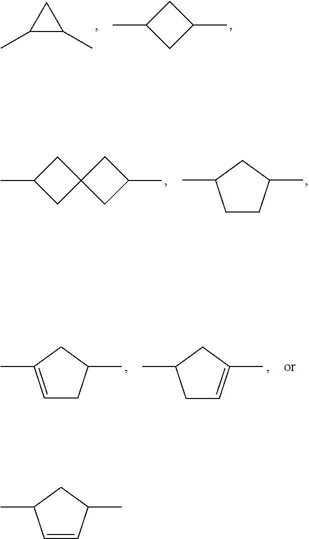

is preferably cyclopropyl, cyclobutyl, cyclopentyl or cyclopent-1-enyl, or cyclopropylmethyl.

In case R^{F} denotes a halogenated, preferably fluorinated alkyl-, alkoxy-, alkenyl or alkenyloxy it can be branched or unbranched. Preferably it is unbranched and has 1, 2, 3, 4, 5, 6 or 7 C atoms, in case of alkenyl 2, 3, 4, 5, 6 or 7 C atoms. It can be partially fluorinated or perfluorinated, preferably perfluorinated.

R^{T} preferably denotes CN, NCS, Cl, F, —(CH₂)ₙ—CH═CF₂, —(CH₂)ₙ—CH═CHF, —(CH₂)ₙ—CH═Cl₂, —CₙF₂ₙ₊₁, —(CF₂)ₙ—CF₂H, —(CH₂)ₙ—CF₃, —(CH₂)ₙ—CHF₂, —(CH₂)ₙCH₂F, —CH═CF₂, —O(CH₂)ₙ—CH═CF₂, —O(CH₂)ₙCHCl₂, —OCₙF₂ₙ₊₁, —O(CF₂)ₙ—CF₂H, —O(CH₂)ₙCF₃, —O(CH₂)ₙ—CHF₂, —O(CF)ₙCH₂F, —OCF═CF₂, —SCₙF₂ₙ₊₁, —S(CF)ₙ—CF₃, wherein n is an integer from 0 to 7.

Preferably, the compounds of formula S are selected from the group of compounds of the formulae S-1 to S-24:

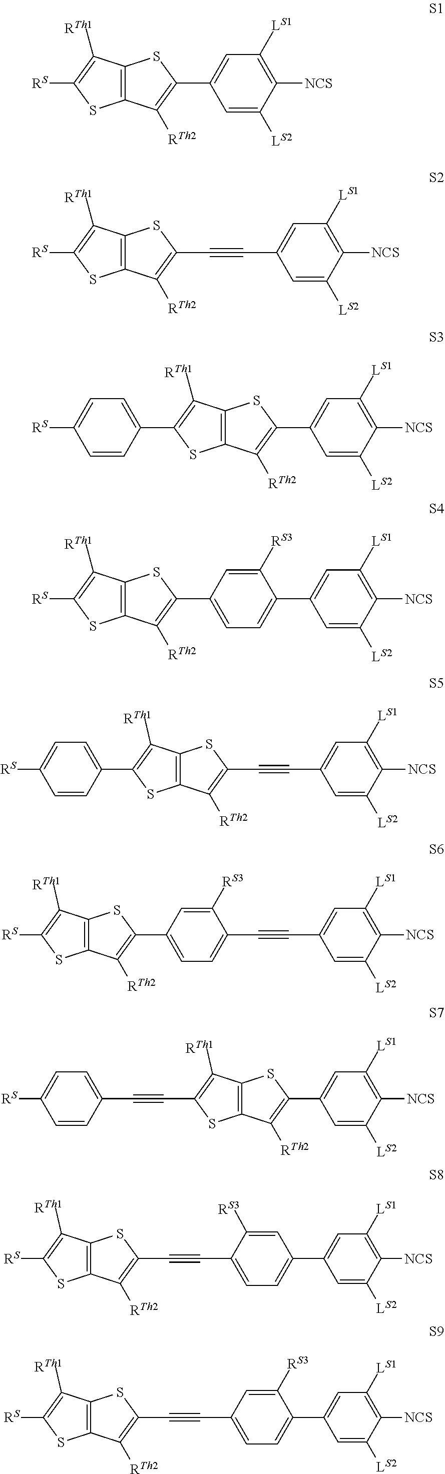

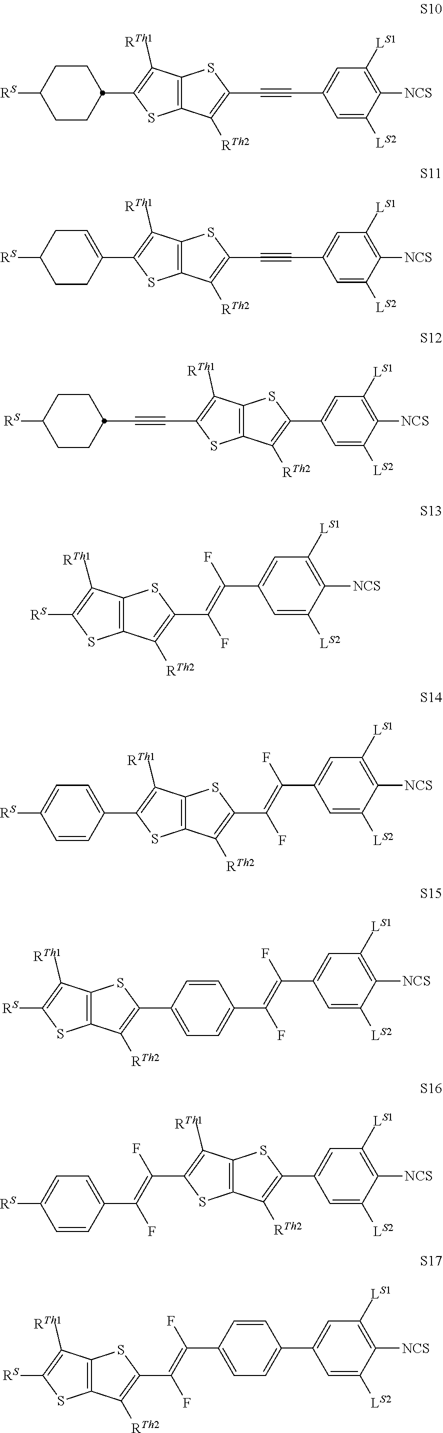

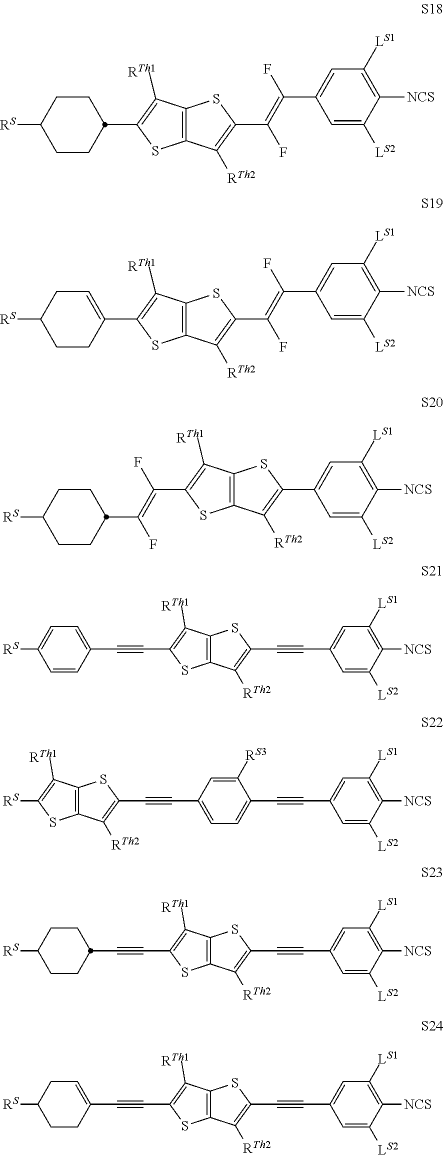

in which R^{S3} denotes F or has the meaning of R^{L} defined above and the other occurring groups have the meanings given above for formula S and preferably
R^{S} denotes alkyl or alkenyl having 2 to 6 C atoms, in which one or more CH₂-groups may be replaced by

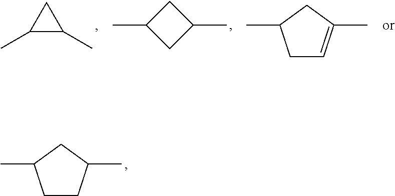

R^{Th1} and
R^{Th2} identically or differently, denote H or alkyl having 1 to 6 C atoms, preferably H,
R^{S3} denotes H, F or alkyl, having up to 6 C atoms, or cyclopropyl, preferably H, F or ethyl, very preferably H,
L^{S1} and L^{S2} identically or differently, denote H or F, preferably F.

The compounds of the general formula S are prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and are suitable for the said reactions. Use can be made here of variants which are known per se, but are not mentioned here in greater detail.

The compounds of formula S are prepared for example according to or in analogy to the procedures described in CN 106518890 A.

A general approach towards this class of compound is also given in EP 0144013 A, further examples of thienothiophene derived mesogenic compounds are described in KR20160001773 A.

If desired, the starting materials can also be formed in situ by not isolating them from the reaction mixture, but instead immediately converting them further into the compounds of the general formula S.

Preferred synthetic pathways towards compounds according to the invention are shown in the schemes below and are further illustrated by means of the working examples. The syntheses can be adapted to the particular desired compounds of the general formula S by choice of suitable starting materials.

Versatile building blocks are compounds 1, 2, 3 and 4 shown below, which can be prepared as described in Weihua Tang et al., J. Mater. Chem., 2010, 20, 1497-1505; KR20100092592 (A), JP 2012167068 A and WO 2011/119870 A1, and in which R has the meanings given above for R^{s} and may also denote alkylcyclohexyl or alkylcyclohexenyl and R′ denotes H or SiMe₃ or an equivalent protecting group known in the art.

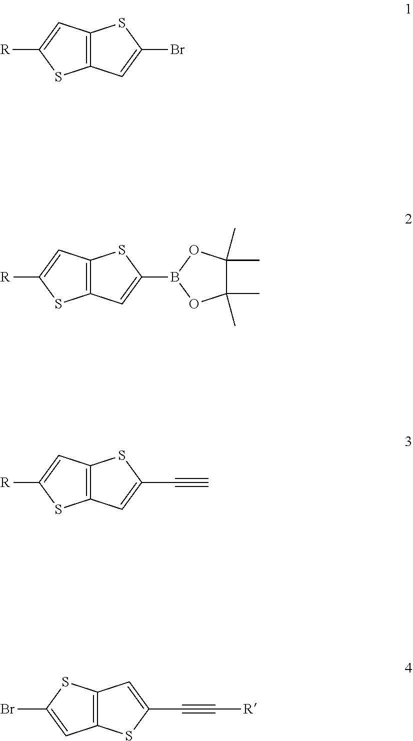

According to CN 106518890 A, the compounds of formula 1 can be used for the preparation of compounds of formula S1 by Suzuki cross coupling, and the compounds of formula 3 have been used for the preparation of compounds of formula S2 by Sonogashira coupling, with suitably substituted 4-bromo or iodoanilines. Similarly, by using a cyclic alkyl radical R, e.g. alkylcyclohexyl, the compounds of formulae S10 and S11, respectively, can be made using the same chemistry. Likewise, by using p-aminobiphenyl bromides and iodides such as e.g. compounds of the following formula

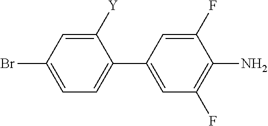

where Y is H or F, known from EP 1126006 A2, it is possible to synthesise compounds for example of formulae S4, S8 and S9. Likewise, from alkynes such as

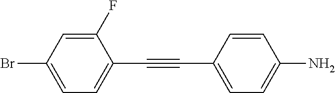 [SMILES: Nc1ccc(C#Cc2ccc(Br)cc2F)cc1]

described in Arakawa, Yuki et al., RSC Advances (2016), 6(95), 92845-92851, compounds of the type S6 are prepared.

The building block 4 shown above enables the synthesis of aryl-substituted compounds of formulae S5 and S7, exemplified by the synthesis shown in the following scheme, by for example first reacting the position carrying the bromine atom to give intermediate 5, where R′ is optionally a protecting group, followed by a Sonogashira coupling:

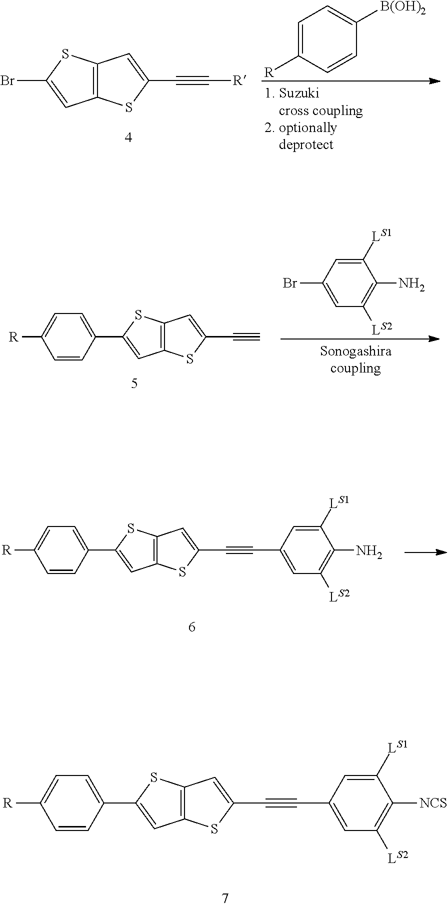

The starting materials for the synthesis of compounds with a —CF═CF— bridge are commercially available or can be synthesised according to known procedures. Preferably, these compounds are synthesised analogously to the procedures disclosed in WO 2012/069133 A1 and WO 2018/077765 A1.

The compounds of formula S according to the present invention are preferably prepared from the precursors of formula SP below,

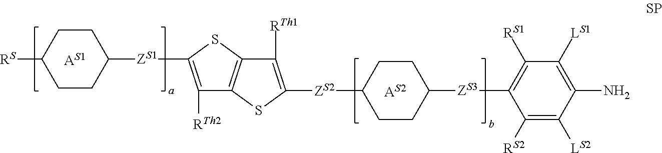

in which the groups and parameters have the meanings defined above for formula S.

The invention relates to a compound of formula SP with the proviso that if a and b are both 0, Z^{S2} denotes —CH═CH—, —CH═CF—, —CF═CH—, or —CF═CF—.

The invention further relates to a process for the preparation of a compound of formula S by reaction of a compound of formula SP with a reagent selected from the group of thiophosgene, thiocarbonyldiimidazol, carbon disulfide, carbonochloridothioic acid-O-phenyl ester, N,N,N′,N′-tetramethyl-thioperoxydicarbonic diamide, carbonothioic acid-O,O-di-2-pyridinyl ester, chloroethyl formate, 1,1′-carbonothioylbis-2(1H)-pyridinone, and the like; for a review cf. Braverman, S.; Cherkinsky, M.; Birsa, M. L., Science of Synthesis, (2005) 18, 190.

The compounds of formula IC are preferably selected from the group of the compounds of the formulae ICa to ICd, particularly preferably of formula ICb:

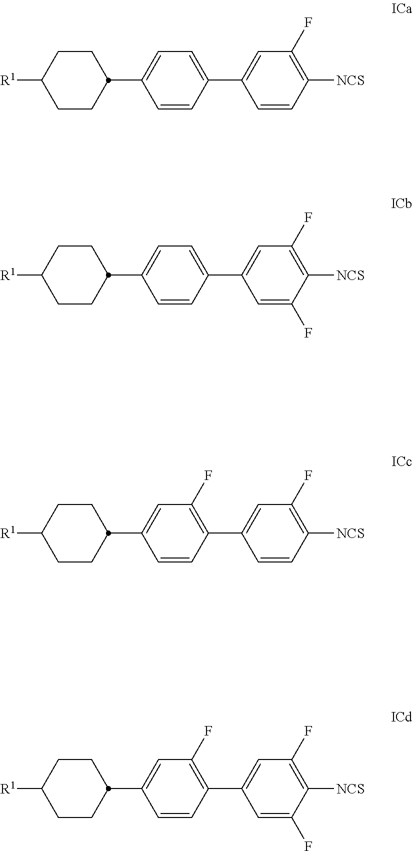

in which R¹ has the meaning indicated above for formula I and preferably denotes alkyl having 1 to 7 C atoms or alkenyl having 2 to 7 C atoms.

In a preferred embodiment of the present invention the medium comprises one or more compounds selected from the group of compounds of formulae I, II and III,

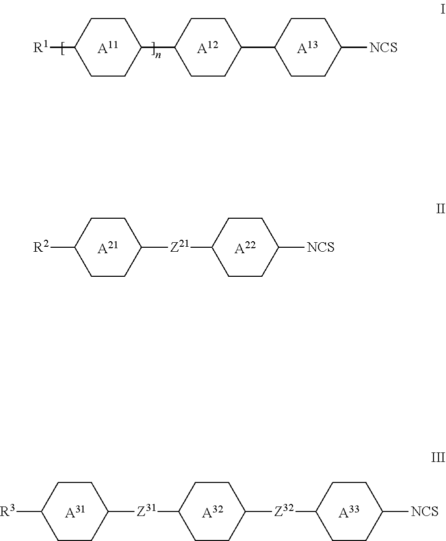

in which
R¹ denotes H, alkyl or alkoxy having 1 to 15, preferably 3 to 10 C atoms, or alkenyl, alkenyloxy or alkoxyalkyl having 2 to 15, preferably 3 to 10, C atoms, in which one or more CH₂-groups may be replaced by

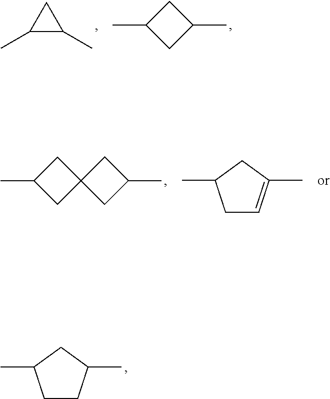

preferably alkyl or alkenyl,
n is 0, 1 or 2,

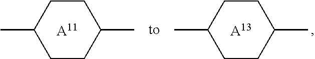

on each occurrence, independently of one another, denote

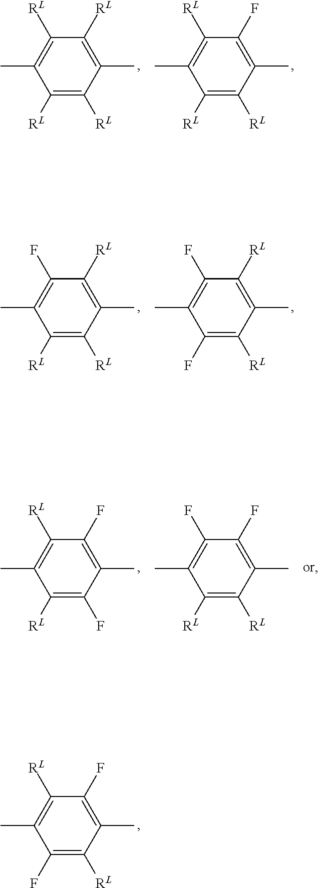

in which R^{L}, on each occurrence, identically or differently, denotes H or alkyl having 1 to 6 C atoms, preferably H, methyl or ethyl, particularly preferably H,
and wherein

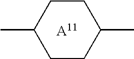

alternatively denotes

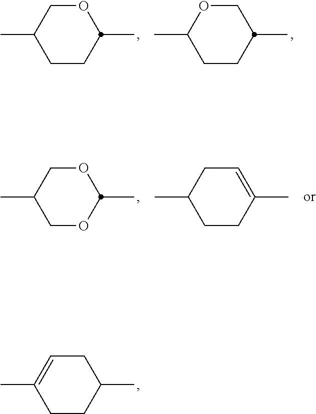

and in case n=2

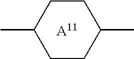

alternatively denotes

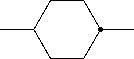

and preferably one denotes

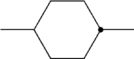

and
the other denotes

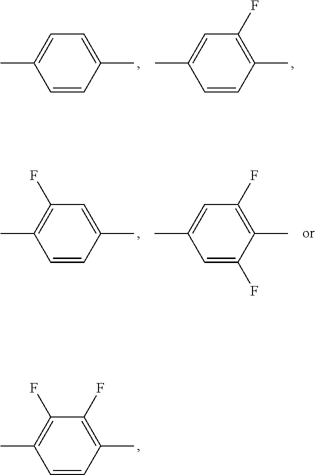

preferably

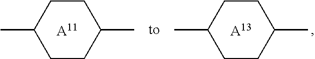

independently of one another, denote

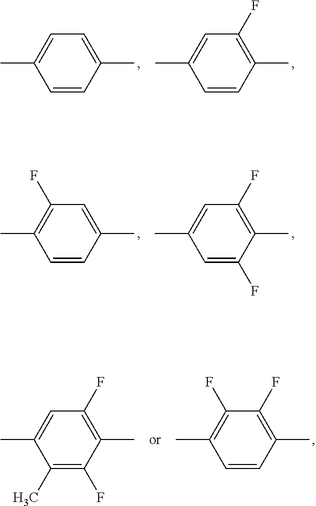

more preferably

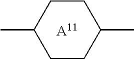

denotes

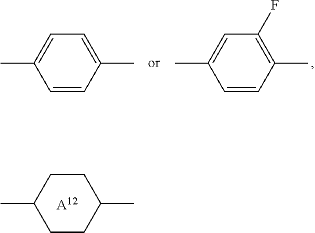

denotes

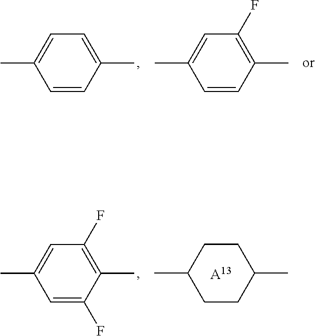

denotes

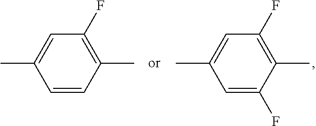

R² denotes H, alkyl or alkoxy having 1 to 15, preferably 3 to 10 C atoms, or alkenyl, alkenyloxy or alkoxyalkyl having 2 to 15, preferably 3 to 10, C atoms, in which one or more CH₂-groups may be replaced by

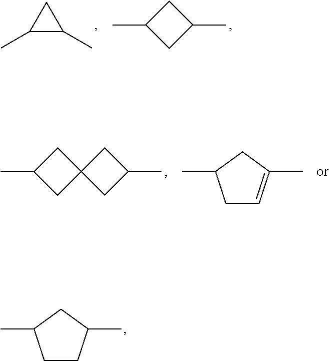

preferably alkyl or alkenyl,
Z²¹ denotes trans-CH═CH—, trans-CF═CF— or —C≡C—, preferably —C≡C— or trans-CH═CH—, and

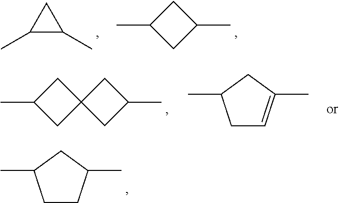

independently of one another, denote

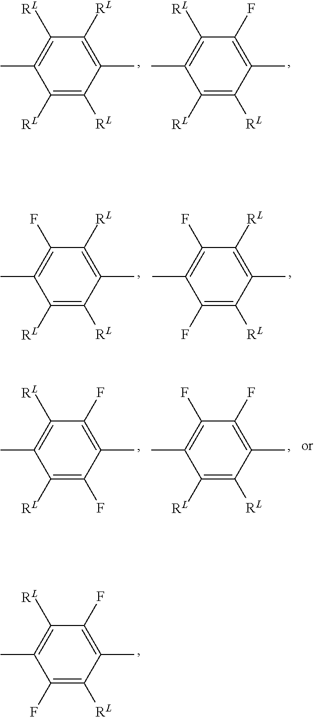

in which R^{L}, on each occurrence, identically or differently, denotes H or alkyl having 1 to 6 C atoms, preferably H, methyl or ethyl, particularly preferably H,
and wherein

preferably

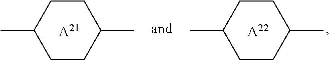

independently of one another, denote

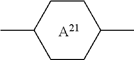

preferably denotes

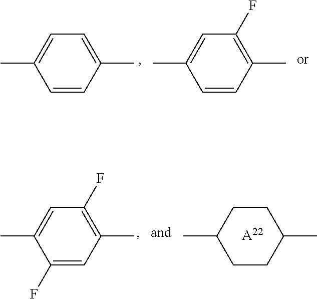

preferably denotes

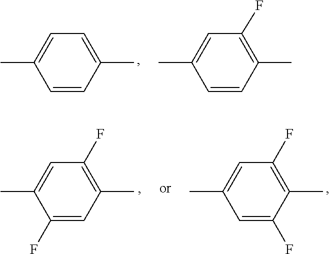

more preferably

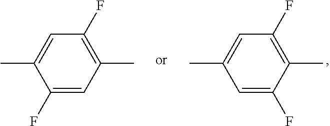

R³ denotes H, alkyl or alkoxy having 1 to 15, preferably 3 to 10 C atoms, or alkenyl, alkenyloxy or alkoxyalkyl having 2 to 15, preferably 3 to 10, C atoms, in which one or more CH₂-groups may be replaced by

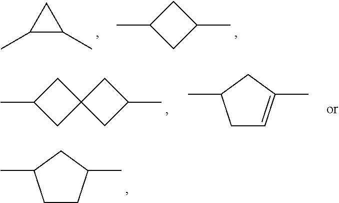

preferably alkyl or alkenyl,
one of Z³¹ and Z³², preferably Z³², denotes trans-CH═CH—, trans-CF═CF— or —C≡C— and the other one, independently thereof, denotes —C≡C—, trans-CH═CH—, trans-CF═CF— or a single bond, preferably one of them, preferably Z³² denotes —C≡C— or trans-CH═CH— and the other denotes a single bond, and

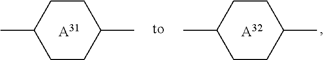

independently of one another, denote

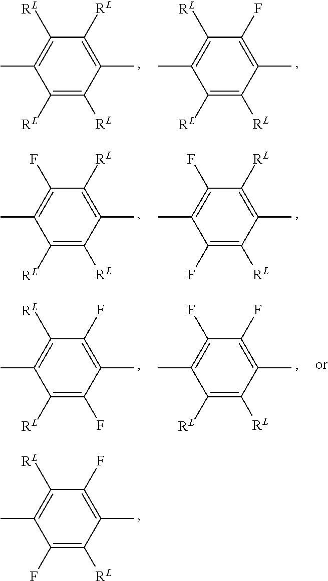

in which R^{L}, on each occurrence, identically or differently, denotes H or alkyl having 1 to 6 C atoms, preferably H, methyl or ethyl, particularly preferably H,
and wherein

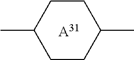

alternatively denotes

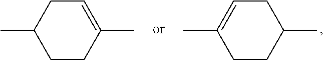

preferably

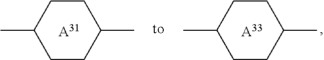

independently of one another, denote

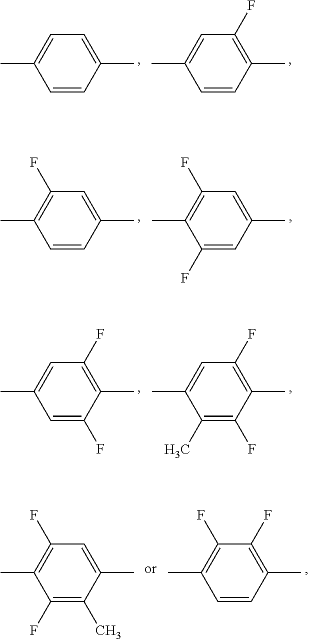

more preferably

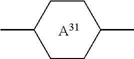

denotes

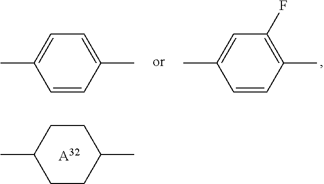

denotes

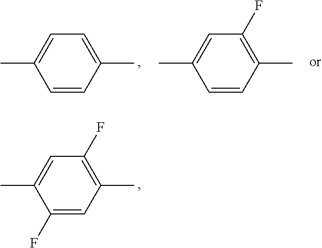

and
more preferably

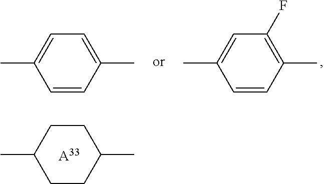

denotes

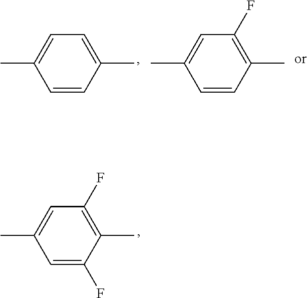

more preferably

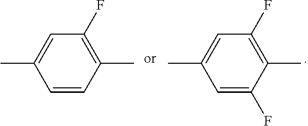

In the compounds of formulae I, II and III R^{L} preferably denotes H.

In another preferred embodiment, in the compounds of formulae I, II and III, one or two groups R^{L}, preferably one group R^{L} is different from H.

In a preferred embodiment of the present invention, the compounds of formula I are selected from the group of compounds of the formulae I-1 to I-4:

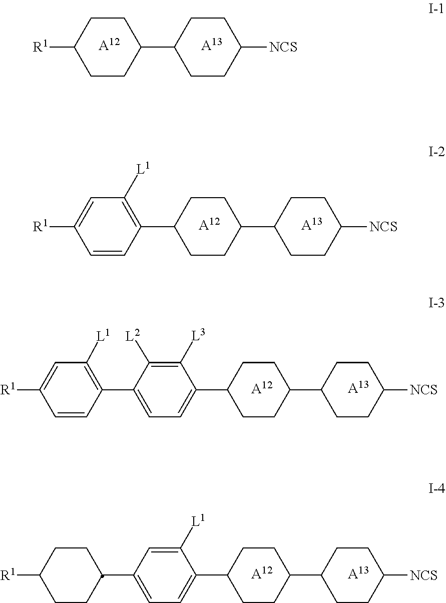

in which

L¹, L² and L³ on each occurrence, identically or differently, denote H or F, and the other groups have the respective meanings indicated above for formula I and preferably
R¹ denotes alkyl having 1 to 7 C atoms or alkenyl having 2 to 7 C atoms.

The media preferably comprise one or more compounds of formula I-1, which are preferably selected from the group of the compounds of the formulae I-1a to I-1f, preferably of formula I-1b or I-1f:

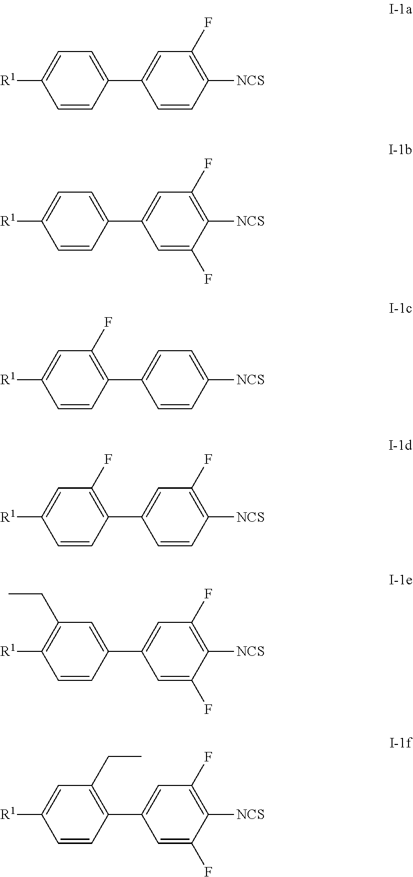

in which R¹ has the meaning indicated above for formula I and preferably denotes alkyl having 1 to 7 C atoms or alkenyl having 2 to 7 C atoms.

The media preferably comprise one or more compounds of formula I-2, which are preferably selected from the group of the compounds of the formulae I-2a to I-2e, preferably of formula I-2c:

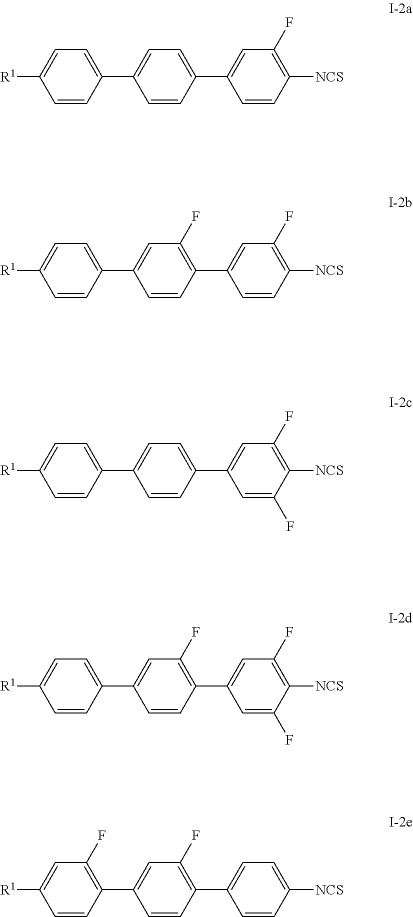

in which R¹ has the meaning indicated above for formula I and preferably denotes alkyl having 1 to 7 C atoms or alkenyl having 2 to 7 C atoms.

The media preferably comprise one or more compounds of formula I-3, which are preferably selected from the group of the compounds of the formulae I-3a to I-3d, particularly preferably of formula I-3b:

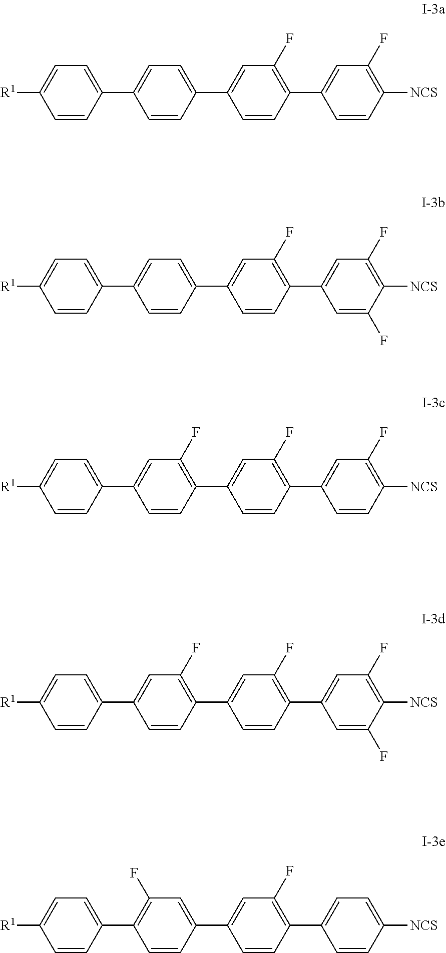

in which R¹ has the meaning indicated above for formula I and preferably denotes alkyl having 1 to 7 C atoms or alkenyl having 2 to 7 C atoms. The media preferably comprise one or more compounds of formula I-4, which are preferably selected from the group of the compounds of the formulae I-4a to I-4d, particularly preferably of formula I-4b:

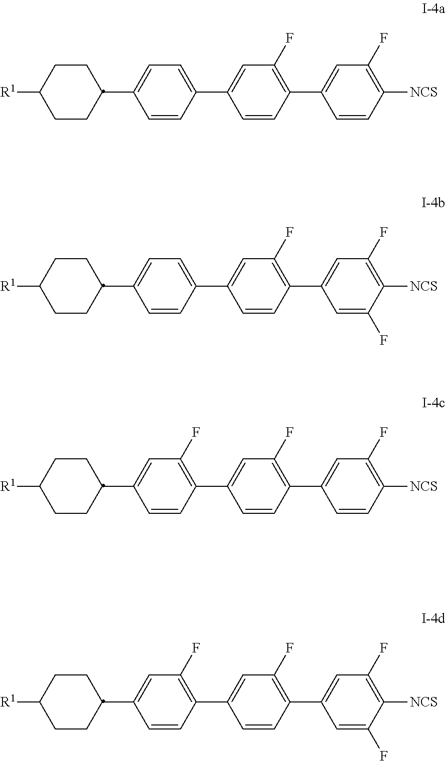

in which R¹ has the meaning indicated above for formula I and preferably denotes alkyl having 1 to 7 C atoms or alkenyl having 2 to 7 C atoms.

The media preferably comprise one or more compounds of formula II, which are preferably selected from the group of the compounds of the formulae II-1 to II-3, preferably selected from the group of the compounds of the formulae II-1 and II-2:

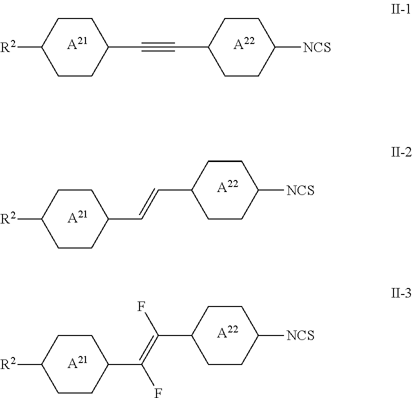

in which the parameters have the meanings given under formula II above and preferably
R² denotes H, alkyl or alkoxy having 1 to 7 C atoms or alkenyl having 2 to 7 C atoms,

and one of

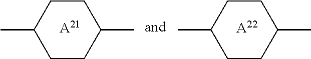

denotes

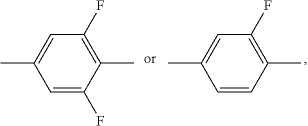

and the other, independently denotes

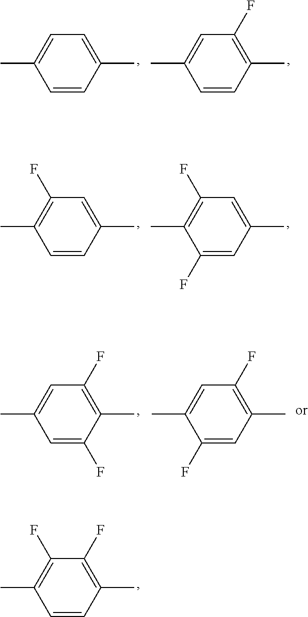

preferably

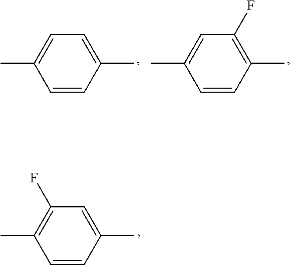

most preferably

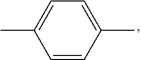

and preferably
R² denotes CₙH₂ₙ₊₁ or CH₂═CH—(CH₂)_{z}, and
n denotes an integer in the range from 0 to 15, preferably in the range from 1 to 7 and particularly preferably 1 to 5, and
z denotes 0, 1, 2, 3 or 4, preferably 0 or 2.

The compounds of formula II-1 are preferably selected from the group of the compounds of the formulae II-1a to II-1e:

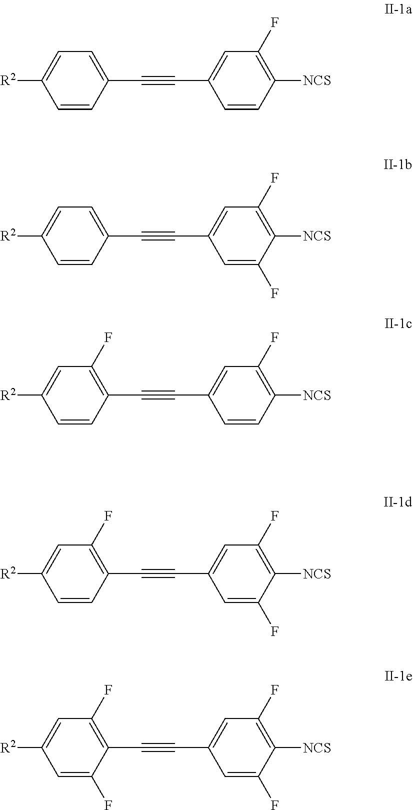

in which
R² has the meaning indicated above and preferably denotes CₙH₂ₙ₊₁ or CH₂═CH—(CH₂)z, and
n independently of one another, denote an integer in the range from 0 to 15, preferably in the range from 1 to 7 and particularly preferably 1 to 5, and
z denotes 0, 1, 2, 3 or 4, preferably 0 or 2.

The compounds of formula II-2 are preferably selected from the group of the compounds of the formulae II-2a and II-2b:

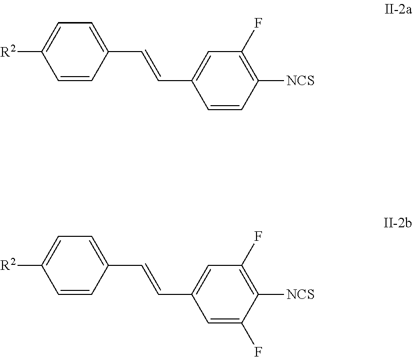

in which
R² has the meaning indicated above and preferably denotes CₙH₂ₙ₊₁ or CH₂═CH—(CH₂)z,
n denotes an integer in the range from 0 to 15, preferably in the range from 1 to 7 and particularly preferably 1 to 5, and
z denotes 0, 1, 2, 3 or 4, preferably 0 or 2.

The compounds of formula II-3 are preferably selected from the group of the compounds of the of formulae II-3a to II-3d:

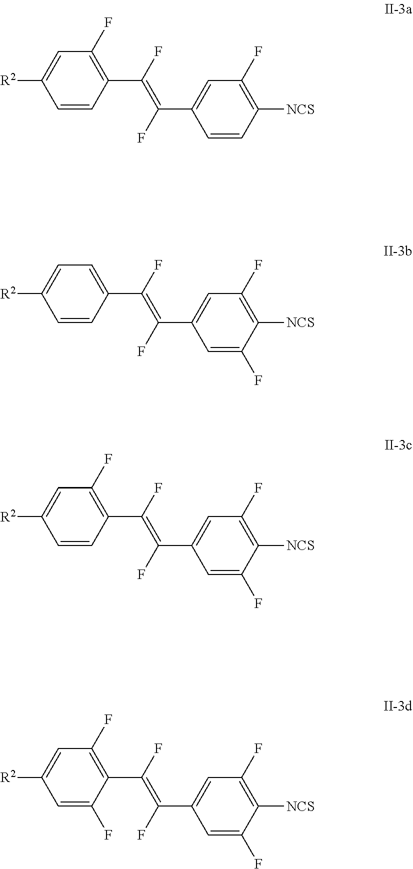

in which
R² has the meaning indicated above and preferably denotes CₙH₂ₙ₊₁ or CH₂═CH—(CH₂)z,
n denotes an integer in the range from 0 to 15, preferably in the range from 1 to 7 and particularly preferably 1 to 5, and
z denotes 0, 1, 2, 3 or 4, preferably 0 or 2.

The compounds of formula III are preferably selected from the group of the compounds of the formulae III-1 to III-6, more preferably of the formulae selected from the group of the compounds of the formulae III-1, III-2, III-3 and III-4, and particularly preferably of formula III-1:

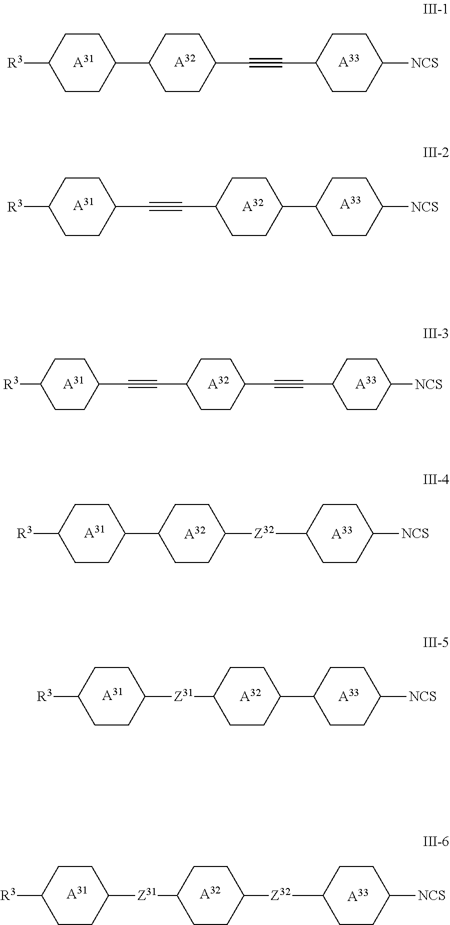

in which
Z³¹ and Z³² independently of one another denote trans-CH═CH— or trans-CF═CF—, preferably trans-CH═CH—, and in formula III-6 alternatively one of Z³¹ and Z³² may denote —C≡C— and the other parameters have the meaning given above under formula III,

and preferably
R³ denotes H, alkyl or alkoxy having 1 to 7 C atoms or alkenyl having 2 to 7 C atoms,

and one of

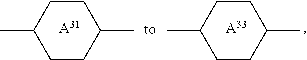

preferably

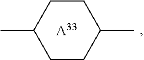

denotes

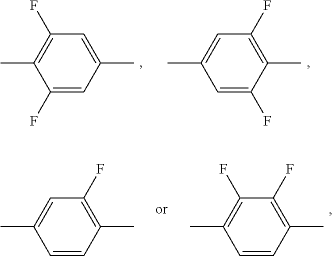

preferably

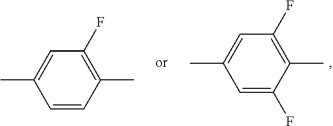

and the others, independently of one another, denote

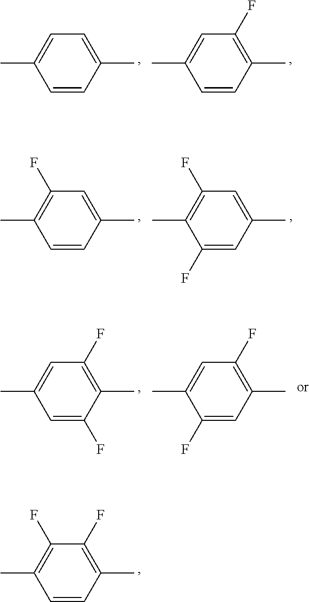

preferably

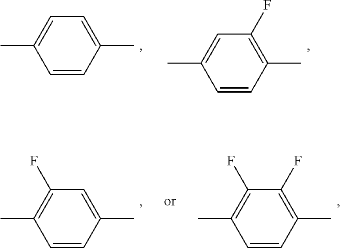

more preferably

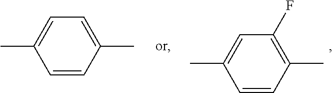

and preferably
R³ denotes CₙH₂ₙ₊₁ or CH₂═CH—(CH₂)z,
n denotes an integer in the range from 0 to 15, preferably in the range from 1 to 7 and particularly preferably 1 to 5, and
z denotes 0, 1, 2, 3 or 4, preferably 0 or 2.

The compounds of formula III-1 are preferably selected from the group of the compounds of the formulae III-1a to III-1e, more preferably selected from the group of the compounds of the formulae III-1a and III-1b, particularly preferably of formula III-1b:

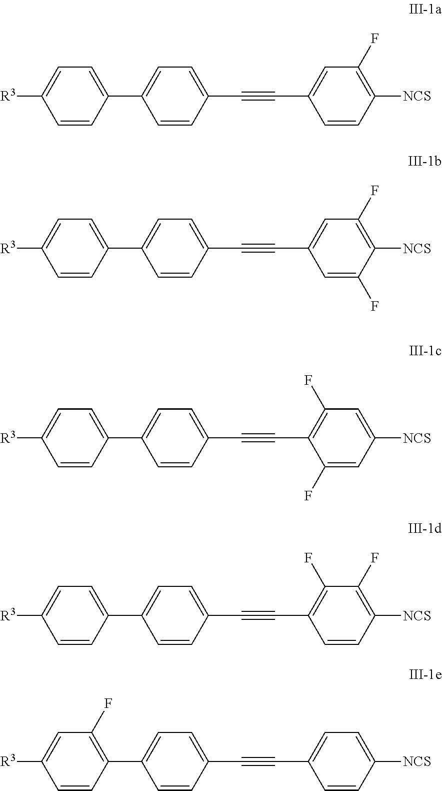

in which
R³ has the meaning indicated above and preferably denotes CₙH₂ₙ₊₁ or CH₂═CH—(CH₂)z,
n denotes an integer in the range from 0 to 15, preferably in the range from 1 to 7 and particularly preferably 1 to
z denotes 0, 1, 2, 3 or 4, preferably 0 or 2.

The compounds of formula III-2 are preferably compounds of formula III-2a to III-2h, very preferably III-2b and/or III-2h:

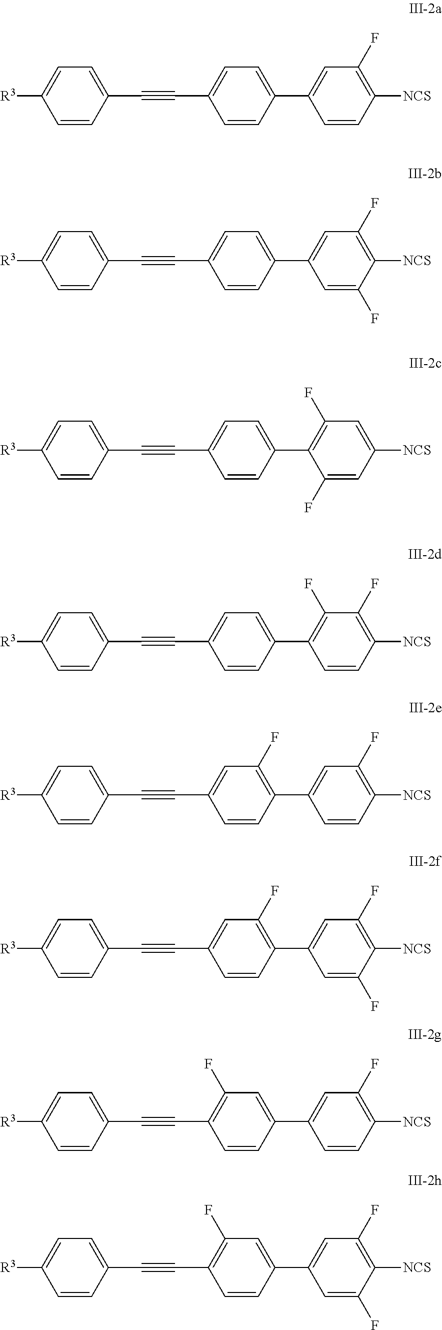

in which
R³ has the meaning indicated above and preferably denotes CₙH₂ₙ₊₁ or CH₂═CH—(CH₂)z,
n denotes an integer in the range from 0 to 15, preferably in the range from 1 to 7 and particularly preferably 1 to 5, and
z denotes 0, 1, 2, 3 or 4, preferably 0 or 2.

The compounds of formula III-5 are preferably selected from the compounds of formula III-5a:

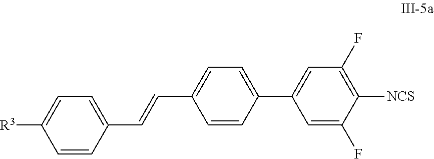

R³ has the meaning indicated above for formula III-5 and preferably denotes CₙH₂ₙ₊₁, in which
n denotes an integer in the range from 0 to 7, preferably in the range from 1 to 5.

In a preferred embodiment, the media according to the invention comprise one or more compounds selected from the group of compounds of the formulae IIA-1-1 to IIA-1-12, very preferably IIA-1-1 or IIA-1-2:

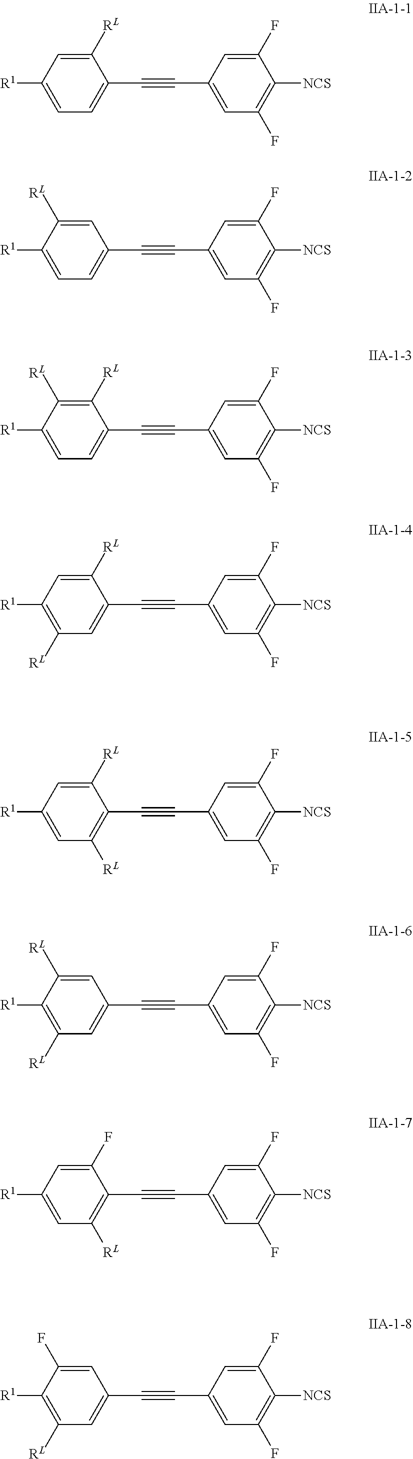

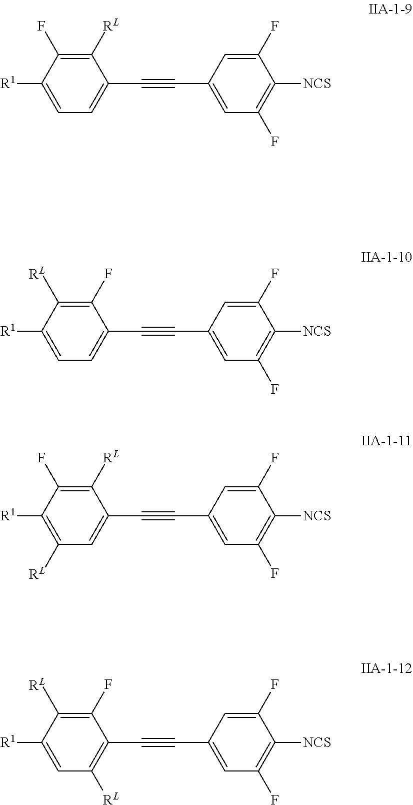

in which
R¹ denotes alkyl or alkenyl having up to 7 C atoms, preferably ethyl, n-propyl, n-butyl or n-pentyl, n-hexyl,
R^{L} on each occurrence, the same or differently, denotes alkyl or alkenyl having 1 to 5 C atoms, or cycloalkyl or cycloalkenyl each having 3 to 6 C atoms,
   preferably methyl, ethyl, n-propyl, n-butyl, isopropyl, cyclopropyl, cyclobutyl, cyclopentyl or cyclopent-1-enyl, very preferably ethyl.

In a preferred embodiment, the medium according to the present invention comprises one or more compounds of formula IIIC

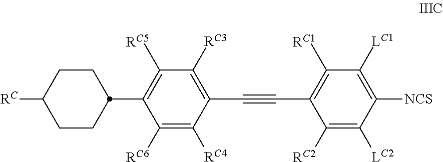

in which
R^{C} denotes H, alkyl or alkoxy having 1 to 12 C atoms, or alkenyl, alkenyloxy or alkoxyalkyl having 2 to 12 C atoms, in which one or more CH₂-groups may be replaced by

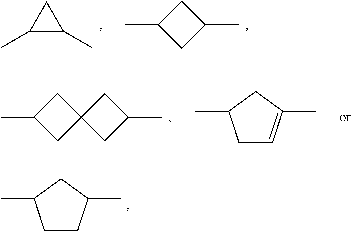

L^{C1}, L^{C2} identically or differently, denote H, Cl or F,
R^{C1}, R^{C2} identically or differently, denote H or alkyl having 1 to 6 C atoms, or cyclopropyl, cyclobutyl or cyclopentyl,
R^{C3}, R^{C4},
R^{C5} and R^{C6} identically or differently, denote H, Cl, F or alkyl having 1 to 6 C atoms, or cyclopropyl, cyclobutyl or cyclopentyl.

Preferably, the compounds of formula C are selected from the group of compounds of the formulae IIIC-1 to IIIC-12

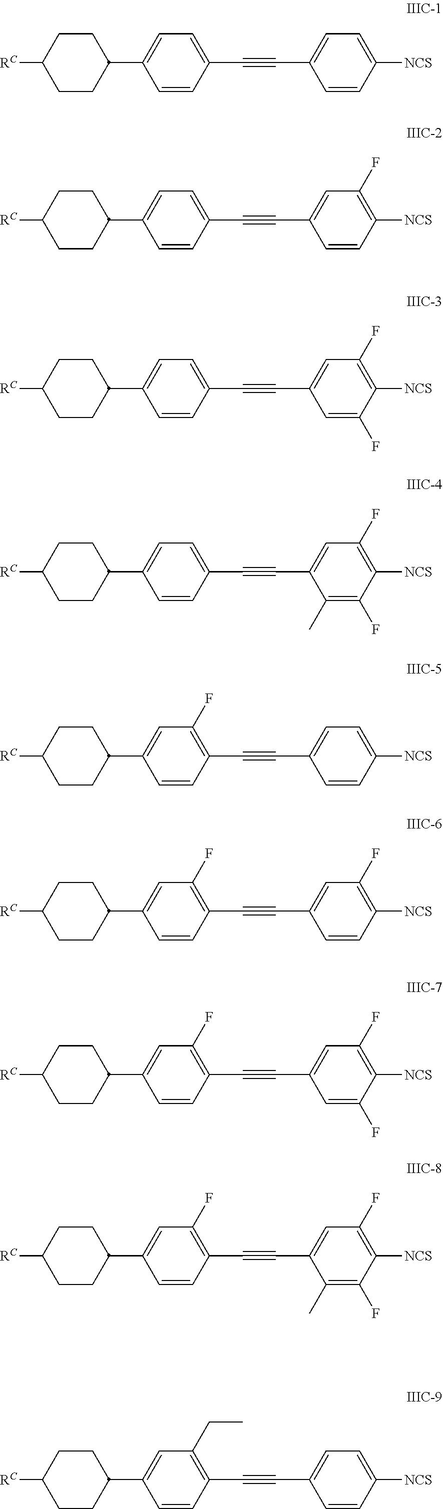

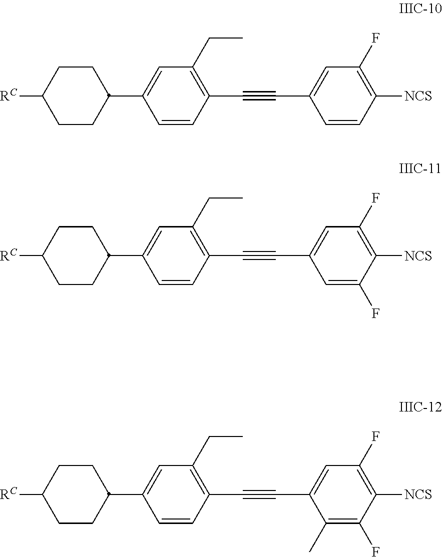

in which
R^{C} has one of the meanings indicated above for formula C and preferably denotes alkyl or alkenyl having 2 to 6 C atoms, in which one or more CH₂-groups may be replaced by

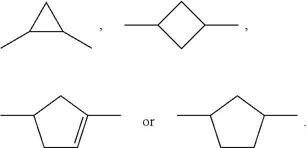

In a preferred embodiment, the medium according to the invention comprises one or more compounds of formula T

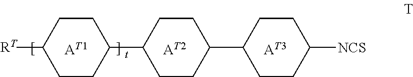

in which
R^{T} denotes halogen, CN, NCS, R^{F}, R^{F}—O— or R^{F}—S—, wherein R^{F} denotes fluorinated alkyl or fluorinated alkenyl having up to 12 C atoms,

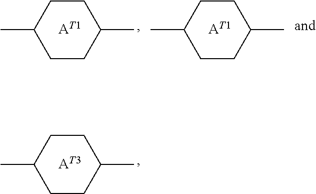

on each occurrence, independently of one another, denote

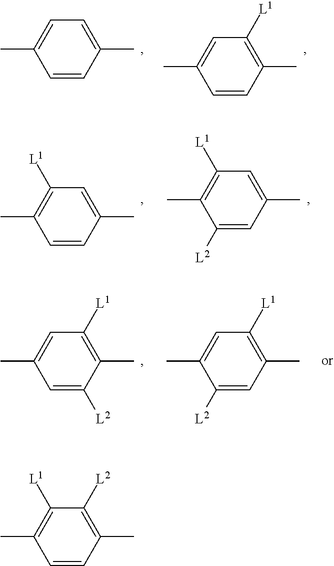

preferably

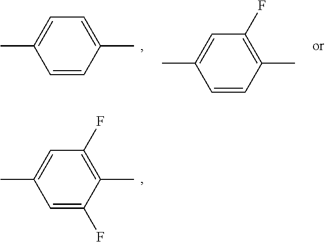

L¹ and L² identically or differently, denote Cl, F, alkyl having 1 to 6 C atoms or cyclopropyl, cyclobutyl or cyclopentyl, preferably F, and
t is 0, 1 or 2, preferably 1.

Preferably, the one or more compounds of formula T are selected from the group of compounds of the formulae T-1 and T-2,

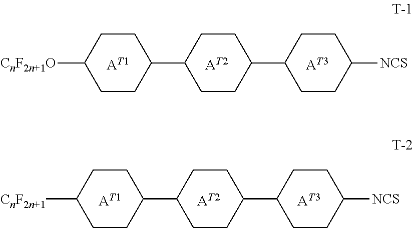

in which

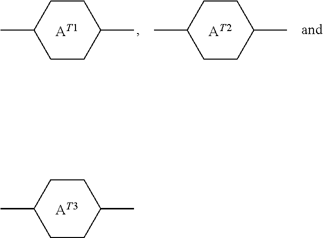

have the meanings given above for formula T, and
n is 1, 2, 3, 4, 5, 6 or 7, preferably 1, 2, 3 or 4, particularly preferably 1.

In a particularly preferred embodiment of the present invention the media comprise one or more compounds of formula T-1.

Preferred compounds of formula T-1 are selected from the group of compounds of the following sub-formulae:

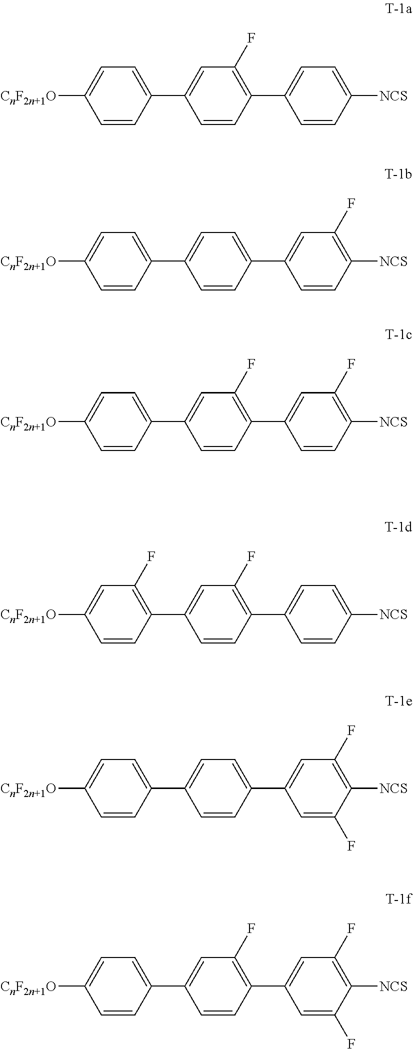

in which n is 1, 2, 3 or 4, preferably 1.

In another particularly preferred embodiment of the present invention the media comprise one or more compounds of formula T-2.

Preferred compounds of formula T-2 are selected from the group of compounds of the following sub-formulae:

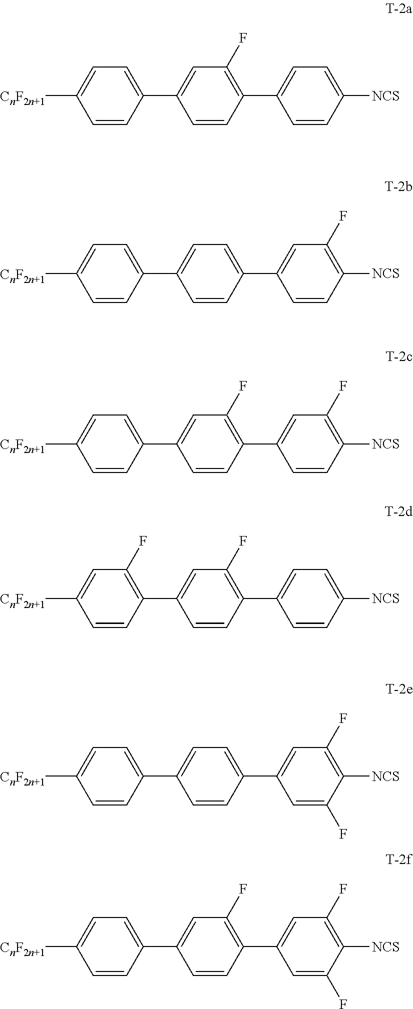

in which n is 1, 2, 3 or 4, preferably 1.

Additionally, the liquid-crystalline media according to the present invention in a certain embodiment, which may be the same or different from the previous preferred embodiments preferably comprise one or more compounds of formula IV,

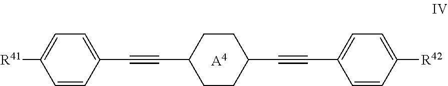

in which

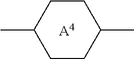

denotes

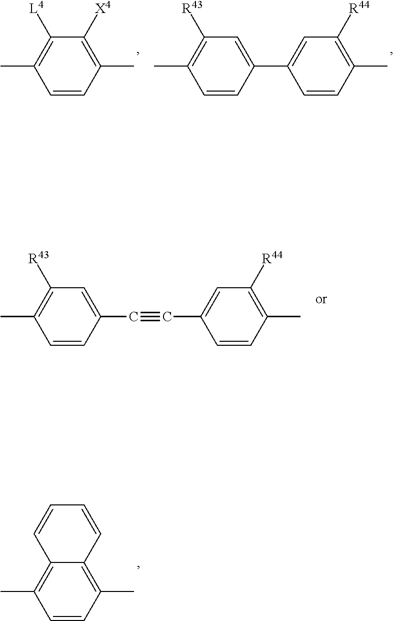

preferably

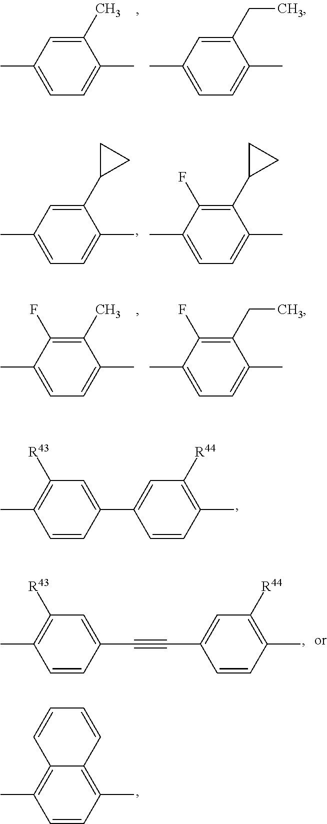

particularly preferably

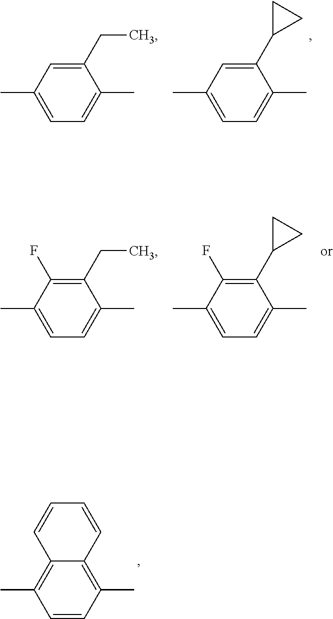

L⁴ denotes alkyl having 1 to 6 C atoms, cycloalkyl having 3 to 6 C atoms or cycloalkenyl having 4 to 6 C atoms, preferably CH₃, C₂H₅, n-C₃H₇ (—(CH₂)₂CH₃), i-C₃H₇ (—CH(CH₃)₂), cyclopropyl, cyclobutyl, cyclohexyl, cyclopent-1-enyl or cyclohex-1-enyl, and particularly preferably CH₃, C₂H₅, cyclopropyl or cyclobutyl,
X⁴ denotes H, alkyl having 1 to 3 C atoms or halogen, preferably H, F or Cl, and particularly preferably H or F and very particularly preferably F,
R⁴¹ to R⁴⁴, independently of one another, denote alkyl or alkoxy, each having 1 to 15 C atoms, alkenyl, alkenyloxy or alkoxyalkyl, each having 2 to 15 C atoms, or cycloalkyl, alkylcycloalkyl, cycloalkenyl, alkylcycloalkenyl, alkylcycloalkylalkyl or alkylcycloalkenylalkyl, each having up to 15 C atoms, and alternatively one of R⁴³ and R⁴⁴ or both also denote H,

preferably
R⁴¹ and R⁴², independently of one another, denote alkyl or alkoxy, each having 1 to 7 C atoms, or alkenyl, alkenyloxy or alkoxyalkyl, each having 2 to 7 C atoms,

particularly preferably
R⁴¹ denotes alkyl having 1 to 7 C atoms or alkenyl, alkenyloxy or alkoxyalkyl, each having 2 to 7 C atoms, and

particularly preferably
R⁴² denotes alkyl or alkoxy, each having 1 to 7 C atoms, and

preferably
R⁴³ and R⁴⁴ denote H, alkyl having 1 to 5 C atoms, cycloalkyl or cycloalkenyl having 3 to 7 C atoms, alkylcyclohexyl or cyclohexylalkyl, each having 4 to 12 C atoms, or alkylcyclohexylalkyl having 5 to 15 C atoms, particularly preferably cyclopropyl, cyclobutyl or cyclohexyl, and very particularly preferably at least one of R⁴³ and R⁴⁴ denotes n-alkyl, particularly preferably methyl, ethyl or n-propyl, and the other denotes H or n-alkyl, particularly preferably H, methyl, ethyl or n-propyl.

In a preferred embodiment of the present application, the liquid-crystal medium additionally comprises one or more compounds selected from the group of compounds of the formulae V, VI, VII, VIII and IX:

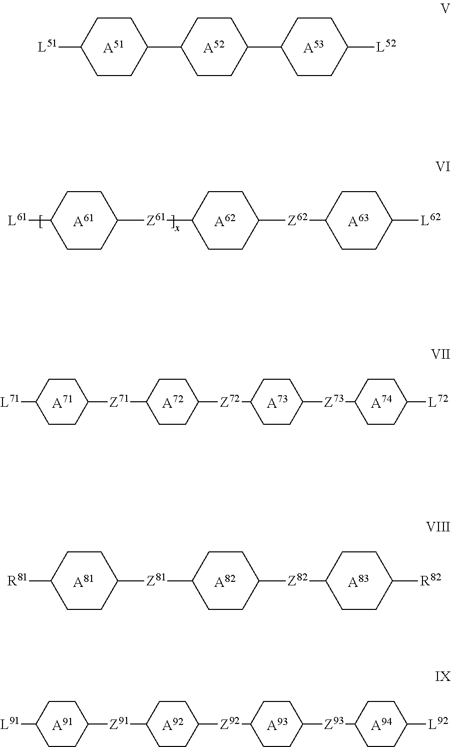

in which
L⁵¹ denotes R⁵¹ or X⁵¹,
L⁵² denotes R⁵² or X⁵²,
R⁵¹ and R⁵², independently of one another, denote H, alkyl or alkoxy having 1 to 15, preferably 3 to 10, C atoms or alkenyl, alkenyloxy or alkoxyalkyl having 2 to 15, preferably 3 to 10, C atoms, preferably alkyl or alkenyl,
X⁵¹ and X⁵², independently of one another, denote H, F, Cl, —CN, SF₅, fluorinated alkyl or fluorinated alkoxy having 1 to 7 C atoms or fluorinated alkenyl, fluorinated alkenyloxy or fluorinated alkoxyalkyl having 2 to 7 C atoms, preferably fluorinated alkoxy, fluorinated alkenyloxy, F or Cl, and

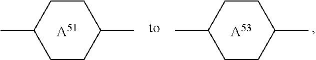

independently of one another, denote

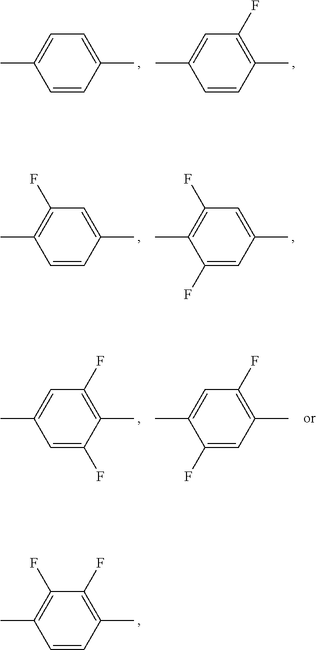

preferably

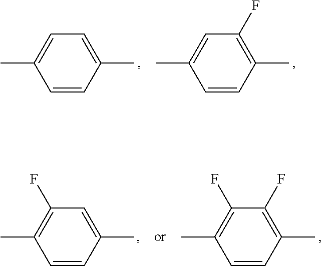

L⁶¹ denotes R⁶¹ and, in the case where Z⁶¹ and/or Z⁶² denote trans-CH═CH— or trans-CF═CF—, alternatively also denotes X⁶¹,
L⁶² denotes R⁶² and, in the case where Z⁶¹ and/or Z⁶² denote trans-CH═CH— or trans-CF═CF—, alternatively also denotes X⁶²,
R⁶¹ and R⁶², independently of one another, denote H, alkyl or alkoxy having 1 to 15, preferably 3 to 10, C atoms or alkenyl, alkenyloxy or alkoxyalkyl having 2 to 15, preferably 3 to 10, C atoms, preferably alkyl or alkenyl,
X⁶¹ and X⁶², independently of one another, denote F or Cl, —CN, SF₅, fluorinated alkyl or alkoxy having 1 to 7 C atoms or fluorinated alkenyl, alkenyloxy or alkoxyalkyl having 2 to 7 C atoms,

one of
Z⁶¹ and Z⁶² denotes trans-CH═CH—, trans-CF═CF— or —C≡C— and the other, independently thereof, denotes trans-CH═CH—, trans-CF═CF— or a single bond, preferably one of them denotes —C≡C— or trans-CH═CH— and the other denotes a single bond, and

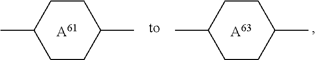

independently of one another, denote

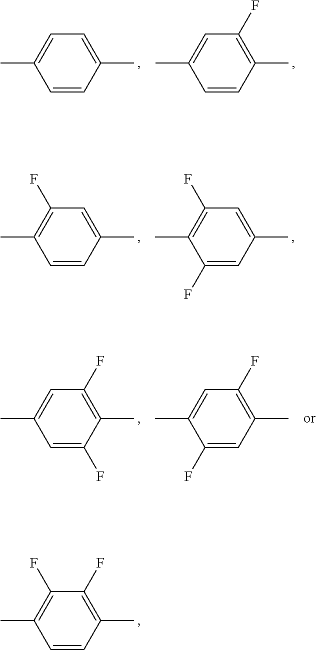

preferably

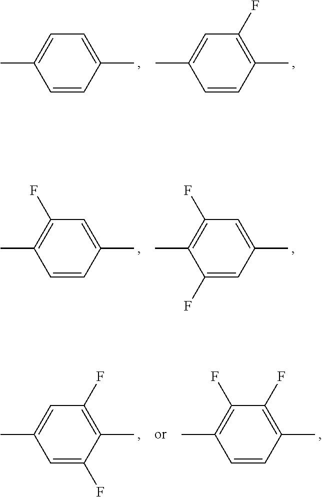

and
x denotes 0 or 1,
L⁷¹ denotes R⁷¹ or X⁷¹,
L⁷² denotes R⁷² or X⁷²,
R⁷¹ and R⁷², independently of one another, denote H, alkyl or alkoxy having 1 to 15, preferably 3 to 10, C atoms or alkenyl, alkenyloxy or alkoxyalkyl having 2 to 15, preferably 3 to 10, C atoms, preferably alkyl or alkenyl,
X⁷¹ and X⁷², independently of one another, denote H, F, Cl, —CN, —NCS, —SF₅, fluorinated alkyl or fluorinated alkoxy having 1 to 7 C atoms or fluorinated alkenyl, or fluorinated alkenyloxy or fluorinated alkoxyalkyl having 2 to 7 C atoms, preferably fluorinated alkoxy, fluorinated alkenyloxy, F or Cl, and
Z⁷¹ to Z⁷³, independently of one another, denote trans-CH═CH—, trans-CF═CF—, —C≡C— or a single bond, preferably one or more of them denote a single bond, particularly preferably all denote a single bond and

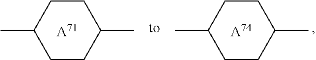

independently of one another, denote

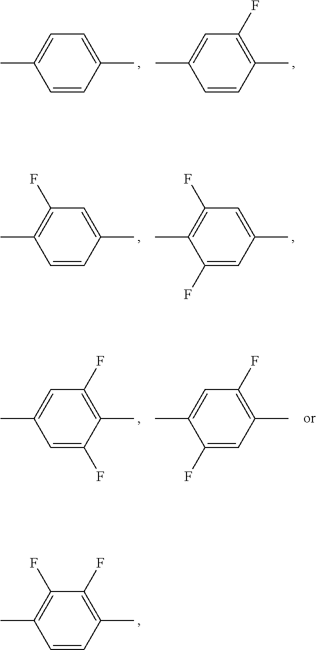

preferably

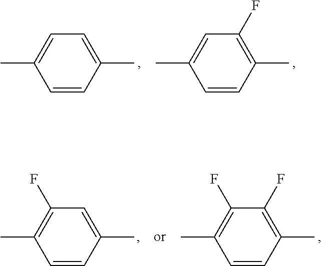

R⁸¹ and R⁸², independently of one another, denote H, alkyl or alkoxy having 1 to 15, preferably 3 to 10, C atoms or alkenyl, alkenyloxy or alkoxyalkyl having 2 to 15, preferably 3 to 10, C atoms, preferably alkyl or alkenyl,

one of
Z⁸¹ and Z⁸² denotes trans-CH═CH—, trans-CF═CF— or —C≡C— and the other, independently thereof, denotes trans-CH═CH—, trans-CF═CF— or a single bond, preferably one of them denotes —C≡C— or trans-CH═CH— and the other denotes a single bond, and

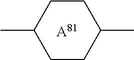

denotes

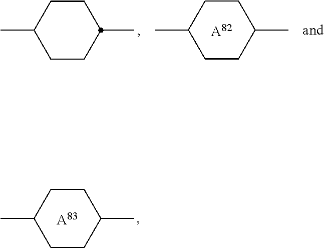

independently of one another, denote

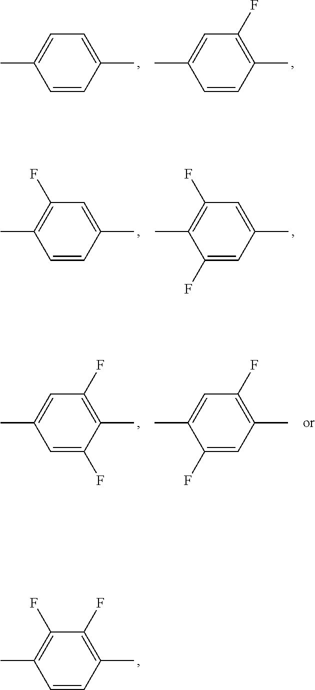

L⁹¹ denotes R⁹¹ or X⁹¹,
L⁹² denotes R⁹² or X⁹²,
R⁹¹ and R⁹², independently of one another, denote H, alkyl or alkoxy having 1 to 15, preferably 3 to 10, C atoms or alkenyl, alkenyloxy or alkoxyalkyl having 2 to 15, preferably 3 to 10, C atoms, preferably alkyl or alkenyl,
X⁹¹ and X⁹², independently of one another, denote H, F, Cl, —CN, —NCS, —SF₅, fluorinated alkyl or fluorinated alkoxy having 1 to 7 C atoms or fluorinated alkenyl, or fluorinated alkenyloxy or fluorinated alkoxyalkyl having 2 to 7 C atoms, preferably fluorinated alkoxy, fluorinated alkenyloxy, F or Cl, and
Z⁹¹ to Z⁹³, independently of one another, denote trans-CH═CH—, trans-CF═CF—, —C≡C— or a single bond, preferably one or more of them denotes a single bond, and particularly preferably all denote a single bond,

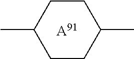

denotes

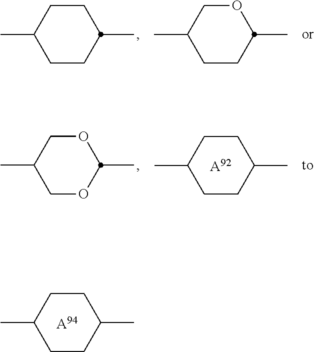

independently of one another, denote

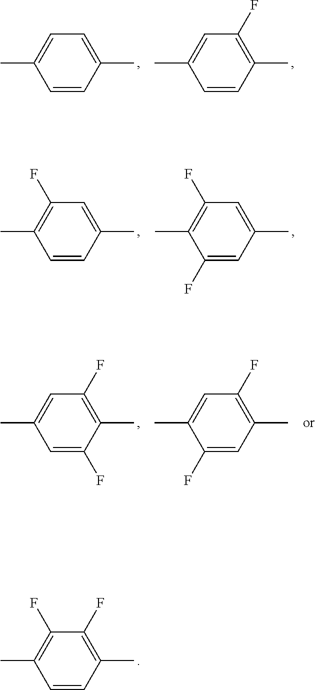

In a preferred embodiment of the present invention, the liquid-crystal medium comprises one or more compounds of the formula V, preferably selected from the group of the compounds of the formulae V-1 to V-3, preferably of the formulae V-1 and/or V-2 and/or V-3, preferably of the formulae V-1 and V-2:

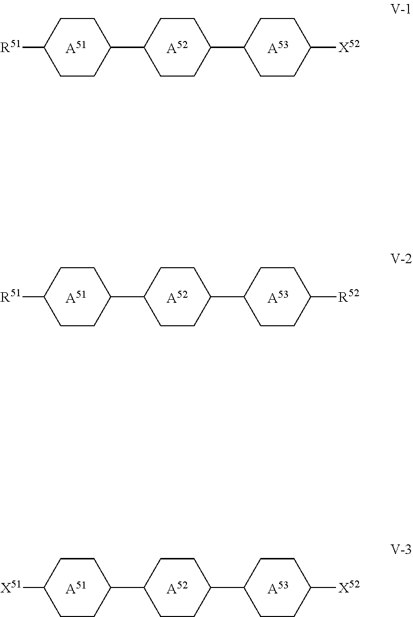

in which the parameters have the respective meanings indicated above for formula V and preferably
R⁵¹ denotes alkyl having 1 to 7 C atoms or alkenyl having 2 to 7 C atoms,
R⁵² denotes alkyl having 1 to 7 C atoms or alkenyl having 2 to 7 C atoms or alkoxy having 1 to 7 C atoms,
X⁵¹ and X⁵², independently of one another, denote F, Cl, —OCF₃, —CF₃, —CN or —SF₅, preferably F, Cl, —OCF₃ or —CN.

The compounds of the formula V-1 are preferably selected from the group of the compounds of the formulae V-1a to V-1d, preferably V-1c and V-1d:

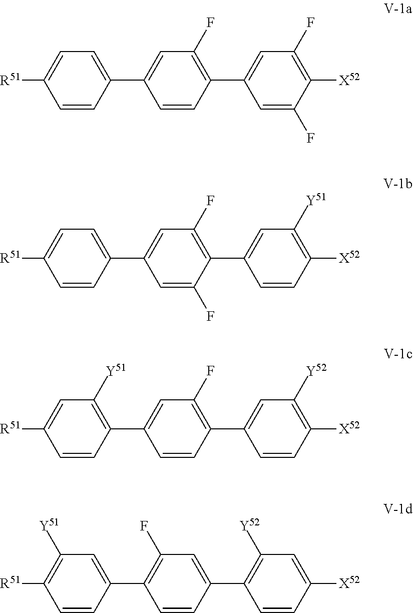

in which the parameters have the respective meanings indicated above for formula V-1 and in which
Y⁵¹ and Y⁵², in each case independently of one another, denote H or F, and preferably
R⁵¹ denotes alkyl or alkenyl, and
X⁵¹ denotes F, Cl or —OCF₃.

The compounds of the formula V-2 are preferably selected from the group of the compounds of the formulae V-2a to V-2e and/or from the group of the compounds of the formulae V-2f and V-2g:

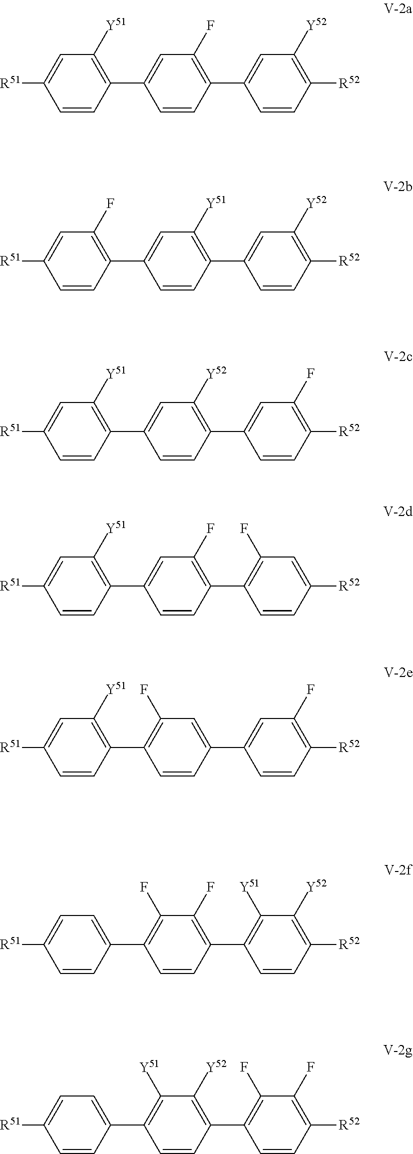

where in each case the compounds of the formula V-2a are excluded from the compounds of the formulae V-2b and V-2c, the compounds of the formula V-2b are excluded from the compounds of the formula V-2c and the compounds of the formula V-2e are excluded from the compounds of the formula V-2f, and

in which the parameters have the respective meanings indicated above for formula V-1 and in which
Y⁵¹ and Y⁵², in each case independently of one another, denote H or F, and preferably
Y⁵¹ and Y⁵² denotes H and the other denotes H or F, preferably likewise denotes H.

The compounds of the formula V-3 are preferably compounds of the formula V-3a:

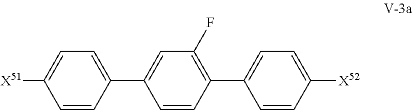

in which the parameters have the respective meanings indicated above for formula V-1 and in which preferably
X⁵¹ denotes F, Cl, preferably F,
X⁵² denotes F, Cl or —OCF₃, preferably —OCF₃.

The compounds of the formula V-1a are preferably selected from the group of the compounds of the formulae V-1a-1 and V-1a-2:

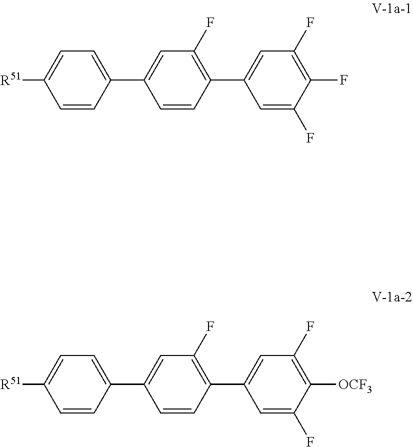

in which
R⁵¹ has the meaning indicated above and preferably denotes CₙH₂ₙ₊₁, in which
n denotes an integer in the range from 0 to 7, preferably in the range from 1 to 5 and particularly preferably 3 or 7.

The compounds of the formula V-1b are preferably compounds of the formula V-1 b-1:

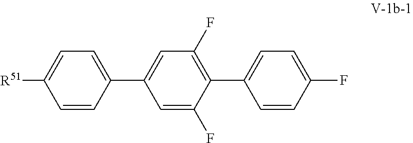

in which
R⁵¹ has the meaning indicated above and preferably denotes CₙH₂ₙ₊₁, in which
n denotes an integer in the range from 0 to 15, preferably in the range from 1 to 7 and particularly preferably 1 to 5.

The compounds of the formula V-1c are preferably selected from the group of the compounds of the formulae V-1c-1 to V-1c-4, particularly preferably selected from the group of the compounds of the formulae V-1c-1 and V-1c-2:

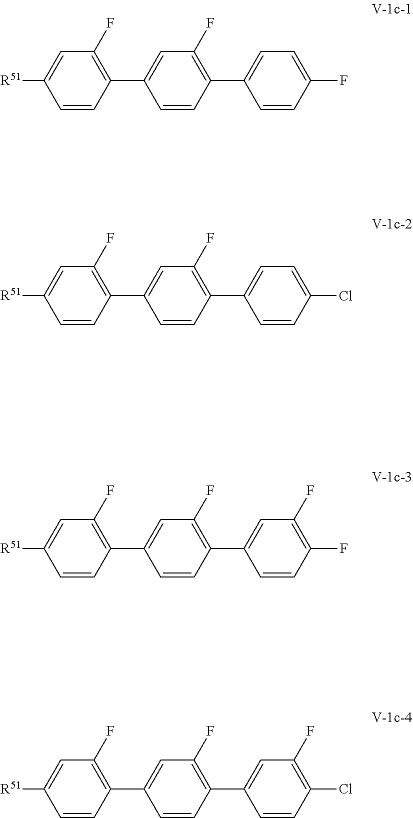

in which
R⁵¹ has the meaning indicated above and preferably denotes CₙH₂ₙ₊₁, in which
n denotes an integer in the range from 0 to 15, preferably in the range from 1 to 7 and particularly preferably 1 to 5.

The compounds of the formula V-1d are preferably selected from the group of the compounds of the formulae V-1d-1 and V-1d-2, particularly preferably the compound of the formula V-1d-2:

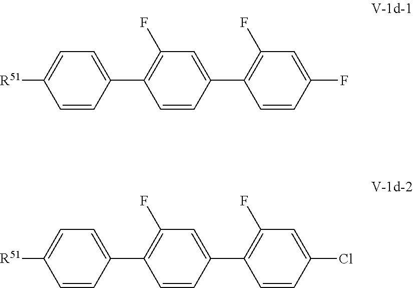

in which
R⁵¹ has the meaning indicated above and preferably denotes CₙH₂ₙ₊₁, in which
n denotes an integer in the range from 0 to 15, preferably in the range from 1 to 7 and particularly preferably 1 to 5.

The compounds of the formula V-2a are preferably selected from the group of the compounds of the formulae V-2a-1 and V-2a-2, particularly preferably the compounds of the formula V-2a-1:

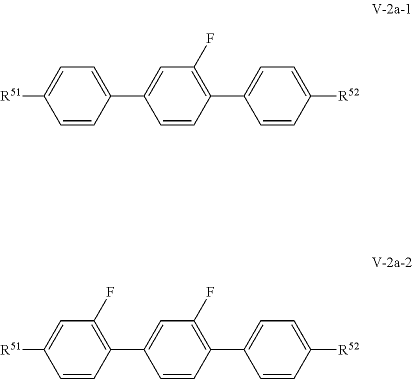

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

Preferred combinations of (R⁵¹ and R⁵²), in particular in the case of formula V-2a-1, are (CₙH₂ₙ₊₁ and CₘH₂ₘ₊₁), (CₙH₂ₙ₊₁ and O—CₘH₂ₘ₊₁), (CH₂═CH—(CH₂)z and CₘH₂ₘ₊₁), (CH₂═CH—(CH₂)z and O—CₘH₂ₘ₊₁) and (CₙH₂ₙ₊₁ and (CH₂)z-CH═CH₂).

Preferred compounds of the formula V-2b are the compounds of the formula V-2b-1:

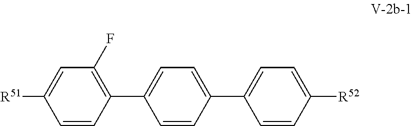

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combination of (R⁵¹ and R⁵²) here is, in particular, (CₙH₂ₙ₊₁ and CₘH₂ₘ₊₁).

Preferred compounds of the formula V-2c are the compounds of the formula V-2c-1:

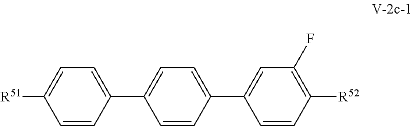

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combination of (R⁵¹ and R⁵²) here is, in particular, (CₙH₂ₙ₊₁ and CₘH₂ₘ₊₁).

Preferred compounds of the formula V-2d are the compounds of the formula V-2d-1:

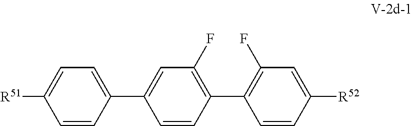

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combination of (R⁵¹ and R⁵²) here is, in particular, (CₙH₂ₙ₊₁ and CₘH₂ₘ₊₁).

Preferred compounds of the formula V-2e are the compounds of the formula V-2e-1:

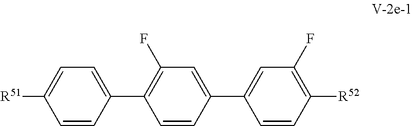

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combination of (R⁵¹ and R⁵²) here is, in particular, (CₙH₂ₙ₊₁ and O—CₘH₂ₘ₊₁).

Preferred compounds of the formula V-2f are the compounds of the formula V-2f-1:

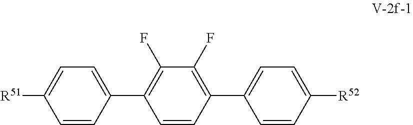

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁵¹ and R⁵²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

Preferred compounds of the formula V-2g are the compounds of the formula V-2g-1:

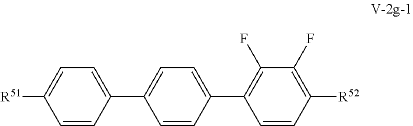

in which
R⁵¹ has the meaning indicated above and preferably denotes CₙH₂ₙ₊₁ or CH₂═CH—(CH₂)z, and
R⁵² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁵¹ and R⁵²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and O—CₘH₂ₘ₊₁).

The compounds of the formula VI are preferably selected from the group of the compounds of the formulae VI-1 to VI-5:

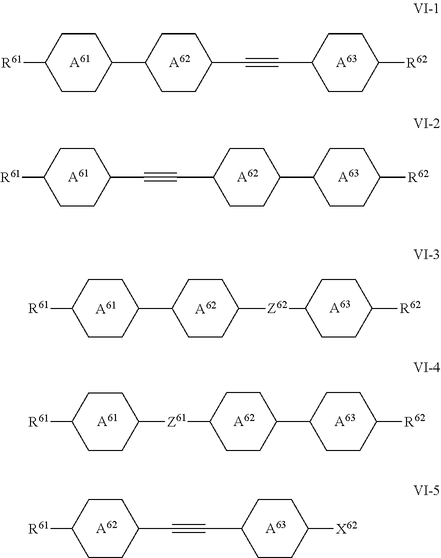

in which
Z⁶¹ and Z⁶² denote trans-CH═CH— or trans-CF═CF—, preferably trans-CH═CH—, and the other occurring groups and parameters have the meaning given above under formula VI,

and preferably
R⁶¹ and R⁶², independently of one another, denote H, alkyl or alkoxy having 1 to 7 C atoms or alkenyl having 2 to 7 C atoms,
X⁶² denotes F, Cl, —OCF₃ or —CN,

The compounds of the formula VI-1 are preferably selected from the group of the compounds of the formulae VI-1a and VI-1b, more preferably selected from compounds of the formula VI-1a:

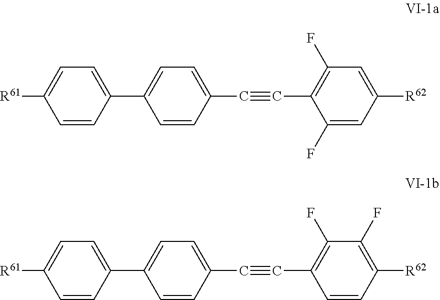

in which
R⁶¹ has the meaning indicated above and preferably denotes CₙH₂ₙ₊₁ or CH₂═CH—(CH₂)z, and
R⁶² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁶¹ and R⁶²) here are, in particular, (CₙH₂ₙ₊₁ and CₘH₂ₘ₊₁) and (CₙH₂ₙ₊₁ and O—CₘH₂ₘ₊₁), in the case of formula VI-1a particularly preferably (CₙH₂ₙ₊₁ and CₘH₂ₘ₊₁) and in the case of formula VI-1b particularly preferably (CₙH₂ₙ₊₁ and O—CₘH₂ₘ₊₁).

The compounds of the formula VI-2 are preferably selected from the compounds of the formula VI-2a to VI-2c:

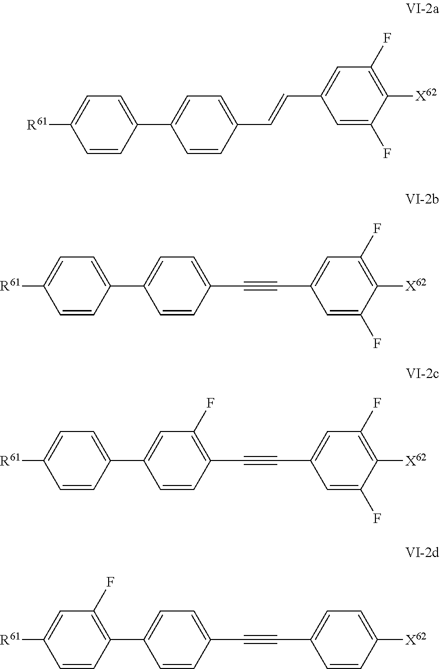

in which the parameters have the meaning given above under formula VI-2 and preferably
R⁶¹ has the meaning indicated above and preferably denotes CₙH₂ₙ₊₁, in which
n denotes an integer in the range from 0 to 7, preferably in the range from 1 to 5, and
X⁶² denotes —F, —Cl, —OCF₃, or —CN.

The compounds of the formula VI-3 are preferably selected from compounds of the formulae VI-3a to VI-3c:

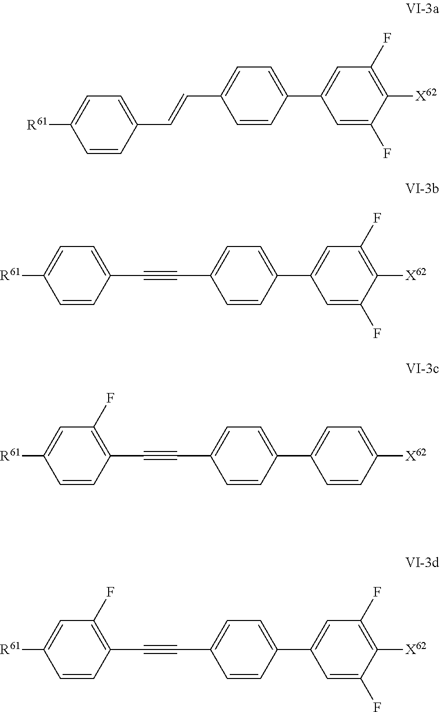

in which the parameters have the meaning given above under formula VI-3 and preferably
R⁶¹ has the meaning indicated above and preferably denotes CₙH₂ₙ₊₁, in which
n denotes an integer in the range from 0 to 7, preferably in the range from 1 to 5, and
X⁶² denotes F, Cl, OCF₃, or —CN.

The compounds of the formula VI-5 are preferably selected from the compounds of the formula VI-5b:

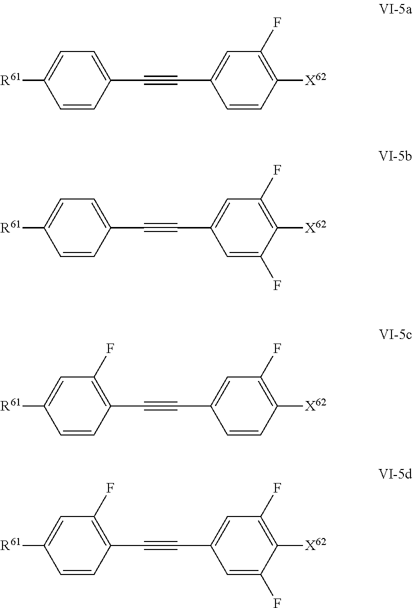

in which the parameters have the meaning given above under formula VI-5 and preferably
R⁶¹ has the meaning indicated above and preferably denotes CₙH₂ₙ₊₁, in which
n denotes an integer in the range from 0 to 7, preferably in the range from 1 to 5, and
X⁶² denotes —F, —Cl, —OCF₃, or —CN, particularly preferably —OCF₃.

The compounds of the formula VII are preferably selected from the group of the compounds of the formulae VII-1 to VII-6:

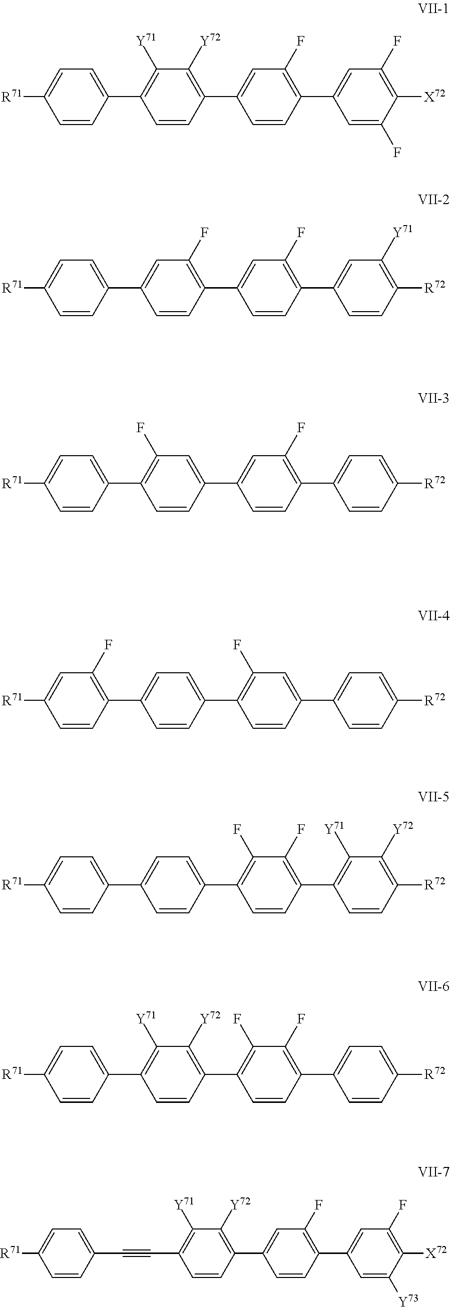

where the compounds of the formula VII-5 are excluded from the compounds of the formula VII-6, and

in which the parameters have the respective meanings indicated above for formula VII,
Y⁷¹, Y⁷², Y⁷³ independently from one another, denote H or F,

and preferably
R⁷¹ denotes alkyl or alkoxy, each having 1 to 7 C atoms, or alkenyl having 2 to 7 C atoms,
R⁷² denotes alkyl or alkoxy, each having 1 to 7 C atoms, or alkenyl having 2 to 7 C atoms,
X⁷² denotes F, Cl, NCS or —OCF₃, preferably F or NCS, and

particularly preferably
R⁷¹ has the meaning indicated above and preferably denotes CₙH₂ₙ₊₁ or CH₂═CH—(CH₂)z, and
R⁷² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The compounds of the formula VII-1 are preferably selected from the group of the compounds of the formulae VII-1a to VII-1d:

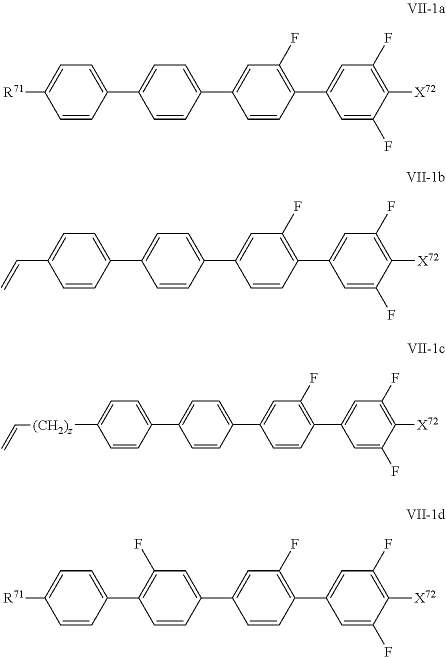

in which X⁷² has the meaning given above for formula VII-2 and
R⁷¹ has the meaning indicated above and preferably denotes CₙH₂ₙ₊₁, in which
n denotes 1 to 7, preferably 2 to 6, particularly preferably 2, 3 or 5, and
z denotes 0, 1, 2, 3 or 4, preferably 0 or 2, and
X⁷² preferably denotes F.

The compounds of the formula VII-2 are preferably selected from the group of the compounds of the formulae VII-2a and VII-2b, particularly preferably of the formula VII-2a:

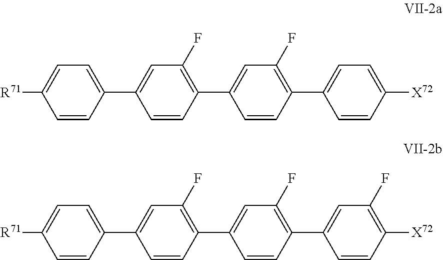

in which
R⁷¹ has the meaning indicated above and preferably denotes CₙH₂ₙ₊₁ or CH₂═CH—(CH₂)z, and
R⁷² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁷¹ and R⁷²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VII-3 are preferably compounds of the formula VII-3a:

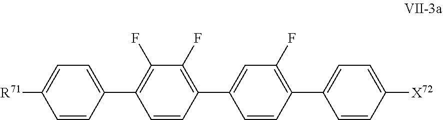

in which
R⁷¹ has the meaning indicated above and preferably denotes CₙH₂ₙ₊₁ or CH₂═CH—(CH₂)z, and
R⁷² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁷¹ and R⁷²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VII-4 are preferably compounds of the formula VII-4a:

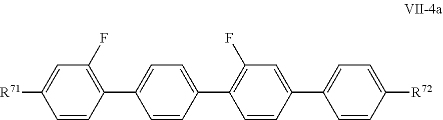

in which
R⁷¹ has the meaning indicated above and preferably denotes CₙH₂ₙ₊₁ or CH₂═CH—(CH₂)z, and
R⁷² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁷¹ and R⁷²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VII-5 are preferably selected from the group of the compounds of the formulae VII-5a and VII-5b, more preferably of the formula VII-5a:

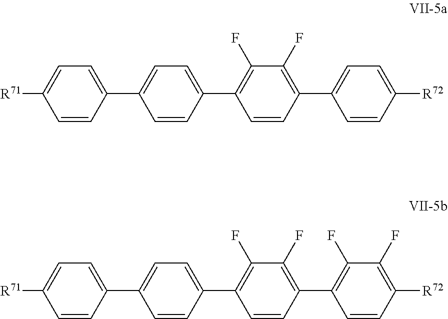

in which
R⁷¹ has the meaning indicated above and preferably denotes CₙH₂ₙ₊₁ or CH₂═CH—(CH₂)z, and
R⁷² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁷¹ and R⁷²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VII-6 are preferably selected from the group of the compounds of the formulae VII-6a and VII-6b:

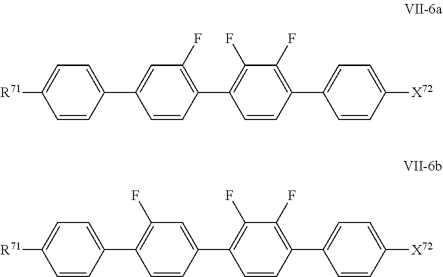

in which
R⁷¹ has the meaning indicated above and preferably denotes CₙH₂ₙ₊₁ or CH₂═CH—(CH₂)z, and
R⁷² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁷¹ and R⁷²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VII-7 are preferably selected from the group of the compounds of the formulae VII-7a and VII-7b:

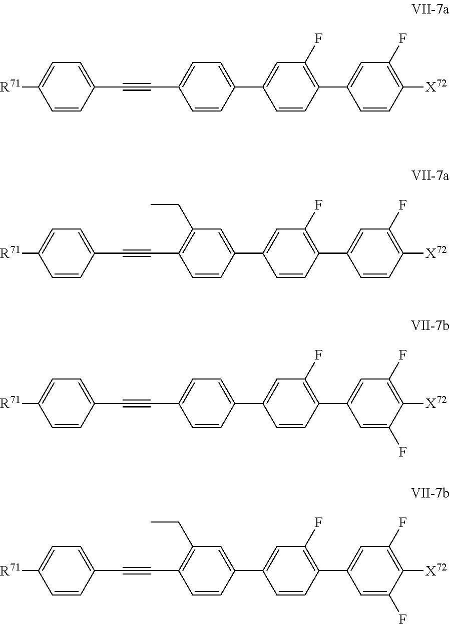

in which
R⁷¹ has the meaning indicated above and preferably denotes CₙH₂ₙ₊₁ or CH₂═CH—(CH₂)z,
X⁷² denotes F, —OCF₃ or —NCS,
n denotes an integer in the range from 0 to 15, preferably in the range from 1 to 7 and particularly preferably 1 to 5, and,
z denotes 0, 1, 2, 3 or 4, preferably 0 or 2.

The compounds of the formula VIII are preferably selected from the group of the compounds of the formulae VIII-1 to VIII-3, more preferably these compounds of the formula VIII predominantly consist, even more preferably essentially consist and very particularly preferably completely consist thereof:

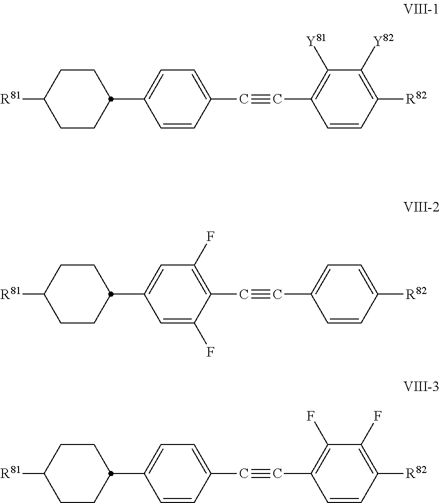

in which

one of
Y⁸¹ and Y⁸² denotes H and the other denotes H or F, and
R⁸¹ has the meaning indicated above and preferably denotes CₙH₂ₙ₊₁ or CH₂═CH—(CH₂)z, and
R⁸² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁸¹ and R⁸²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VIII-1 are preferably selected from the group of the compounds of the formulae VIII-1a to VIII-1c:

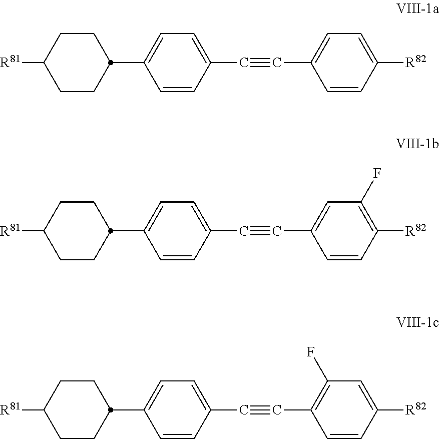

in which
R⁸¹ has the meaning indicated above and preferably denotes CₙH₂ₙ₊₁ or CH₂═CH—(CH₂)z, and
R⁸² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁸¹ and R⁸²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and CₘH₂ₘ₊₁).

The compounds of the formula VIII-2 are preferably compounds of the formula VII-2a:

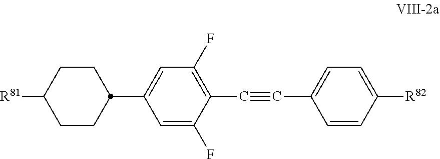

in which
R⁸¹ has the meaning indicated above and preferably denotes CₙH₂ₙ₊₁ or CH₂═CH—(CH₂)z, and
R⁸² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁸¹ and R⁸²) here are, in particular, (CₙH₂ₙ₊₁ and CₘH₂ₘ₊₁), (CₙH₂ₙ₊₁ and O—CₘH₂ₘ₊₁) and (CH₂═CH—(CH₂)z and CₘH₂ₘ₊₁), particularly preferably (CₙH₂ₙ₊₁ and CₘH₂ₘ₊₁).

The compounds of the formula VIII-3 are preferably compounds of the formula VII-3a:

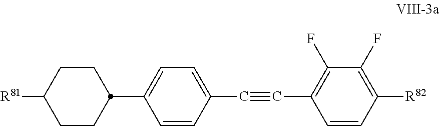

in which
R⁸¹ has the meaning indicated above and preferably denotes CₙH₂ₙ₊₁ or CH₂═CH—(CH₂)z, and
R⁸² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁸¹ and R⁸²) here are, in particular, (CₙH₂ₙ₊₁ and CₘH₂ₘ₊₁) and (CₙH₂ₙ₊₁ and O—CₘH₂ₘ₊₁).

The compounds of the formula IX are preferably selected from the group of the compounds of the formulae IX-1 to IX-3:

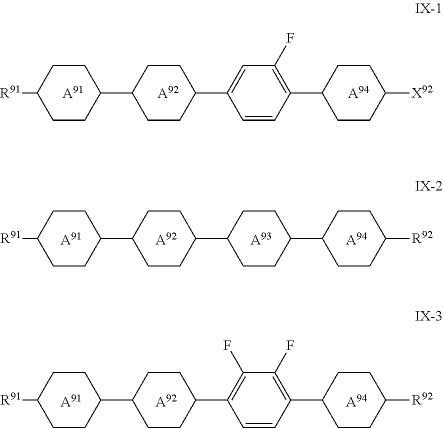

in which the parameters have the respective meaning indicated above under formula IX and preferably

one of

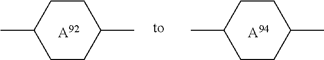

denotes

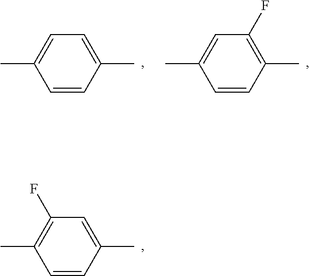

and

in which
R⁹¹ has the meaning indicated above and preferably denotes CₙH₂ₙ₊₁ or CH₂═CH—(CH₂)z, and
R⁹² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁹¹ and R⁹²) here are, in particular, (CₙH₂ₙ₊₁ and CₘH₂ₘ₊₁) and (CₙH₂ₙ₊₁ and O—CₘH₂ₘ₊₁).

The compounds of the formula IX-1 are preferably selected from the group of the compounds of the formulae IX-1a to IX-1e:

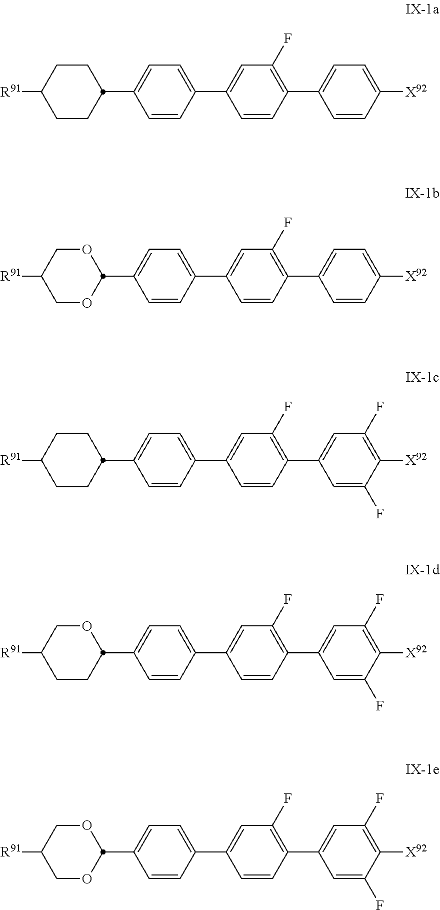

in which the parameters have the meaning given above and preferably
R⁹¹ has the meaning indicated above and preferably denotes CₙH₂ₙ₊₁, and
n denotes an integer in the range from 0 to 15, preferably in the range from 1 to 7 and particularly preferably 1 to 5, and
X⁹² preferably denotes F or Cl.

The compounds of the formula IX-2 are preferably selected from the group of the compounds of the formulae IX-2a and IX-2b:

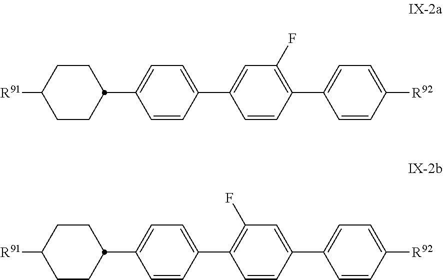

in which
R⁹¹ has the meaning indicated above and preferably denotes CₙH₂ₙ₊₁ or CH₂═CH—(CH₂)z, and
R⁹² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combination of (R⁹¹ and R⁹²) here is, in particular, (CₙH₂ₙ₊₁ and CₘH₂ₘ₊₁).

The compounds of the formula IX-3 are preferably compounds of the formulae IX-3a and IX-3b:

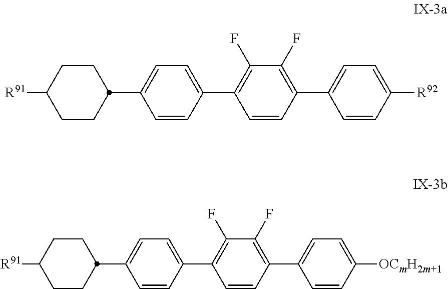

in which
R⁹¹ has the meaning indicated above and preferably denotes CₙH₂ₙ₊₁ or CH₂═CH—(CH₂)z, and
R⁹² has the meaning indicated above and preferably denotes CₘH₂ₘ₊₁ or O—CₘH₂ₘ₊₁ or (CH₂)z-CH═CH₂, and in which
n and m, independently of one another, denote an integer in the range from 0 to 15, preferably in the range from 1 to 7 and particularly preferably 1 to 5, and
z denotes 0, 1, 2, 3 or 4, preferably 0 or 2.

The preferred combinations of (R⁹¹ and R⁹²) here are, in particular, (CₙH₂ₙ₊₁ and CₘH₂ₘ₊₁) and (CₙH₂ₙ₊₁ and O—CₘH₂ₘ₊₁), particularly preferably (CₙH₂ₙ₊₁ and O—CₘH₂ₘ₊₁).

The media according to the present invention comprise one or more chiral dopants. Preferably these chiral dopants have an absolute value of the helical twisting power (HTP) in the range of from 1 μm⁻¹ to 150 μm⁻¹, preferably in the range of from 10 μm⁻¹ to 100 μm⁻¹. In case the media comprise two or more chiral dopants, these may have opposite signs of their HTP-values. This condition is preferred for some specific embodiments, as it allows to compensate the chirality of the respective compounds to some degree and, thus, may be used to compensate various temperature dependent properties of the resulting media in the devices. Generally, however, it is preferred that most, preferably all of the chiral compounds present in the media according to the present invention have the same sign of their HTP-values.

Preferably the chiral dopants present in the media according to the instant application are mesogenic compounds and most preferably they exhibit a mesophase on their own.

In a preferred embodiment of the present invention, the medium comprises two or more chiral compounds which all have the same algebraic sign of the HTP.

The temperature dependence of the HTP of the individual compounds may be high or low. The temperature dependence of the pitch of the medium can be compensated by mixing compounds having different temperature dependencies of the HTP in corresponding ratios.

For the optically active component, a multitude of chiral dopants, some of which are commercially available, is available to the person skilled in the art, such as, for example, cholesteryl nonanoate, R- and S-811, R- and S-1011, R- and S-2011, R- and S-3011, R- and S-4011, or CB15 (all Merck KGaA, Darmstadt).

Particularly suitable dopants are compounds which contain one or more chiral groups and one or more mesogenic groups, or one or more aromatic or alicyclic groups which form a mesogenic group with the chiral group.

Suitable chiral groups are, for example, chiral branched hydrocarbon radicals, chiral ethane diols, binaphthols or dioxolanes, furthermore mono- or polyvalent chiral groups selected from the group consisting of sugar derivatives, sugar alcohols, sugar acids, lactic acids, chiral substituted glycols, steroid derivatives, terpene derivatives, amino acids or sequences of a few, preferably 1-5, amino acids.

Preferred chiral groups are sugar derivatives, such as glucose, mannose, galactose, fructose, arabinose and dextrose, sugar alcohols, such as, for example, sorbitol, mannitol, iditol, galactitol or anhydro derivatives thereof, in particular dianhydrohexitols, such as dianhydrosorbide (1,4:3,6-dianhydro-D-sorbide, isosorbide), dianhydromannitol (isosorbitol) or dianhydroiditol (isoiditol), sugar acids, such as, for example, gluconic acid, gulonic acid and ketogulonic acid, chiral substituted glycol radicals, such as, for example, mono- or oligoethylene or propylene glycols, in which one or more CH₂ groups are substituted by alkyl or alkoxy, amino acids, such as, for example, alanine, valine, phenylglycine or phenylalanine, or sequences of from 1 to 5 of these amino acids, steroid derivatives, such as, for example, cholesteryl or cholic acid radicals, terpene derivatives, such as, for example, menthyl, neomenthyl, campheyl, pineyl, terpineyl, isolongifolyl, fenchyl, carreyl, myrthenyl, nopyl, geraniyl, linaloyl, neryl, citronellyl or dihydrocitronellyl.

The media according to the present invention preferably comprise chiral dopants which are selected from the group of known chiral dopants. Suitable chiral groups and mesogenic chiral compounds are described, for example, in DE 34 25 503, DE 35 34 777, DE 35 34 778, DE 35 34 779 and DE 35 34 780, DE 43 42 280, EP 01 038 941 and DE 195 41 820. Examples are also compounds listed in Table F below.

Chiral compounds preferably used according to the present invention are selected from the group consisting of the formulae shown below.

Particular preference is given to chiral dopants selected from the group consisting of compounds of the following formulae A-I to A-III and Ch:

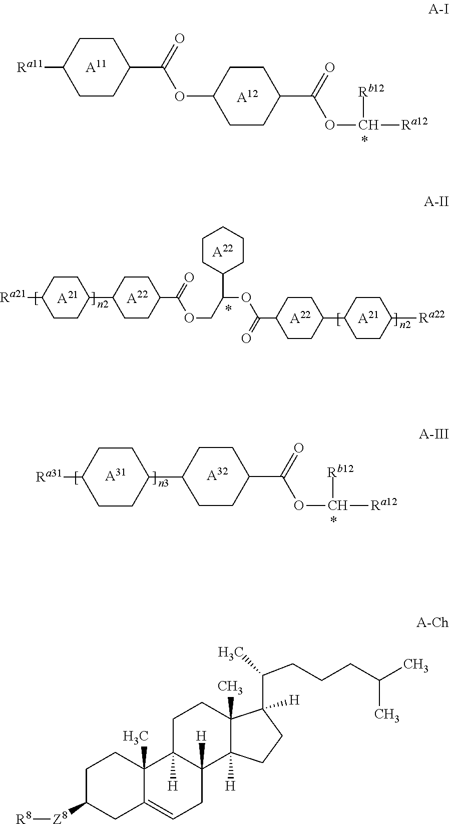

in which
R^{a11}, R^{a12} and R^{b12}, independently of one another, denote alkyl having 1 to 15 C atoms, in which, in addition, one or more non-adjacent CH₂ groups may each be replaced, independently of one another, by —C(R^{z})═C(R^{z})—, —C≡C—, —O—, —S—, —CO—, —CO—O—, —O—CO— or —O—CO—O— in such a way that O and/or S atoms are not linked directly to one another, and in which, in addition, one or more H atoms may be replaced by F, Cl, Br, I or CN, preferably alkyl, more preferably n-alkyl, with the proviso that R^{a12} is different from R^{b12}
R^{a21} and R^{a22}, independently of one another, denote alkyl having 1 to 15 C atoms, in which, in addition, one or more non-adjacent CH₂ groups may each be replaced, independently of one another, by —C(R^{z})═C(R^{z})—, —C≡C—, —O—, —S—, —CO—, —CO—O—, —O—CO— or —O—CO—O— in such a way that O and/or S atoms are not linked directly to one another, and in which, in addition, one or more H atoms may be replaced by F, Cl, Br, I or CN, preferably both are alkyl, more preferably n-alkyl,
R^{a31}, R^{a31} and R^{b32}, independently of one another, denote straight-chain or branched alkyl having 1 to 15 C atoms, in which, in addition, one or more non-adjacent CH₂ groups may each be replaced, independently of one another, by —C(R^{z})═C(R^{z})—, —C≡C—, —O—, —S—, —CO—, —CO—O—, —O—CO— or —O—CO—O— in such a way that O and/or S atoms are not linked directly to one another, and in which, in addition, one or more H atoms may be replaced by F, Cl, Br, I or CN,
   preferably alkyl, more preferably n-alkyl, with the proviso that R^{a32} is different from R^{b32},
R^{z} denotes H, CH₃, F, Cl, or CN, preferably H or F,
R⁸ has one of the meanings of R^{a11} given above, preferably alkyl, more preferably n-alkyl having 1 to 15 C atoms,
Z⁸ denotes —C(O)O—, —CH₂O—, —CF₂O— or a single bond, preferably —C(O)O—,
A¹¹ is defined as A¹² below, or alternatively denotes

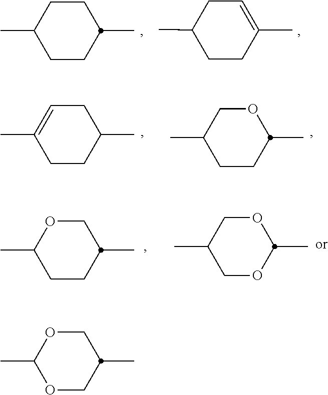

A¹² denotes

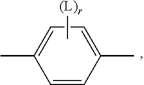

preferably

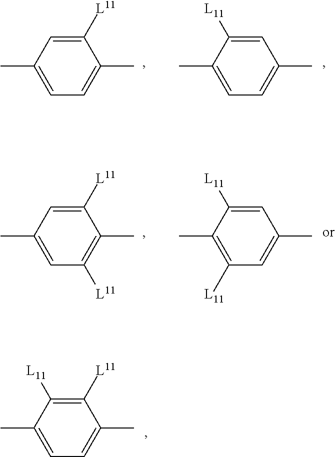

in which
L¹¹, on each occurrence, independently of one another, denotes halogen, CN, or alkyl, alkenyl, alkoxy or alkenyloxy having up to 12 C atoms and in which one or more H atoms are optionally replaced with halogen, preferably Me, Et, Cl or F, particularly preferably F.
A²¹ denotes

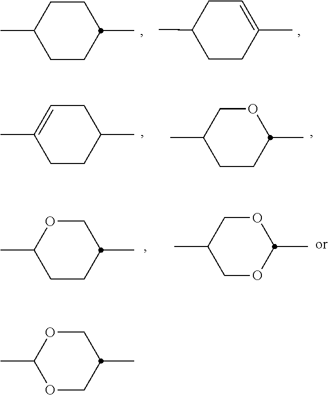

A²² has the meanings given for A¹²
A³¹ has the meanings given for A¹¹,
   alternatively denotes

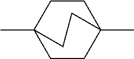

A³² has the meanings given for A¹².
n2 on each occurrence, identically or differently, is 0, 1 or 2, and
n3 is 1, 2 or 3.

Particular preference is given to dopants selected from the group consisting of the compounds of the following formulae:

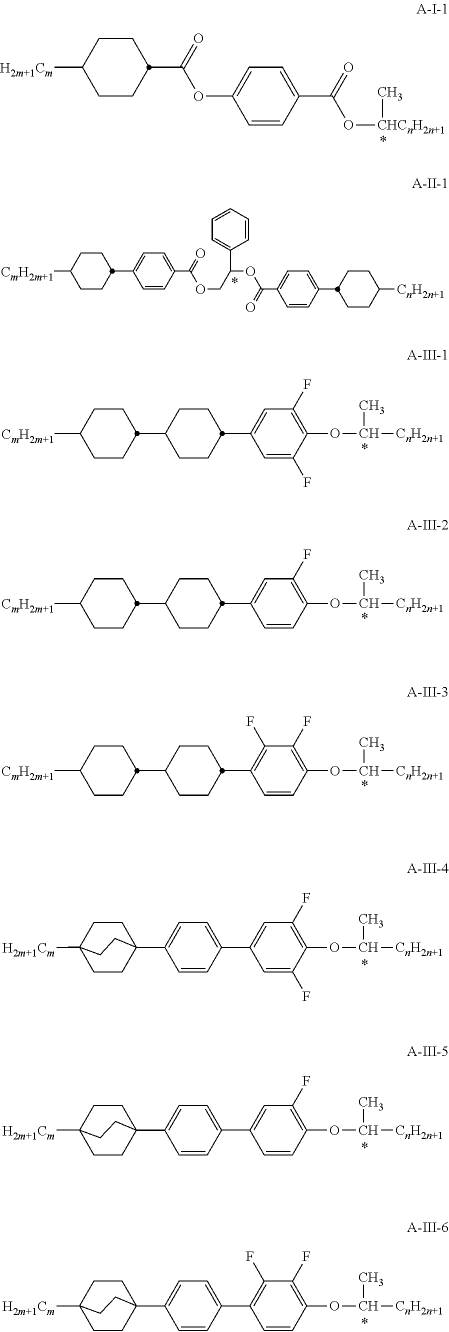

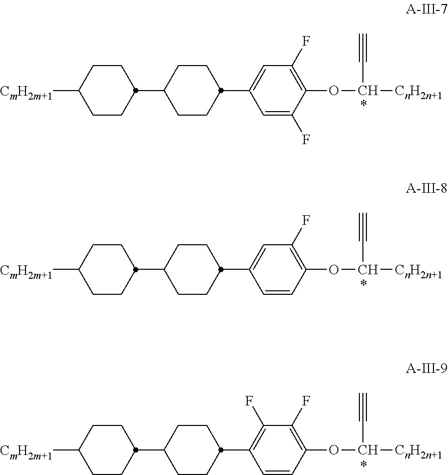

in which
m is, on each occurrence, identically or differently, an integer from 1 to 9 and
n is, on each occurrence, identically or differently, an integer from 2 to 9.

Particularly preferred compounds of formula A are compounds of formula A-III.

Further preferred dopants are derivatives of the isosorbide, isomannitol or isoiditol of the following formula A-IV:

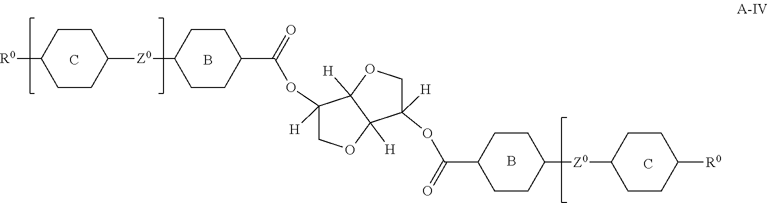

in which the group

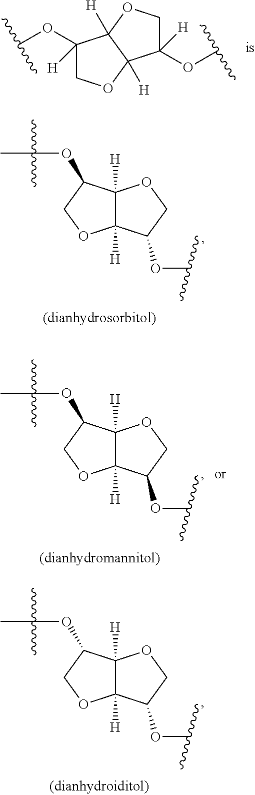 [SMILES: *OC1COC2C(O*)COC12 ; *O[C@H]1CO[C@H]2[C@@H]1OC[C@H]2O* ; *O[C@@H]1CO[C@H]2[C@@H]1OC[C@H]2O* ; *O[C@H]1CO[C@H]2[C@@H]1OC[C@@H]2O*]

preferably dianhydrosorbitol,

and chiral ethane diols, such as, for example, diphenylethanediol (hydrobenzoin), in particular mesogenic hydrobenzoin derivatives of the following formula A-V:

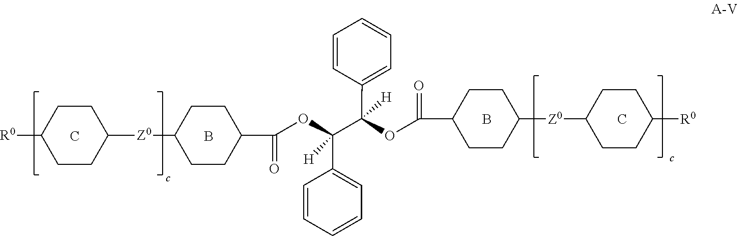

including the (S,S) enantiomers, which are not shown,

in which

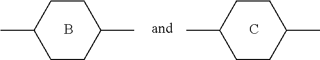

are each, independently of one another, 1,4-phenylene, which may also be mono-, di- or trisubstituted by L, or 1,4-cyclohexylene,
L is H, F, Cl, CN or optionally halogenated alkyl, alkoxy, alkylcarbonyl, alkoxycarbonyl or alkoxycarbonyloxy having 1-7 carbon atoms,
c is 0 or 1,
Z⁰ is —COO—, —OCO—, —CH₂CH₂— or a single bond, and
R⁰ is alkyl, alkoxy, alkylcarbonyl, alkoxycarbonyl or alkylcarbonyl-oxy having 1-12 carbon atoms.

Examples of compounds of formula A-IV are:

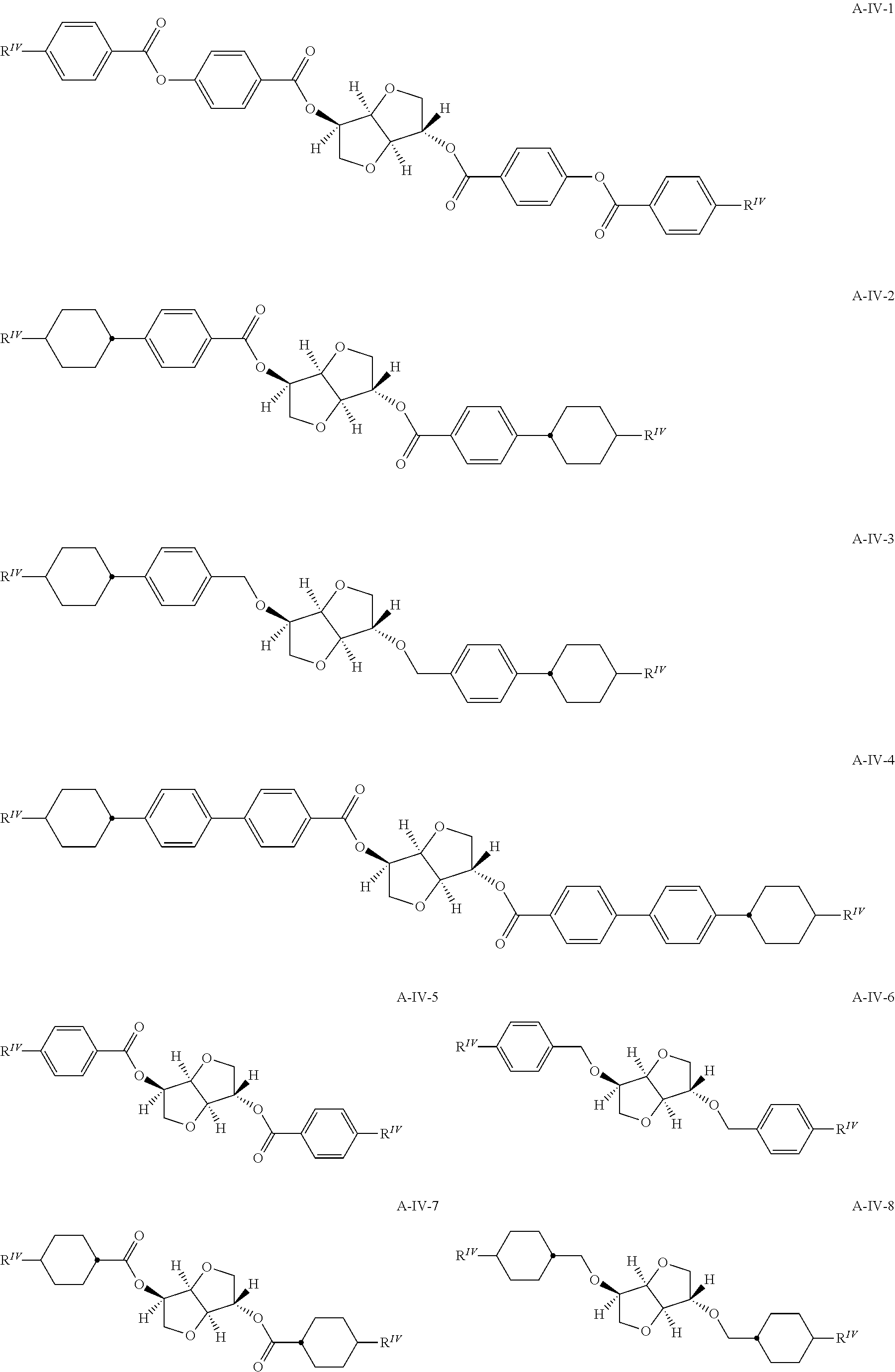

The compounds of the formula A-IV are described in WO 98/00428. The compounds of the formula A-V are described in GB-A-2,328,207.

Very particularly preferred dopants are chiral binaphthyl derivatives, as described in WO 02/94805, chiral binaphthol acetal derivatives, as described in WO 02/34739, chiral TADDOL derivatives, as described in WO 02/06265, and chiral dopants having at least one fluorinated bridging group and a terminal or central chiral group, as described in WO 02/06196 and WO 02/06195.

Particular preference is given to chiral compounds of the formula A-VI

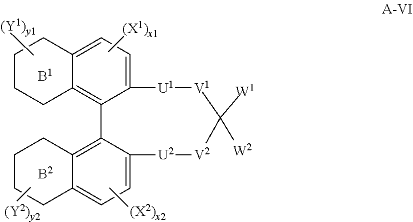

in which
X¹, X², Y¹ and Y² are each, independently of one another, F, Cl, Br, I, CN, SCN, SF₅, straight-chain or branched alkyl having from 1 to 25 carbon atoms, which may be monosubstituted or polysubstituted by F, Cl, Br, I or CN and in which, in addition, one or more non-adjacent CH₂ groups may each, independently of one another, be replaced by —O—, —S—, —NH—, NR⁰—, —CO—, —COO—, —OCO—, —OCOO—, —S—CO—, —CO—S—, —CH═CH— or —C≡C— in such a way that 0 and/or S atoms are not bonded directly to one another, a polymerisable group or cycloalkyl or aryl having up to 20 carbon atoms, which may optionally be monosubstituted or polysubstituted by halogen, preferably F, or by a polymerisable group,
x¹ and x² are each, independently of one another, 0, 1 or 2,
y¹ and y² are each, independently of one another, 0, 1, 2, 3 or 4,
B¹ and B² are each, independently of one another, an aromatic or partially or fully saturated aliphatic six-membered ring in which one or more CH groups may be replaced by N atoms and one or more non-adjacent CH₂ groups may be replaced by 0 and/or S,
W¹ and W² are each, independently of one another, —Z¹-A¹-(Z²-A²)ₘ-R, and one of the two is alternatively R¹ or A³, but both are not simultaneously H, or

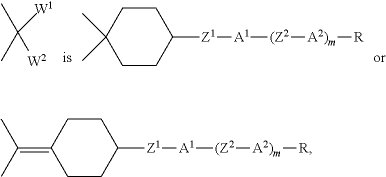

U¹ and U² are each, independently of one another, CH₂, O, S, CO or CS,
V¹ and V² are each, independently of one another, (CH₂)ₙ, in which from one to four non-adjacent CH₂ groups may be replaced by O and/or S, and one of V¹ and V² and, in the case where

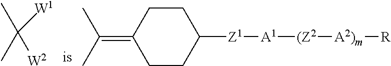

both are a single bond,
Z¹ and Z² are each, independently of one
   another, —O—, —S—, —CO—, —COO—, —OCO—, —O—COO—, —CO—NR⁰—, —NR⁰—CO—, —O—CH₂—, —CH₂—O—, —S—CH₂—, —CH₂—S—, —CF₂—O—, —O—CF₂—, —CF₂—S—, —S—CF₂—, —CH₂—CH₂—, —CF₂—CH₂—, —CH₂—CF₂—, —CF₂—
   CF₂—, —CH═N—, —N═CH—, —N═N—, —CH═CH—, —CF═CH—, —CH═CF—, —CF═CF—, —C≡C—, a combination of two of these groups, where no two O and/or S and/or N atoms are bonded directly to one another, preferably —CH═CH—COO—, or —COO—CH═CH—, or a single bond,
A¹, A² and A³ are each, independently of one another, 1,4-phenylene, in which one or two non-adjacent CH groups may be replaced by N, 1,4-cyclohexylene, in which one or two non-adjacent CH₂ groups may be replaced by 0 and/or S, 1,3-dioxolane-4,5-diyl, 1,4-cyclohexenylene, 1,4-bicyclo[2.2.2]octylene, piperidine-1,4-diyl, naphthalene-2,6-diyl, decahydronaphthalene-2,6-diyl or 1,2,3,4-tetrahydronaphthalene-2,6-diyl, where each of these groups may be monosubstituted or polysubstituted by L, and in addition A¹ is a single bond,
L is a halogen atom, preferably F, CN, NO₂, alkyl, alkoxy, alkylcarbonyl, alkoxycarbonyl or alkoxycarbonyloxy having 1-7 carbon atoms, in which one or more H atoms may be replaced by F or Cl,
m is in each case, independently, 0, 1, 2 or 3, and
R and R¹ are each, independently of one another, H, F, Cl, Br, I, CN, SCN, SF₅, straight-chain or branched alkyl having from 1 or 3 to 25 carbon atoms respectively, which may optionally be monosubstituted or polysubstituted by F, Cl, Br, I or CN, and in which one or more non-adjacent CH₂ groups may be replaced by —O—, —S—, —NH—, —NR⁰—, —CO—, —COO—, —OCO—, —O—COO—, —S—CO—, —CO—S—, —CH═CH— or —C≡C—, where no two O and/or S atoms are bonded directly to one another, or a polymerisable group.

Particular preference is given to chiral binaphthyl derivatives of the formula A-VI-1

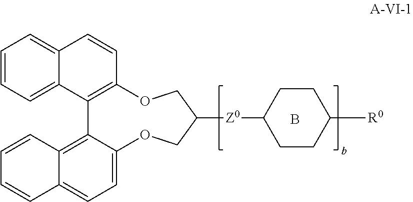

in particular those selected from the following formulae A-VI-1a to A-VI-1c:

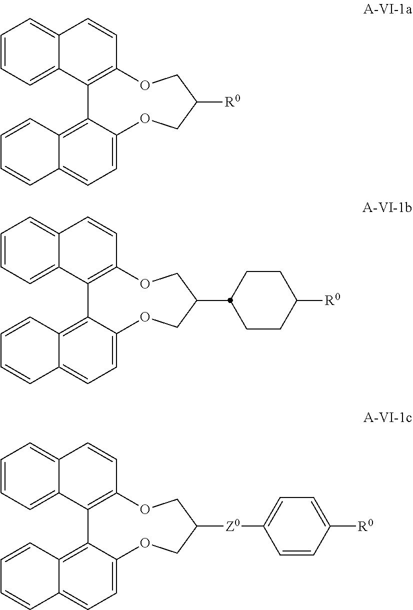

in which ring B and Z⁰ are as defined for the formula A-IV, and
R⁰ as defined for formula A-IV or H or alkyl having from 1 to 4 carbon atoms, and
b is 0, 1 or 2,
and Z⁰ is, in particular, —OC(O)— or a single bond.

The concentration of the one or more chiral dopant(s), in the LC medium is preferably in the range from 0.001% to 20%, preferably from 0.05% to 5%, more preferably from 0.1% to 2%, and, most preferably from 0.5% to 1.5%. These preferred concentration ranges apply in particular to the chiral dopant S-4011 or R-4011 (both from Merck KGaA) and for chiral dopants having the same or a similar HTP. For Chiral dopants having either a higher or a lower absolute value of the HTP compared to S-4011 these preferred concentrations have to be decreased, respectively increased proportionally according to the ratio of their HTP values relatively to that of S-4011.

The pitch p of the LC media or host mixtures according to the invention is preferably in the range of from 5 to 50 μm, more preferably from 8 to 30 μm and particularly preferably from 10 to 20 μm.

Preferably, the media according to the invention, comprise a stabilizer selected from the group of compounds of the formulae ST-1 to ST-18.

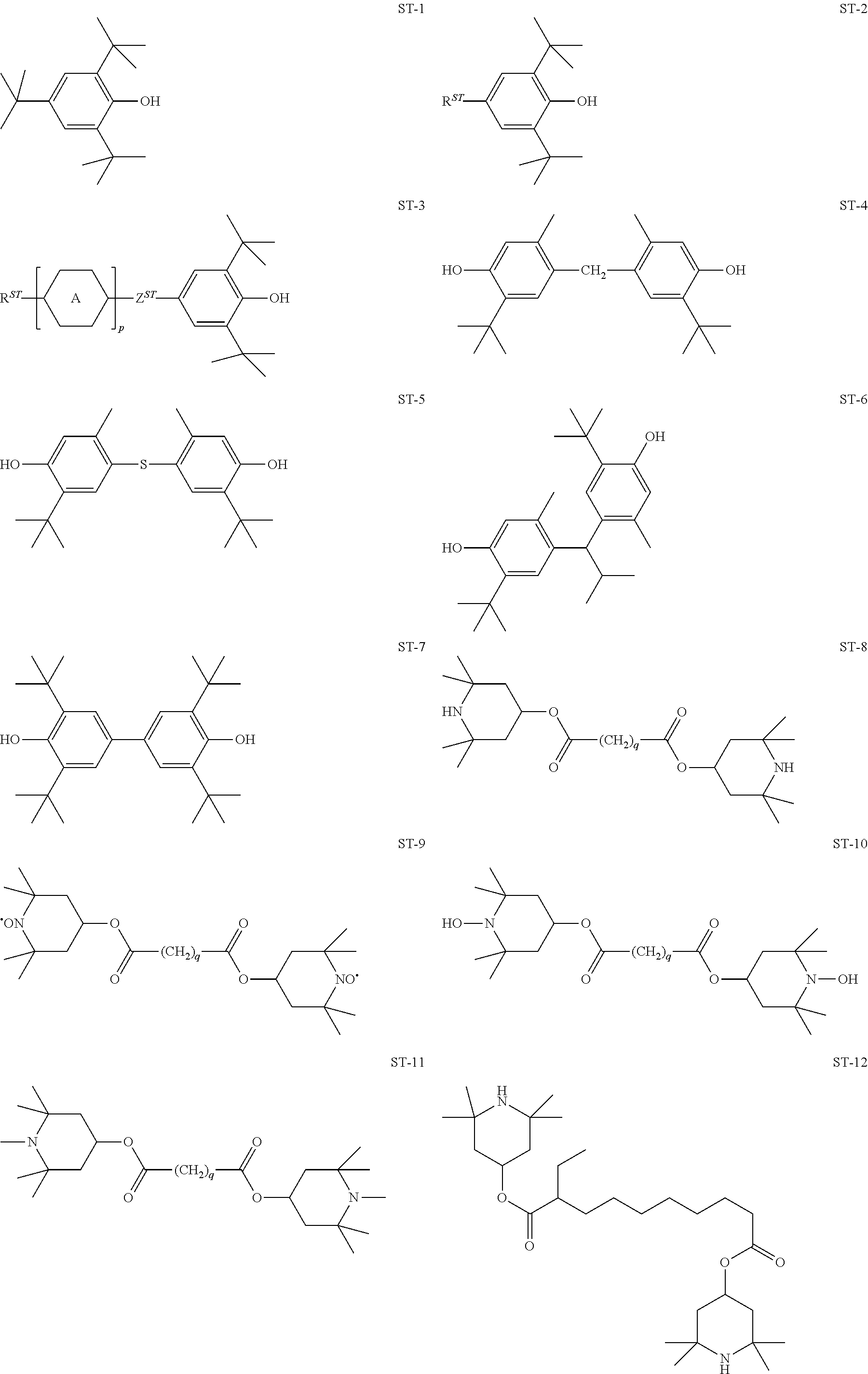

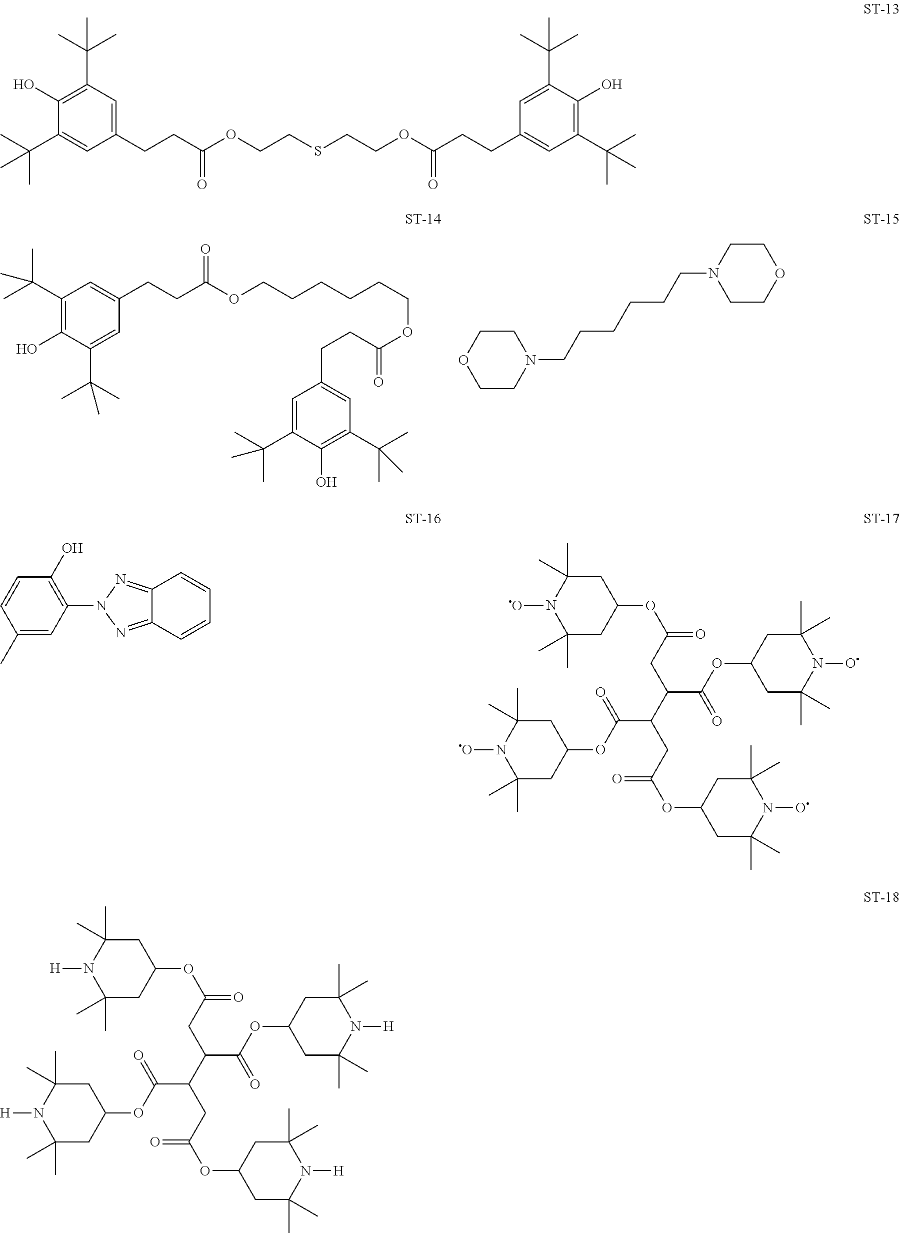

in which
R^{ST} denotes H, an alkyl or alkoxy radical having 1 to 15 C atoms, where, in addition, one or more CH₂ groups in these radicals may each be replaced, independently of one another, by —C≡C—, —CF₂O—, —OCF₂—, —CH═CH—,

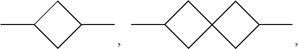

O—, —CO—O—, —O—CO— in such a way that O atoms are not linked directly to one another, and in which, in addition, one or more H atoms may be replaced by halogen,

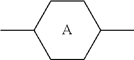

denotes

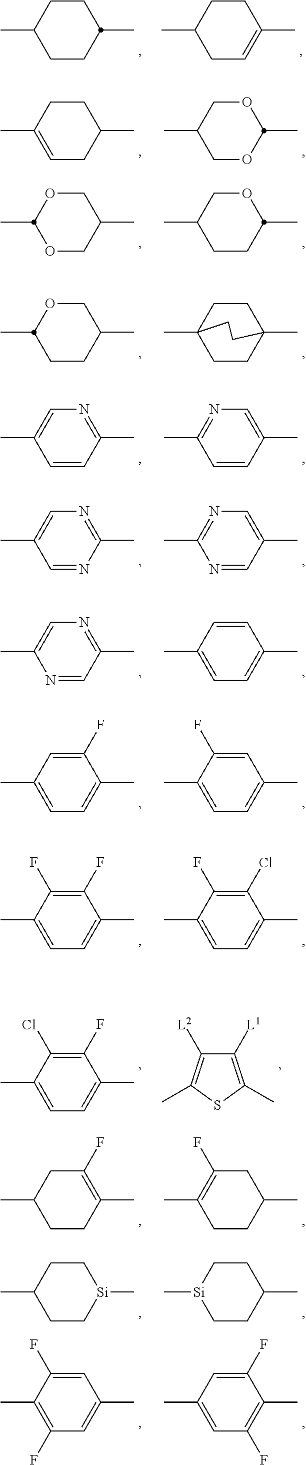

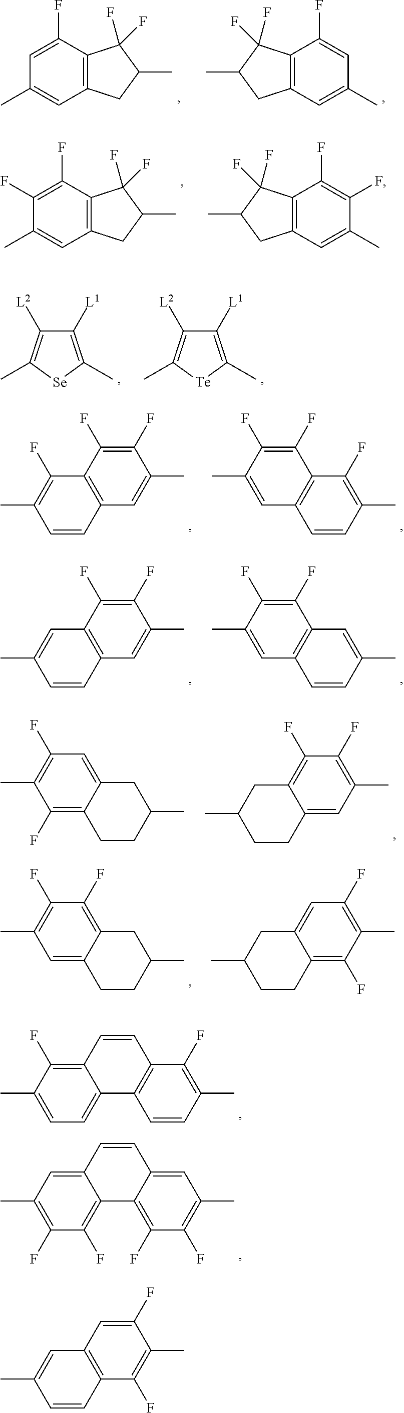

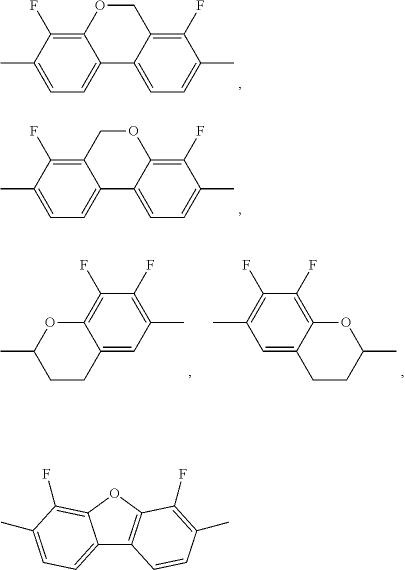

Z^{ST} each, independently of one another, denote —CO—O—, —O—CO—, —CF₂O—, —OCF₂—, —CH₂O—, —OCH₂—, —CH₂—, —CH₂CH₂—, —(CH₂)₄—, —CH═CH—CH₂O—, —C₂F₄—, —CH₂CF₂—, —CF₂CH₂—, —CF═CF—, —CH═CF—, —CF═CH—, —CH═CH—, —C≡C— or a single bond,
L¹ and L² each, independently of one another, denote F, Cl, CF₃ or CHF₂,
p denotes 1 or 2,
q denotes 1, 2, 3, 4, 5, 6, 7, 8, 9 or 10.

Of the compounds of the formula ST, special preference is given to the compounds of the formulae

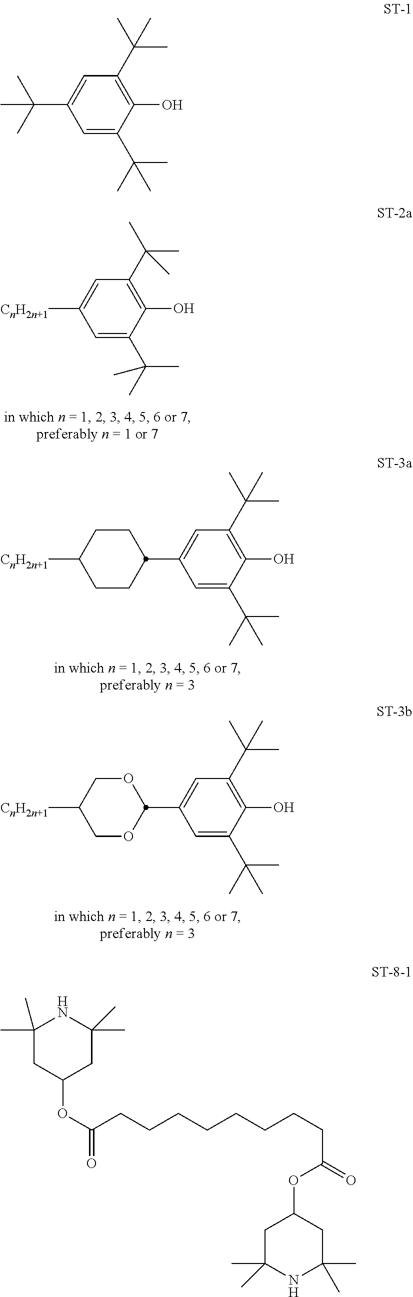

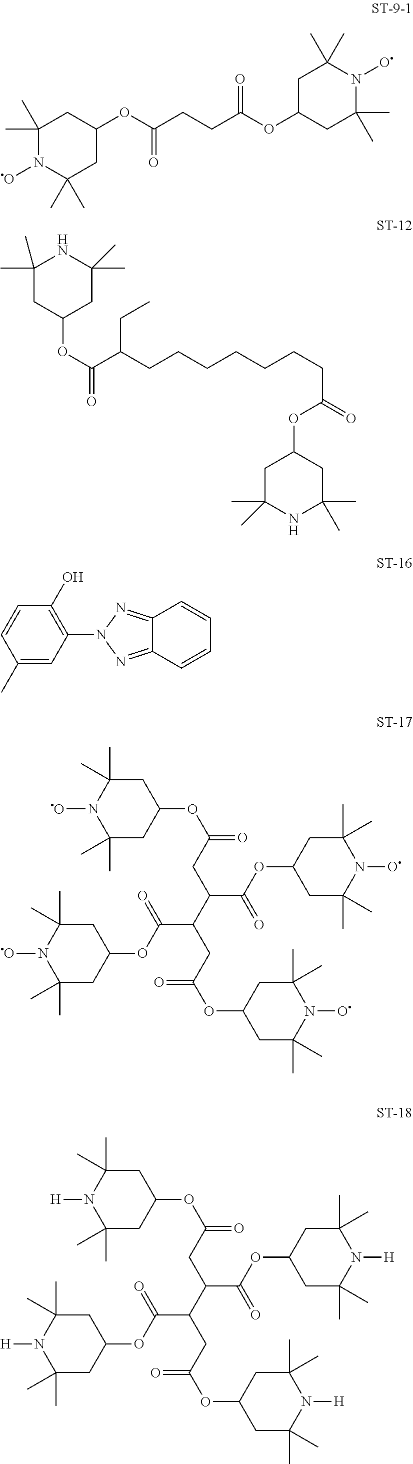

In the compounds of the formulae ST-3a and ST-3b, n preferably denotes 3. In the compounds of the formula ST-2a, n preferably denotes 7.

Very particularly preferred mixtures according to the invention comprise one or more stabilisers from the group of the compounds of the formulae ST-2a-1, ST-3a-1, ST-3b-1, ST-8-1, ST-9-1 and ST-12:

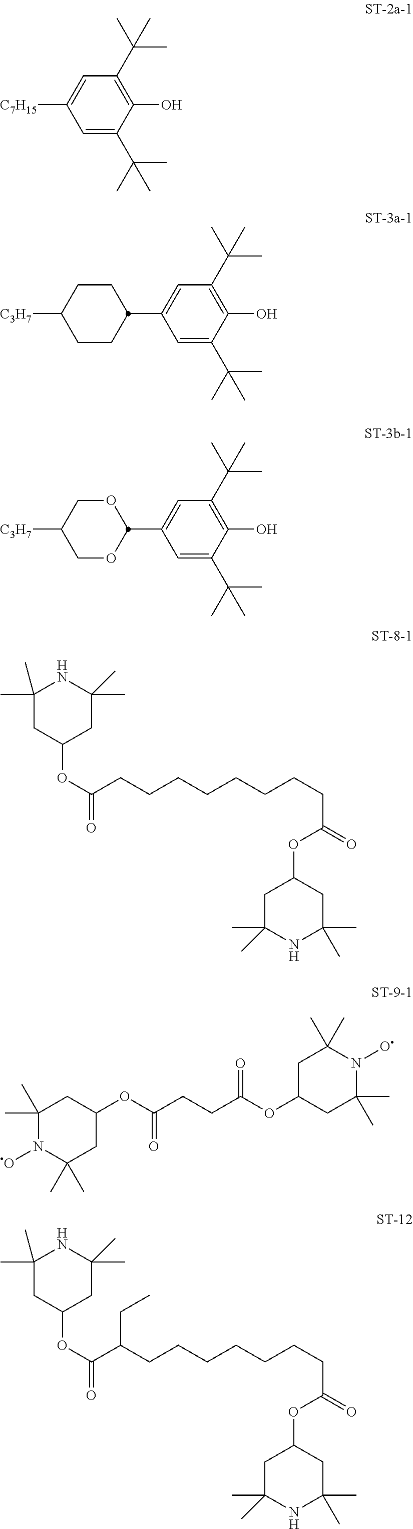

The compounds of the formulae ST-1 to ST-18 are preferably each present in the liquid-crystal mixtures according to the invention in amounts of 0.005-0.5%, based on the mixture.

If the mixtures according to the invention comprise two or more compounds from the group of the compounds of the formulae ST-1 to ST-18, the concentration correspondingly increases to 0.01-1% in the case of two compounds, based on the mixtures.

However, the total proportion of the compounds of the formulae ST-1 to ST-18, based on the mixture according to the invention, should not exceed 2%.

The compounds according to the present invention can be synthesized by or in analogy to known methods described in the literature (for example in the standard works such as Houben-Weyl, Methoden der Organischen Chemie [Methods of Organic Chemistry], Georg-Thieme-Verlag, Stuttgart), under reaction conditions which are known and suitable for said reactions. Use may also be made here of variants which are known per se, but are not mentioned here. In particular, they can be prepared as described in or in analogy to the following reaction schemes. Further methods for preparing the inventive compounds can be taken from the examples.

The compounds of formula S are preferably synthesized following or in analogy to the procedures described in CN 106518890 A.

Other mesogenic compounds which are not explicitly mentioned above can optionally and advantageously also be used in the media in accordance with the present invention. Such compounds are known to the person skilled in the art.

Preferably, the total concentration of the one or more compounds of formula S in the medium is in the range of from 1% to 50%, preferably from 5% to 40% and particularly preferably from 10% to 20%.

In another embodiment of the present invention, the medium comprises one or more compounds of formula S and one or more compounds of formula IC, where the total concentration of the one or more compounds of formula IC is in the range of from 5% to 50%, more preferably from 10% to 40%, and particularly preferably from 15% to 35%.

In a preferred embodiment of the present invention, the liquid-crystalline media preferably comprise in total 5% to 35%, preferably 10% to 32% and particularly preferably 20% to 30% of compounds of formula T.

In a preferred embodiment of the present invention, the liquid-crystalline medium comprises in total 30% or less, preferably 15% or less and particularly preferably 10% or less compounds of formula T.

In a preferred embodiment of the present invention, the liquid-crystalline medium comprises in total 30% or more, preferably 40% or more and particularly preferably 50% or more compounds of formula I and/or IC, preferably selected from the group of compounds of the I-1, I-2 and IC, particularly preferably selected from the compounds of the formulae I-2 and IC.

Preferably, the proportion of the compounds if formula I-1 in the medium is 20% or less, more preferably 15% or less, particularly preferably 10% or less and very particularly preferably 5% or less.

In a preferred embodiment, the medium comprises one or more compounds of formula I-2 in a total concentration in the range of from 5 to 25%, more preferably from 7% to 25%, and particularly preferably from 10% to 20%.

In another preferred embodiment, the medium comprises one or more compounds of formula I-2 in a total concentration of 10% or less, preferable of 5% or less and particularly preferably of 2% or less.

In a preferred embodiment, the total concentration of the compounds of formula IC in the media according to the present invention is in the range of from 5% to 50%, more preferably from 10% to 40%, and particularly preferably from 15% to 35%.

In a preferred embodiment, the total concentration of the compounds of formula IC in the media according to the present invention is 20% or more, more preferably 25% or more and particularly preferably 30% or more.

In a preferred embodiment of the present invention the medium comprises one or more compounds of formula II and/or IIA-1, preferably 11-1 and/or II-1-A1, in a total concentration of 5% to 35%, more preferably 10% to 30%, particularly preferably 15% to 25%.

Preferably, the medium comprises one or more the compounds of the formula P(2)TU-n-S.

Preferably, the medium comprises one or more the compounds of the formula II-1.

Preferably, the medium comprises either one or more the compounds of the formula P(2)TU-n-S or one or more compounds of the formula II-1.

In a preferred embodiment of the present invention the medium comprises one or more compounds of formula II-1 in an total concentration of 25% or less, more preferably 20% or less, particularly preferably 15% or less, very particularly preferably 10% or less.

In a preferred embodiment of the present invention the medium comprises one or more compounds of formula III, preferably III-1, in a total concentration of 2% to 30%, more preferably 5% to 25%, particularly preferably 10% to 20%.

Further preferred embodiments of the present invention, taken alone or in combination with one another, are as follows, wherein some compounds are abbreviated using the acronyms given in Table C:
The medium comprises one or more compounds of formula S and one or more compounds of formula IC
The medium comprises one or more compounds of formula T-1;
The medium comprises one or more compounds of formula T-2;
The medium comprises one or more compounds of formula T-1 and T-2;
The medium comprises one or more compounds of formula T-1 and/or T-2 and one or more compounds of formula I and/or IC and/or II and/or III;
The medium comprises one or more compounds of formula III-1;
The medium comprises two or more compounds of formula T-1;
The medium comprises two or more compounds of formula T-2;
The medium comprises the compound PPU-TO-S;
The medium comprises one or more compounds of formula S and one or more compounds of formula III-1, preferably in a total concentration in the range of from 5 to 40%, more preferably from 10 to 35%, particularly preferably from 18 to 30%;
The medium comprises one or more compounds of formula S and one or more compounds of formula IC, preferably in a total concentration in the range of from 20 to 65%, more preferably from 30 to 60%, particularly preferably from 45 to 55%;
The medium comprises one or more compounds of formula ICb, preferably the compounds CPU-2-S and/or CPU-4-S;
The medium comprises one or more compounds of formula ICb and formula II-1 b, preferably the compounds CPU-2-S and/or CPU-4-S and PTU-3-S and/or PTU-4-S and/or PTU-5-S;
The medium comprises one or more compounds of formula PPTU-n-S in an total concentration in the range of from 5 to 15%;
The medium comprises one or more compounds of formula PPTU-n-S and PGTU-n-S in an total concentration in the range of from 10 to 20%;
The medium comprises one or more compounds of formula ST-3, preferably ST-3a and/or ST-3b, particularly preferably ST-3b-1, in a total concentration in the range of from 0.01 to 1%, preferably from 0.05 to 0.5%, particularly from 0.10 to 0.15%.

The liquid-crystal media in accordance with the present invention preferably have a clearing point of 90° C. or more, more preferably 100° C. or more, more preferably 110° C. or more, more preferably 120° C. or more, more preferably 130° C. or more, particularly preferably 140° C. or more and very particularly preferably 150° C. or more.

The liquid-crystal media in accordance with the present invention preferably have a clearing point of 210° C. or less, more preferably 200° C. or less, particularly preferably 190° C. or less, and very particularly preferably 180° C. or less.

The nematic phase of the media according to the invention preferably extends at least from 0° C. or less to 90° C. or more. It is advantageous for the media according to the invention to exhibit even broader nematic phase ranges, preferably at least from −10° C. or less to 120° C. or more, very preferably at least from −20° C. or less to 140° C. or more and in particular at least from −30° C. or less to 150° C. or more, very particularly preferably at least from −40° C. or less to 170° C. or more.

The Δε of the liquid-crystal medium according to the present invention, at 1 kHz and 20° C., is preferably 5 or more, more preferably 10 or more and very preferably 12 or more.

The birefringence (Δn) of the liquid-crystal media according to the present invention, at 589 nm (Na^{D}) and 20° C., is preferably in the range of from 0.250 to 0.900, more preferably from 0.300 to 0.850, and very particularly preferably in the range from 0.350 to 0.800 or less.

The birefringence (Δn) of the liquid-crystal media according to the present invention, at 589 nm (Na^{D}) and 20° C., is preferably 0.250 or more, more preferably 0.300 or more, and particularly preferably 0.35 or more.

The compounds of the formulae I to III in each case include dielectrically positive compounds having a dielectric anisotropy of greater than 3, dielectrically neutral compounds having a dielectric anisotropy of less than 3 and greater than −1.5 and dielectrically negative compounds having a dielectric anisotropy of −1.5 or less.

The compounds of the formulae I, II and III are preferably dielectrically positive.

Herein, the expression dielectrically positive describes compounds or components where Δε>3.0, dielectrically neutral describes those where −1.5≤Δε≤3.0 and dielectrically negative describes those where Δε<−1.5. AE is determined at a frequency of 1 kHz and at 20° C. The dielectric anisotropy of the respective compound is determined from the results of a solution of 10% of the respective individual compound in a nematic host mixture. If the solubility of the respective compound in the host mixture is less than 10%, the concentration is reduced to 5%. The capacitances of the test mixtures are determined both in a cell having homeotropic alignment and in a cell having homogeneous alignment. The cell thickness of both types of cells is approximately 20 μm. The voltage applied is a rectangular wave having a frequency of 1 kHz and an effective value of typically 0.5 V to 1.0 V, but it is always selected to be below the capacitive threshold of the respective test mixture.

Δε is defined as (ε∥−ε⊥), while cave is (ε∥+2 ε⊥)/3.

The host mixture used for dielectrically positive compounds is mixture ZLI-4792 and that used for dielectrically neutral and dielectrically negative compounds is mixture ZLI-3086, both from Merck KGaA, Germany. The absolute values of the dielectric constants of the compounds are determined from the change in the respective values of the host mixture on addition of the compounds of interest. The values are extrapolated to a concentration of the compounds of interest of 100%.

Components having a nematic phase at the measurement temperature of 20° C. are measured as such, all others are treated like compounds.

The expression threshold voltage in the present application refers to the optical threshold and is quoted for 10% relative contrast (V₁₀), and the expression saturation voltage refers to the optical saturation and is quoted for 90% relative contrast (V₉₀), in both cases unless expressly stated otherwise. The capacitive threshold voltage (V₀), also called the Freeder-icks threshold (V_{Fr}), is only used if expressly mentioned.

The parameter ranges indicated in this application all include the limit values, unless expressly stated otherwise.

The different upper and lower limit values indicated for various ranges of properties in combination with one another give rise to additional preferred ranges.

Herein, the following conditions and definitions apply, unless expressly stated otherwise. All concentrations are quoted in percent by weight and relate to the respective mixture as a whole, all temperatures are quoted in degrees Celsius and all temperature differences are quoted in differential degrees. All physical properties are determined in accordance with “Merck Liquid Crystals, Physical Properties of Liquid Crystals”, Status November 1997, Merck KGaA, Germany, and are quoted for a temperature of 20° C., unless expressly stated otherwise. The optical anisotropy (Δn) is determined at a wavelength of 589.3 nm. The dielectric anisotropy (Δε) is determined at a frequency of 1 kHz. The threshold voltages, as well as all other electro-optical properties, are determined using test cells produced at Merck

KGaA, Germany. The test cells for the determination of Δε have a cell thickness of approximately 20 μm. The electrode is a circular ITO electrode having an area of 1.13 cm² and a guard ring. The orientation layers are SE-1211 from Nissan Chemicals, Japan, for homeotropic orientation (ε∥) and polyimide AL-1054 from Japan Synthetic Rubber, Japan, for homogeneous orientation (ε⊥). The capacitances are determined using a Solatron 1260 frequency response analyser using a sine wave with a voltage of 0.3 Vᵣₘₛ. The light used in the electro-optical measurements is white light. A set-up using a commercially available DMS instrument from Autronic-Melchers, Germany, is used here. The character-istic voltages have been determined under perpendicular observation. The threshold (V₁₀), mid-grey (V₅₀) and saturation (V₉₀) voltages have been determined for 10%, 50% and 90% relative contrast, respectively.

The liquid-crystalline media are investigated with respect to their properties in the microwave frequency range as described in A. Penirschke et al. “Cavity Perturbation Method for Characterization of Liquid Crystals up to 35 GHz”, 34^{th} European Microwave Conference—Amsterdam, pp. 545-548. Compare in this respect also A. Gaebler et al. “Direct Simulation of Material Permittivities . . . ”, 12MTC 2009—International Instrumentation and Measurement Technology Conference, Singapore, 2009 (IEEE), pp. 463-467, and DE 10 2004 029 429 A, in which a measurement method is likewise described in detail.

The liquid crystal is introduced into a polytetrafluoroethylene (PTFE) or quartz capillary. The capillary has an inner diameter of 0.5 mm and an outer diameter of 0.78 mm. The effective length is 2.0 cm. The filled capillary is introduced into the centre of the cylindrical cavity with a resonance frequency of 19 GHz. This cavity has a length of 11.5 mm and a radius of 6 mm. The input signal (source) is then applied, and the frequency depending response of the cavity is recorded using a commercial vector network analyser (N5227A PNA Microwave Network Analyzer, Keysight Technologies Inc. USA. For other frequencies, the dimensions of the cavity are adapted correspondingly.

The change in the resonance frequency and the Q factor between the measurement with the capillary filled with the liquid crystal and the measurement without the capillary filled with the liquid crystal is used to deter-mine the dielectric constant and the loss angle at the corresponding target frequency by means of equations 10 and 11 in the above-mentioned publi-cation A. Penirschke et al., 34th European Microwave Conference—Amsterdam, pp. 545-548, as described therein.

The values for the components of the properties perpendicular and parallel to the director of the liquid crystal are obtained by alignment of the liquid crystal in a magnetic field. To this end, the magnetic field of a permanent magnet is used. The strength of the magnetic field is 0.35 tesla.

Preferred components are phase shifters, varactors, wireless and radio wave antenna arrays, matching circuit adaptive filters and others.

Herein, the term “compounds” is taken to mean both one compound and a plurality of compounds, unless expressly stated otherwise.

The liquid-crystal media according to the invention preferably have nematic phases in preferred ranges given above. The expression have a nematic phase here means on the one hand that no smectic phase and no crystallisation are observed at low temperatures at the corresponding temperature and on the other hand that no clearing occurs on heating from the nematic phase. At high temperatures, the clearing point is measured in capillaries by conventional methods. The investigation at low temperatures is carried out in a flow viscometer at the corresponding temperature and checked by storage of bulk samples: The storage stability in the bulk (LTS) of the media according to the invention at a given temperature T is determined by visual inspection. 2 g of the media of interest are filled into a closed glass vessel (bottle) of appropriate size placed in a refrigerator at a predetermined temperature. The bottles are checked at defined time intervals for the occurrence of smectic phases or crystallisation. For every material and at each temperature two bottles are stored. If crystallisation or the appearance of a smectic phase is observed in at least one of the two correspondent bottles the test is terminated and the time of the last inspection before the one at which the occurrence of a higher ordered phase is observed is recorded as the respective storage stability. The test is finally terminated after 1000 h, i.e an LTS value of 1000 h means that the mixture is stable at the given temperature for at least 1000 h.

The liquid crystals employed preferably have a positive dielectric anisotropy. This is preferably 2 or more, preferably 4 or more, particularly preferably 6 or more and very particularly preferably 10 or more.

Furthermore, the liquid-crystal media according to the invention are characterised by high anisotropy values in the microwave range. The birefringence at about 19 GHz is, for example, preferably 0.14 or more, particularly preferably 0.15 or more, particularly preferably 0.20 or more, particularly preferably 0.25 or more and very particularly preferably 0.30 or more. In addition, the birefringence is preferably 0.80 or less.

The dielectric anisotropy in the microwave range is defined as

Δεᵣ=(ε_{r,∥}−ε_{r,⊥}).

The tunability (τ) is defined as

τ≡(Δεᵣ/ε_{r,∥}).

The material quality (η) is defined as

η≡(τ/tan δ_{εr,max})), where

the maximum dielectric loss is

tan δ_{ε r,max}≡max. {tan δε_{r,⊥};tan δε_{r,∥}}.

The material quality (η) of the preferred liquid-crystal materials is 6 or more, preferably 8 or more, preferably 10 or more, preferably 15 or more, preferably 17 or more, preferably 20 or more, particularly preferably 25 or more and very particularly preferably 30 or more.

In the corresponding components, the preferred liquid-crystal materials have phase shifter qualities of 15°/dB or more, preferably 20°/dB or more, preferably 30°/dB or more, preferably 40°/dB or more, preferably 50°/dB or more, particularly preferably 80°/dB or more and very particularly preferably 100°/dB or more.

In some embodiments, however, liquid crystals having a negative value of the dielectric anisotropy can also advantageously be used.

The liquid crystals employed are either individual substances or mixtures. They preferably have a nematic phase.

In the present application, high-frequency technology means applications of electromagnetic radiation having frequencies in the range of from 1 MHz to 1 THz, preferably from 1 GHz to 500 GHz, more preferably 2 GHz to 300 GHz, particularly preferably from about 5 GHz to 150 GHz.

Preferably, the devices according to the invention are operable in the microwave range.

The liquid-crystal media in accordance with the present invention may comprise further additives and chiral dopants in the usual concentrations. The total concentration of these further constituents is in the range from 0% to 10%, preferably 0.1% to 6%, based on the mixture as a whole. The concentrations of the individual compounds used are each preferably in the range from 0.1% to 3%. The concentration of these and similar additives is not taken into consideration when quoting the values and concentration ranges of the liquid-crystal components and liquid-crystal compounds of the liquid-crystal media in this application.

Preferably the media according to the present invention comprise one or more chiral compounds as chiral dopants in order to adjust their cholesteric pitch. Their total concentration in the media according to the instant invention is preferably in the range 0.05% to 15%, more preferably from 1% to 10% and most preferably from 2% to 6%.

Optionally the media according to the present invention may comprise further liquid crystal compounds in order to adjust the physical properties. Such compounds are known to the expert. Their concentration in the media according to the instant invention is preferably 0% to 30%, more preferably 0.1% to 20% and most preferably 1% to 15%.

The response times are given as rise time (τₒₙ) for the time for the change of the relative tuning, respectively of the relative contrast for the electro-optical response, from 0% to 90% (t₉₀-t₀), i.e. including the delay time (t₁₀-t₀), as decay time (τ_{off}) for the time for the change of the relative tuning, respectively of the relative contrast for the electro-optical response, from 100% back to 10% (t₁₀₀-t₁₀) and as the total response time (τₜₒₜₐₗ=τₒₙ+τ_{off}), respectively.

The liquid-crystal media according to the invention consist of a plurality of compounds, preferably 3 to 30, more preferably 4 to 20 and very preferably 4 to 16 compounds. These compounds are mixed in a conventional manner. In general, the desired amount of the compound used in the smaller amount is dissolved in the compound used in the larger amount. If the temperature is above the clearing point of the compound used in the higher concentration, it is particularly easy to observe completion of the dissolution process. It is, however, also possible to prepare the media in other conventional ways, for example using so-called pre-mixes, which can be, for example, homologous or eutectic mixtures of compounds, or using so-called “multibottle” systems, the constituents of which are themselves ready-to-use mixtures.

All temperatures, such as, for example, the melting point T(C,N) or T(C,S), the transition from the smectic (S) to the nematic (N) phase T(S,N) and the clearing point T(N,I) of the liquid crystals, are quoted in degrees Celsius. All temperature differences are quoted in differential degrees.

In the present invention and especially in the following examples, the structures of the mesogenic compounds are indicated by means of abbreviations, also referred to as acronyms. In these acronyms, the chemical formulae are abbreviated as follows using Tables A to D below. All groups CₙH₂ₙ₊₁) CₘH₂ₘ₊₁ and CₗH₂ₗ₊₁ or CₙH₂ₙ, CₘH₂ₘ and CₗH₂ₗ denote straight-chain alkyl or alkylene, where n, m and l are 1, 2, 3, 4, 5, 6 or 7. Table A lists the codes used for the ring elements of the core structures of the compounds, while Table B shows the linking groups. Table C gives the meanings of the codes for the left-hand or right-hand end groups. Table D shows illustrative structures of compounds with their respective abbreviations.

TABLE A
Ring elements

| | |
|---|---|
| 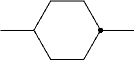 | C |
| 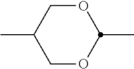 | D |
| 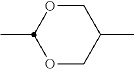 | DI |
| 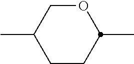 | A |
| 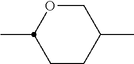 | AI |
| 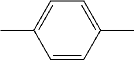 | P |
| 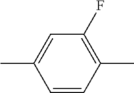 | G |
| 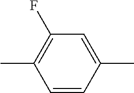 | GI |
| 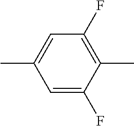 | U |
| 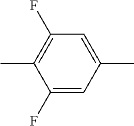 | UI |
| 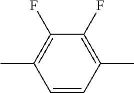 | Y |
| 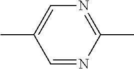 | M |
| 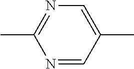 | MI |
| 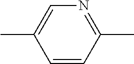 | N |
| 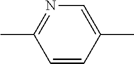 | NI |
| 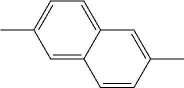 | Np |
| 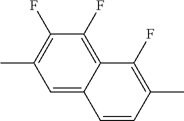 | N3f |
| 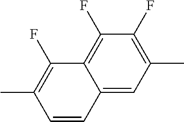 | N3fI |
| 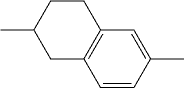 | tH |
| 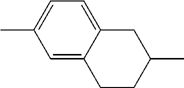 | tHI |
| 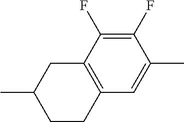 | tH2F |
| 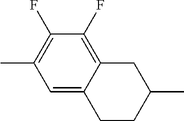 | tH2FI |
| 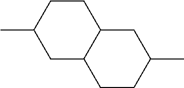 | dH |
| 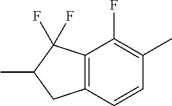 | K |
| 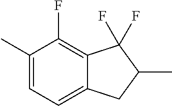 | KI |
| 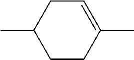 | L |
| 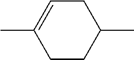 | LI |
| 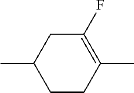 | F |
| 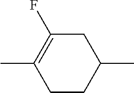 | FI |
| 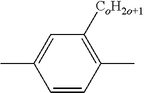 | P(o) |
| 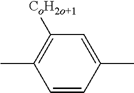 | PI(o) |
| 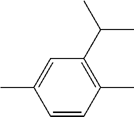 | P(i3) |
| 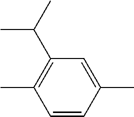 | PI(ic3) |
| 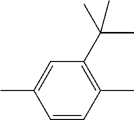 | P(t4) |
| 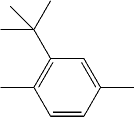 | PI(t4) |
| 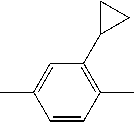 | P(c3) |
| 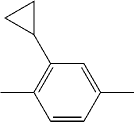 | PI(c3) |
| 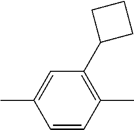 | P(c4) |
| 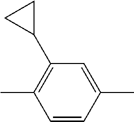 | PI(c4) |
| 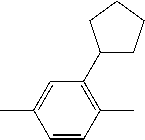 | P(c5) |
| 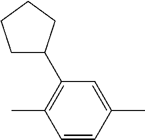 | PI(c5) |
| 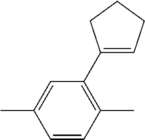 | P(e5) |
| 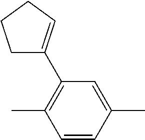 | PI(e5) |
| 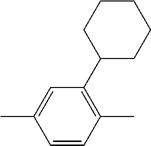 | P(c6) |
| 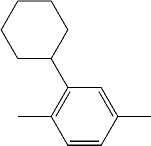 | PI(c6) |
| 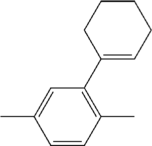 | P(e6) |
| 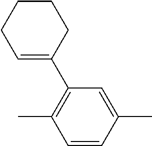 | PI(e6) |
| 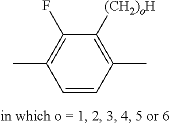 | GI(o) |
| 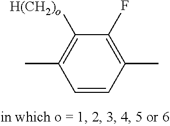 | G(o) |
| 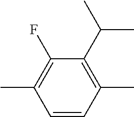 | GI(i3) |
| 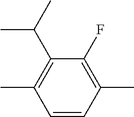 | G(i3) |
| 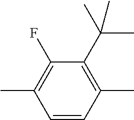 | GI(t4) |
| 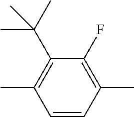 | G(t4) |
| 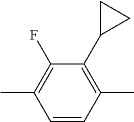 | GI(c3) |
| 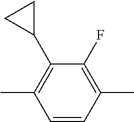 | G(c3) |
| 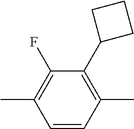 | GI(c4) |
| 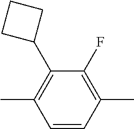 | G(c4) |
| 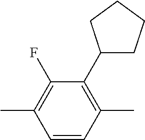 | GI(c5) |
| 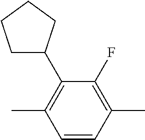 | G(c5) |
| 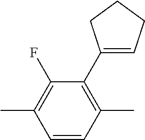 | GI(e5) |
| 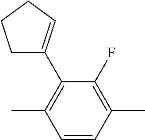 | G(e5) |
| 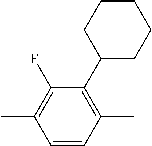 | GI(c6) |
| 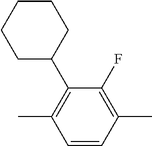 | G(c6) |
| 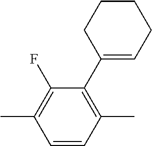 | GI(e6) |
| 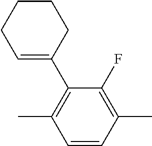 | G(e6) |
| 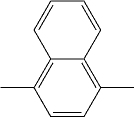 | NP(1,4) |
| 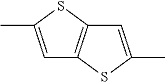 | Th |

TABLE B
Linking groups

| | | | |
|---|---|---|---|
| E | —CH₂CH₂— | Z | —CO—O— |
| V | —CH═CH— | ZI | —O—CO— |
| X | —CF═CH— | O | —CH₂—O— |
| XI | —CH═CF— | OI | —O—CH₂— |
| B | —CF═CF— | Q | —CF₂—O— |
| T | —C≡C— | QI | —O—CF₂— |
| W | —CF₂CF₂— | | |

TABLE B
End groups

| Left-hand side | | Right-hand side | |
|---|---|---|---|
| | | Used alone | |
| -n- | CₙH₂ₙ₊₁— | -n | —CₙH₂ₙ₊₁ |
| -nO- | CₙH₂ₙ₊₁—O— | -On | —O—CₙH₂ₙ₊₁ |
| —V— | CH₂═CH— | -V | —CH═CH₂ |
| -nV- | CₙH₂ₙ₊₁—CH═CH— | -nV | —CₙH2ₙ—CH═CH₂ |
| -Vn- | CH₂═CH—CₙH₂ₙ₊₁— | -Vn | —CH═CH—CₙH₂ₙ₊₁ |
| -nVm- | CₙH₂ₙ₊₁—CH═CH—CₘH₂ₘ— | -nVm | —CₙH₂ₙ—CH═CH—CmH₂ₙ₁₊₁ |
| —N— | N≡C— | —N | —C≡N |
| —S— | S═C═N— | —S | —N═C═S |
| —F— | F— | —F | —F |
| -CL- | Cl— | -CL | —Cl |
| -M- | CFH₂— | -M | —CFH₂ |
| -D- | CF₂H— | -D | —CF₂H |
| -T- | CF₃— | -T | —CF₃ |
| -MO- | CFH₂O— | -OM | —OCFH₂ |
| -DO- | CF₂HO— | -OD | —OCF₂H |
| -TO- | CF₃O— | -OT | —OCF₃ |
| -FXO- | CF₂═CH—O— | -OXF | —O—CH═CF₂ |
| -A- | H—C≡C— | -A | —C≡C—H |
| -nA- | CₙH₂ₙ₊₁—C≡C— | -An | —C≡C—CₙH₂ₙ₊₁ |
| -NA- | N≡C—C≡C— | -AN | —C≡C—C≡N |

Used in combination with others

| | | | |
|---|---|---|---|
| - . . . A . . . - | —C≡— | - . . . A . . . | —C≡— |
| - . . . V . . . - | —CH═CH— | - . . . V . . . | —CH═CH— |
| - . . . Z . . . - | —CO—O— | - . . . Z . . . | —CO—O— |
| - . . . ZI . . . - | —CO— | - . . . ZI . . . | —O—CO— |
| - . . . K . . . - | —CO— | - . . . K . . . | —CO— |
| - . . . W . . . - | —CF═CF— | - . . . W . . . | —CF═CF— |

in which n and m each denote integers, and the three dots “ . . . ” are place-holders for other abbreviations from this table.

The following table shows illustrative structures together with their respective abbreviations. These are shown in order to illustrate the meaning of the rules for the abbreviations. They furthermore represent compounds which are preferably used.

TABLE C
Illustrative structures
The following illustrative structures are compounds, which are preferably
additionally used in the media:

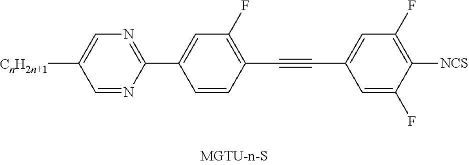

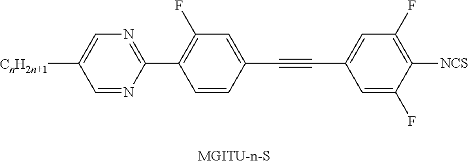

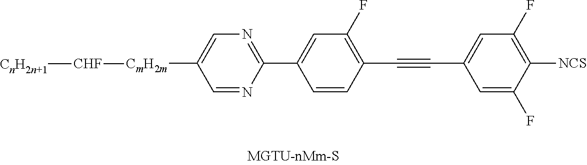

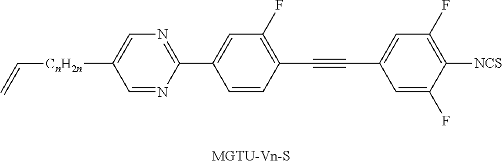

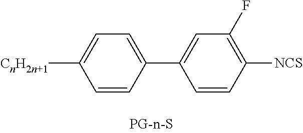

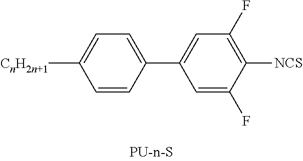

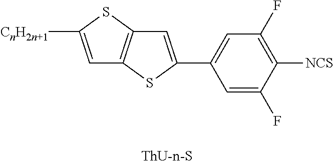

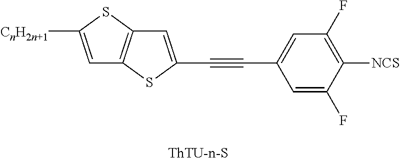

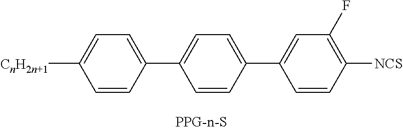

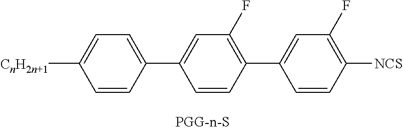

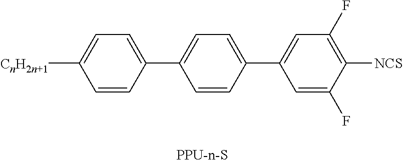

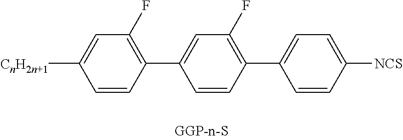

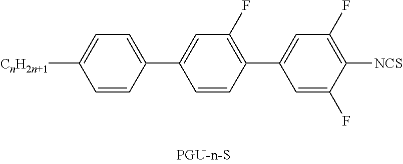

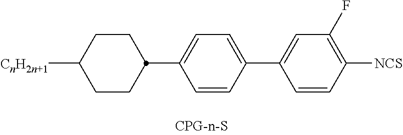

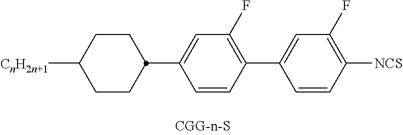

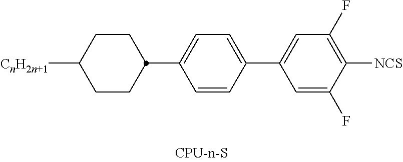

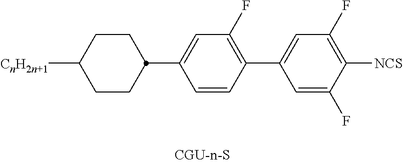

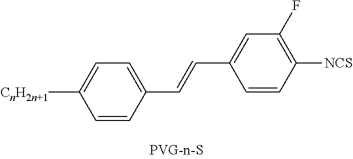

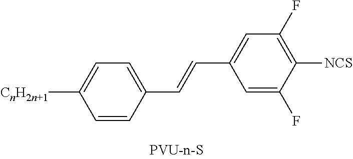

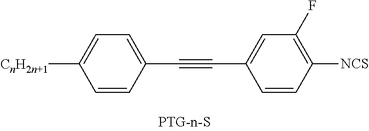

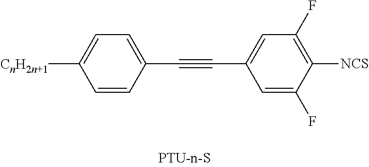

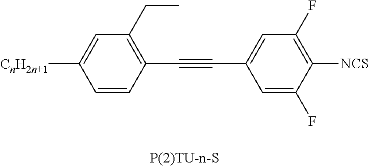

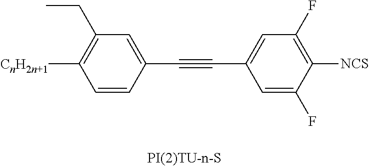

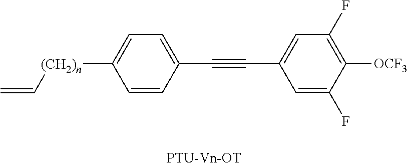

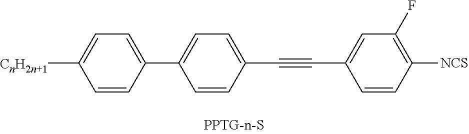

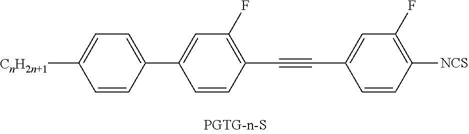

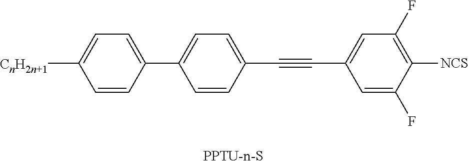

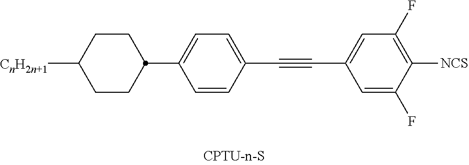

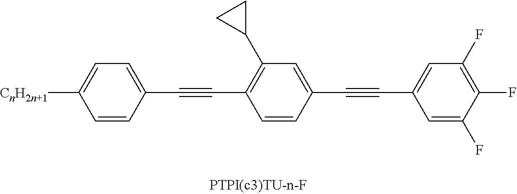

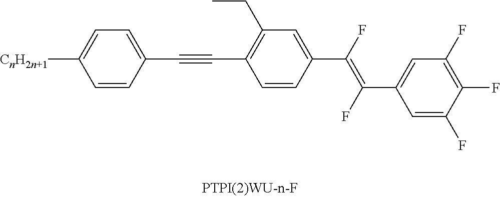

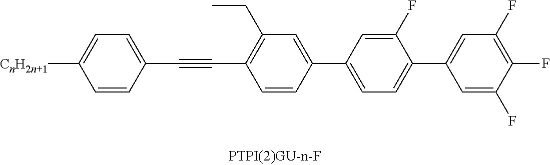

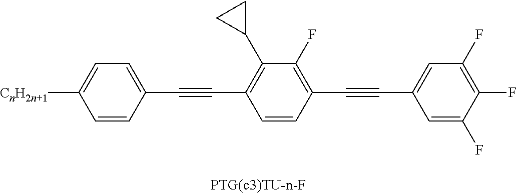

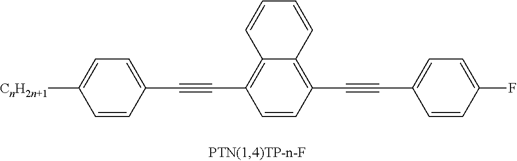

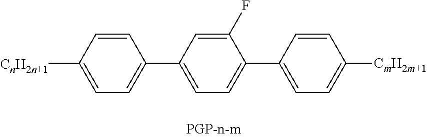

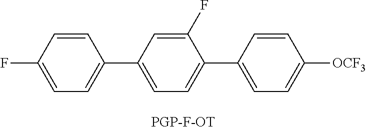 [SMILES: Fc1ccc(-c2ccc(-c3ccc(OC(F)(F)F)cc3)c(F)c2)cc1]

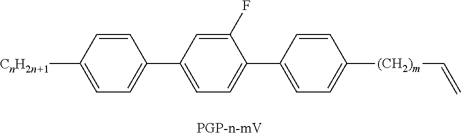

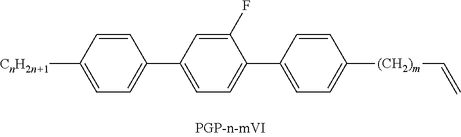

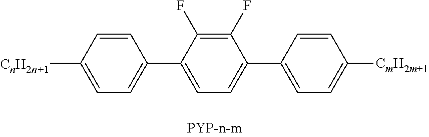

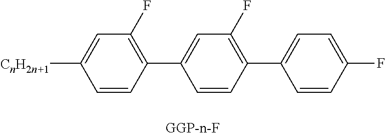

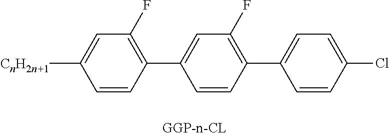

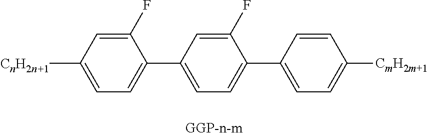

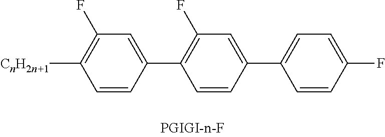

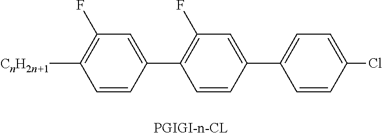

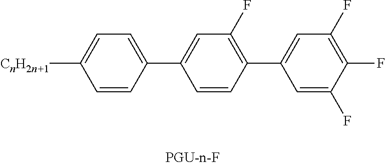

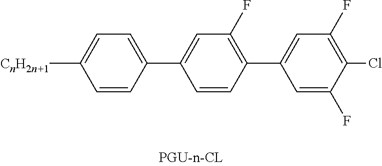

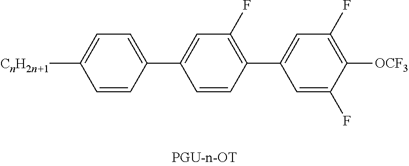

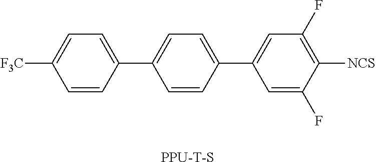

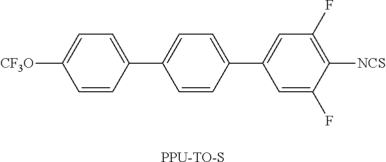

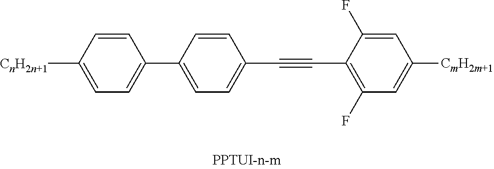

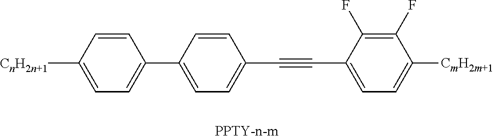

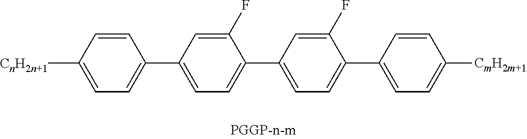

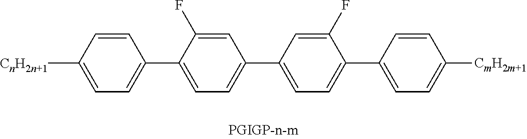

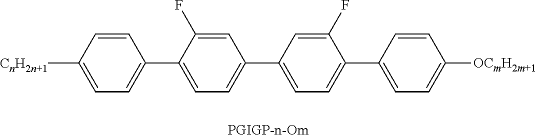

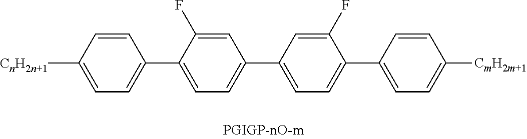

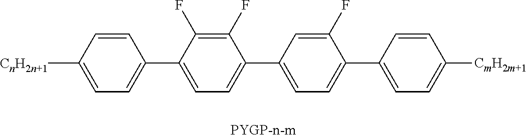

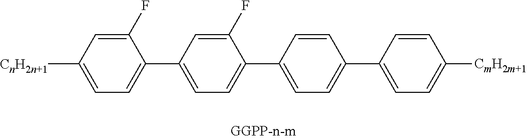

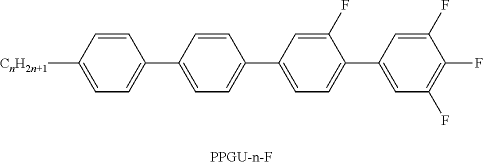

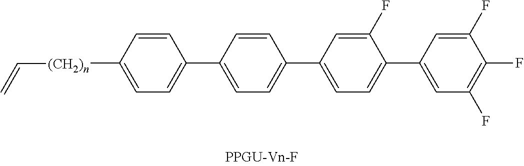

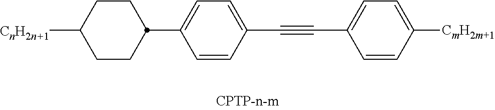

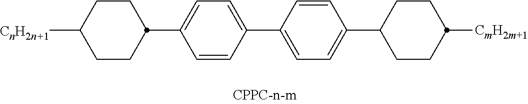

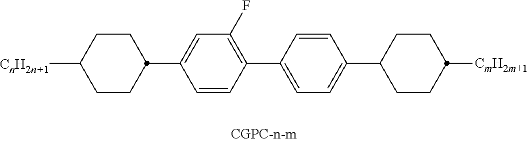

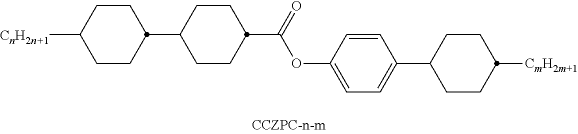

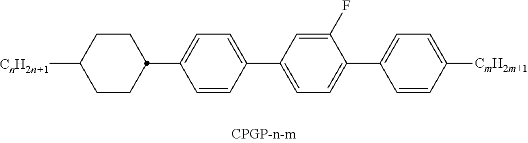

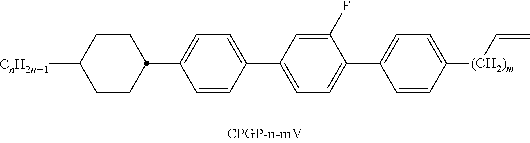

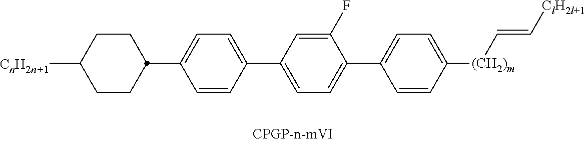

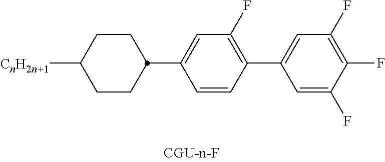

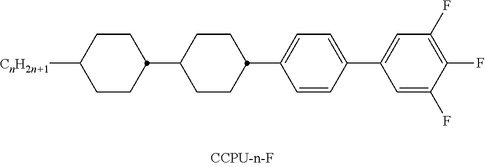

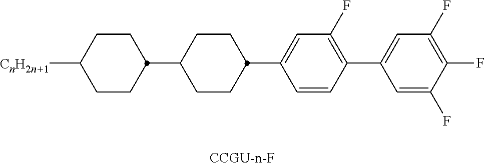

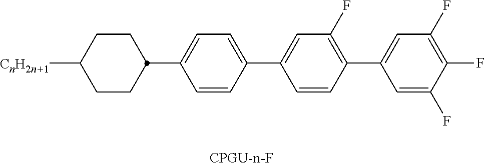

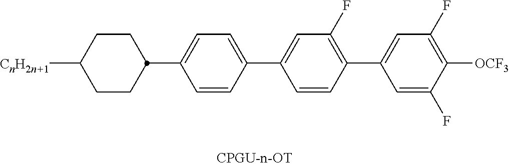

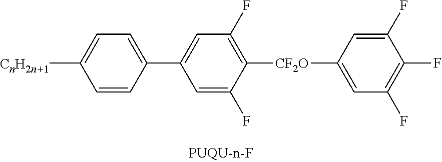

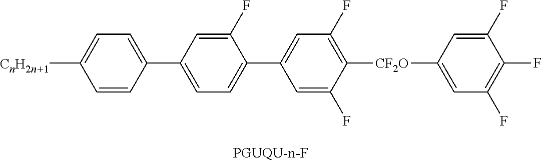

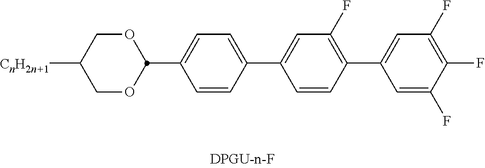

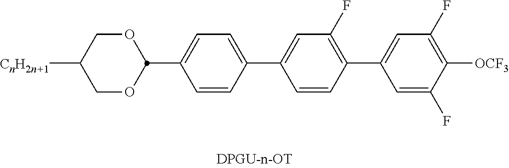

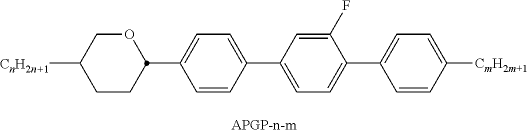

The following table, Table E, shows illustrative compounds which can be used as stabiliser in the mesogenic media in accordance with the present invention. The total concentration of these and similar compounds in the media is preferably 5% or less.

TABLE E

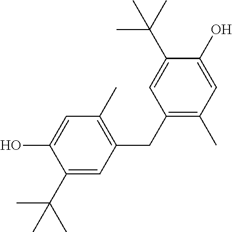 [SMILES: Cc1cc(O)c(C(C)(C)C)cc1Cc1cc(C(C)(C)C)c(O)cc1C]

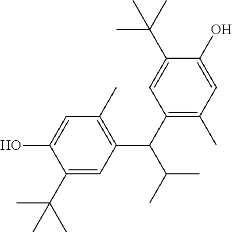 [SMILES: Cc1cc(O)c(C(C)(C)C)cc1C(c1cc(C(C)(C)C)c(O)cc1C)C(C)C]

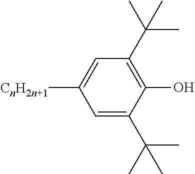

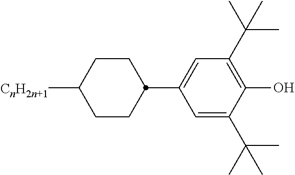

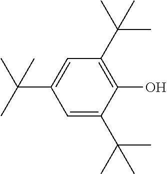 [SMILES: CC(C)(C)c1cc(C(C)(C)C)c(O)c(C(C)(C)C)c1]

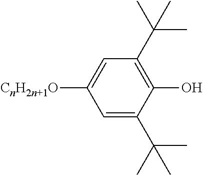

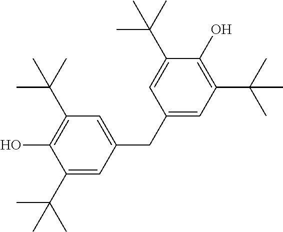 [SMILES: CC(C)(C)c1cc(Cc2cc(C(C)(C)C)c(O)c(C(C)(C)C)c2)cc(C(C)(C)C)c1O]

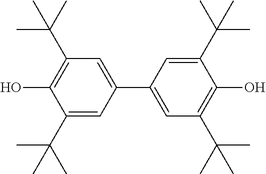 [SMILES: CC(C)(C)c1cc(-c2cc(C(C)(C)C)c(O)c(C(C)(C)C)c2)cc(C(C)(C)C)c1O]

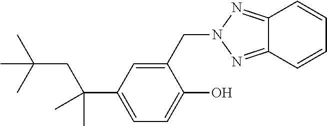 [SMILES: CC(C)(C)CC(C)(C)c1ccc(O)c(Cn2nc3ccccc3n2)c1]

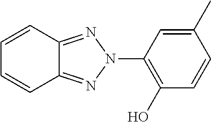 [SMILES: Cc1ccc(O)c(-n2nc3ccccc3n2)c1]

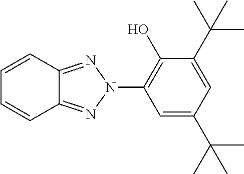 [SMILES: CC(C)(C)c1cc(-n2nc3ccccc3n2)c(O)c(C(C)(C)C)c1]

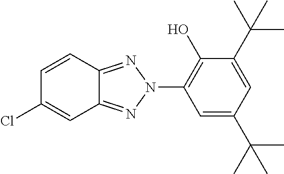 [SMILES: CC(C)(C)c1cc(-n2nc3ccc(Cl)cc3n2)c(O)c(C(C)(C)C)c1]

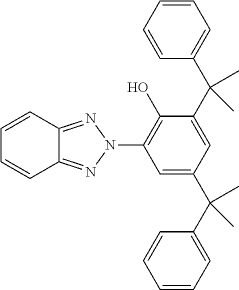 [SMILES: CC(C)(c1ccccc1)c1cc(-n2nc3ccccc3n2)c(O)c(C(C)(C)c2ccccc2)c1]

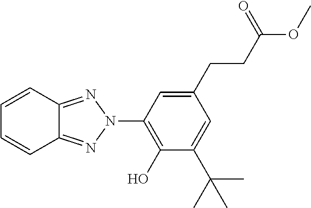 [SMILES: COC(=O)CCc1cc(-n2nc3ccccc3n2)c(O)c(C(C)(C)C)c1]

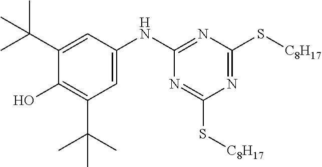

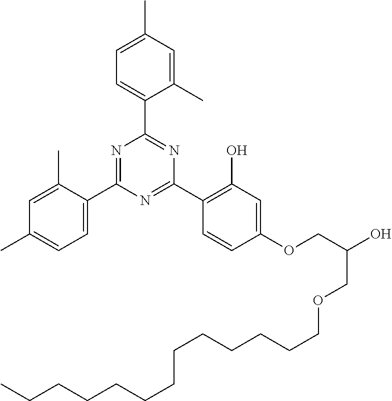 [SMILES: CCCCCCCCCCCCCOCC(O)COc1ccc(-c2nc(-c3ccc(C)cc3C)nc(-c3ccc(C)cc3C)n2)c(O)c1]

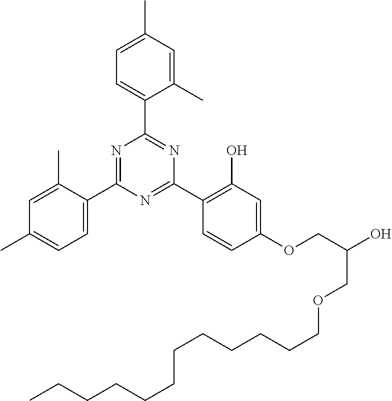 [SMILES: CCCCCCCCCCCCOCC(O)COc1ccc(-c2nc(-c3ccc(C)cc3C)nc(-c3ccc(C)cc3C)n2)c(O)c1]

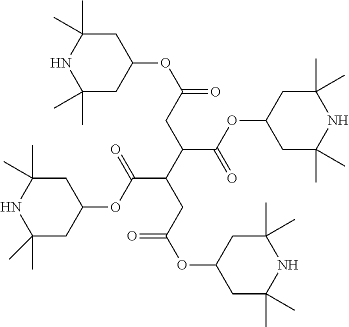

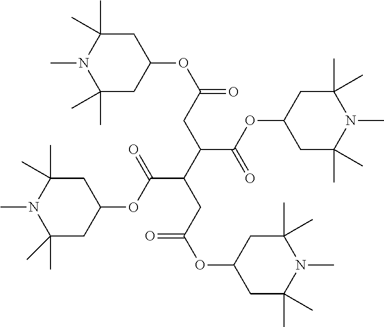

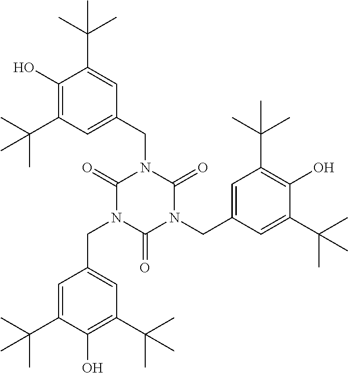 [SMILES: CC(C)(C)c1cc(Cn2c(=O)n(Cc3cc(C(C)(C)C)c(O)c(C(C)(C)C)c3)c(=O)n(Cc3cc(C(C)(C)C)c(O)c(C(C)(C)C)c3)c2=O)cc(C(C)(C)C)c1O]

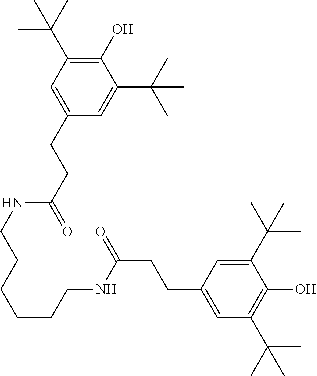 [SMILES: CC(C)(C)c1cc(CCC(=O)NCCCCCCNC(=O)CCc2cc(C(C)(C)C)c(O)c(C(C)(C)C)c2)cc(C(C)(C)C)c1O]

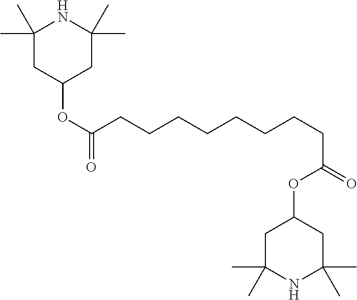

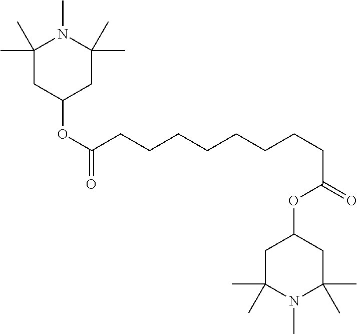

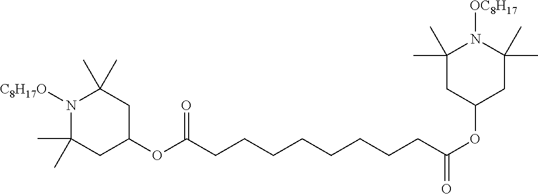

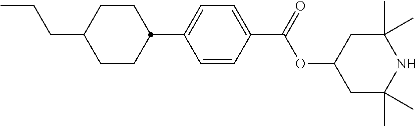

In a preferred embodiment of the present invention, the mesogenic media comprise one or more compounds selected from the group of the compounds from Table E.

The following table, Table F, shows illustrative compounds which can preferably be used as chiral dopants in the mesogenic media in accordance with the present invention.

TABLE F

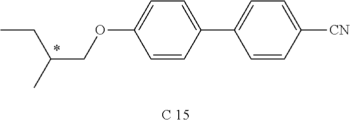 [SMILES: CCC(C)COc1ccc(-c2ccc(C#N)cc2)cc1]

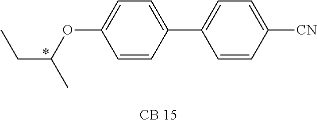 [SMILES: CCC(C)Oc1ccc(-c2ccc(C#N)cc2)cc1]

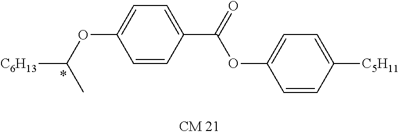

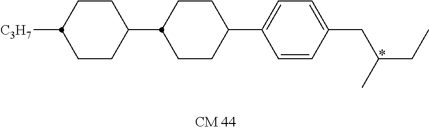

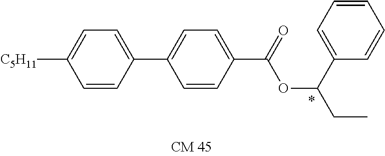

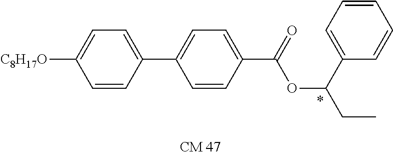

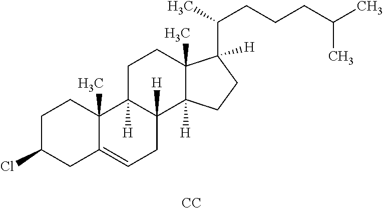

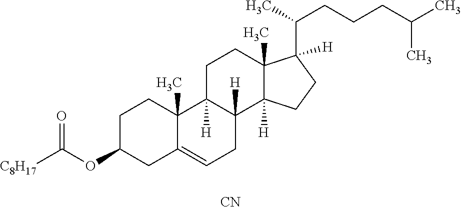

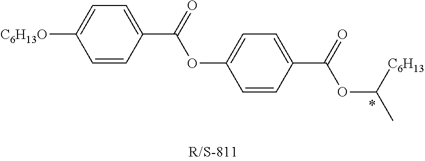

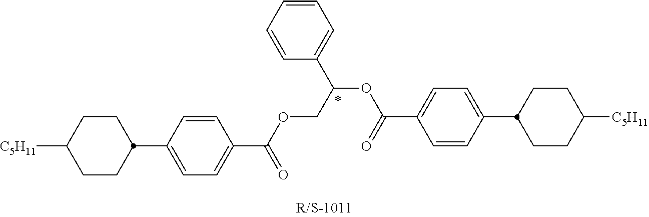

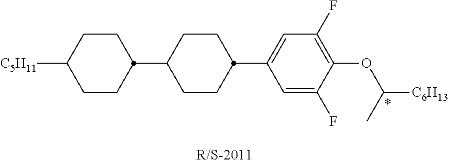

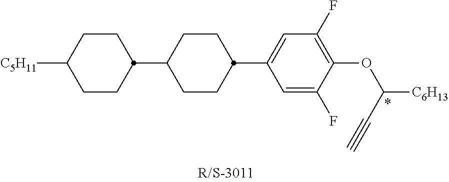

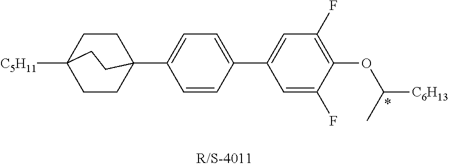

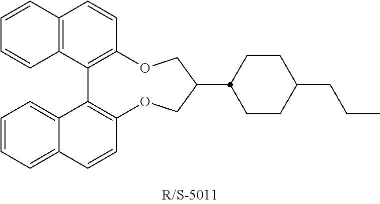

In a preferred embodiment of the present invention, the mesogenic media comprise one or more compounds selected from the group of the compounds of Table F.

The mesogenic media in accordance with the present application preferably comprise two or more, preferably four or more, compounds selected from the group consisting of the compounds from the above tables.

The liquid-crystal media in accordance with the present invention preferably comprise seven or more, preferably eight or more, compounds, preferably compounds having three or more, preferably four or more, different formulae, selected from the group of the compounds from Table D.

### EXAMPLES

The following examples illustrate the present invention without limiting it in any way.

However, it is clear to the person skilled in the art from the physical properties what properties can be achieved and in what ranges they can be modified. In particular, the combination of the various properties which can preferably be achieved is thus well defined for the person skilled in the art.

### Mixture Examples

Liquid-crystal host mixture H1 having the compositions and properties as indicated in the following tables are prepared and characterized with respect to their general physical properties and their applicability in microwave components at 19 GHz and 20° C.

Mixture H1

| | | | |
|---|---|---|---|
| BCH-3F.F | 12.0% | T(N, I) | = 92° C. |
| BCH-5F.F | 10.0% | Δn (20° C., 589.3 nm) | = 0.0969 |
| ECCP-30CF3 | 5.0% | Δϵ (20° C., 1 kHz) | = 5.2 |
| ECCP-50CF3 | 5.0% | | |
| CBC-33F | 2.0% | | |
| CBC-53F | 2.0% | | |
| CBC-55F | 2.0% | | |
| PCH-6F | 8.0% | | |
| PCH-7F | 6.0% | | |
| CCP-20CF3 | 8.0% | | |
| CCP-30CF3 | 12.0% | | |
| CCP-40CF3 | 7.0% | | |
| CCP-50CF3 | 11.0% | | |
| PCH-5F | 10.0% | | |
| Σ | 100.0% | | |

A nematic liquid-crystal medium N1 consisting of 90% of the medium H1 and 10% of the compound of Synthesis Example 1 (compound (1)) has the following properties:

| | | | | |
|---|---|---|---|---|
| N1 | 90.0% | T(N, I) | = 93 | ° C. |
| (1) | 10.0% | Δn (20° C., 589.3 nm) | = 0.1341 | |
| Σ | 100.0% | Δϵ (20° C., 1 kHz) | = 6.7 | |

The compound (1) is well soluble in the medium H1. The addition of the compound (1) to the medium H1 has no negative influence in the properties of the host H1.

The addition of the compound (1) to the medium H1 has the effect that the material quality η is significantly improved due to a higher tunability (τ) and lower dielectric loss (tan δ_{εr,⊥}) of the medium, as shown in the following table.

| | εᵣ,_{\|\|} | tan δ_{ε}_{_{r,\|\|}} | ε_{r,⊥} | tan δ_{ε}_{_{r,⊥}} | τ | η |
|---|---|---|---|---|---|---|
| H1 | 2.4873 | 0.0045 | 2.2327 | 0.0123 | 0.1024 | 8.3 |
| N1 | 2.6315 | 0.0049 | 2.2902 | 0.0121 | 0.1297 | 10.7 |

Further mixtures are prepared and charactersied as follows:

Mixture N2

| | | | |
|---|---|---|---|
| PTU-3-S | 10.0% | T_{(N, I)}. [° C.]: | 140.0 |
| PTU-5-S | 8.0% | Δn (589.3 nm, 20° C.): | 0.3777 |
| PGU-3-S | 18.0% | Δϵ (1 kHz, 20° C.): | 21.8 |
| PPTU-4-S | 7.0% | | |
| PPTU-5-S | 9.0% | | |
| PGTU-4-S | 6.0% | | |
| CPU-2-S | 22.0% | | |
| CPU-4-S | 10.0% | | |
| ThTU-5-S | 10.0% | | |
| Σ | 100.0% | | |

Mixture N3

| | | | |
|---|---|---|---|
| PTU-3-S | 8.0% | T_{(N, I)}. [° C.]: | 148.5 |
| PG U-3-S | 18.0% | Δn (589.3 nm, 20° C.): | 0.3801 |
| PPTU-4-S | 7.0% | Δϵ (1 kHz, 20° C.): | 21.1 |
| PPTU-5-S | 9.0% | | |
| PGTU-4-S | 6.0% | | |
| CPU-2-S | 22.0% | | |
| CPU-4-S | 10.0% | | |
| ThTU-5-S | 10.0% | | |
| ThU-5-S | 10.0% | | |
| Σ | 100.0% | | |

Mixture N4

| | | | |
|---|---|---|---|
| PGU-3-S | 18.0% | T_{(N, I)}. [° C.]: | 151.0 |
| PPTU-4-S | 7.0% | Δn (589.3 nm, 20° C.): | 0.3900 |
| PPTU-5-S | 9.0% | ΔE (1 kHz, 20° C.): | 21.1 |
| PGTU-4-S | 6.0% | | |
| CPU-2-S | 22.0% | | |
| CPU-4-S | 6.0% | | |
| ThTU-5-S | 16.0% | | |
| ThU-5-S | 16.0% | | |
| Σ | 100.0% | | |

1. A Liquid-crystal medium, characterised in that the medium comprises one or more compounds of formula S 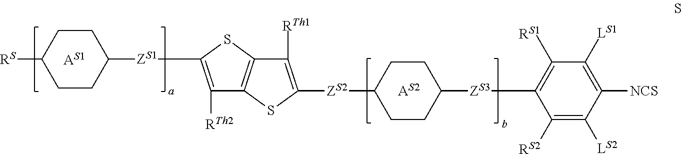
in which
R^{S} denotes H, alkyl or alkoxy having 1 to 12 C atoms, or alkenyl, alkenyloxy or alkoxyalkyl having 2 to 12 C atoms, in which one or more CH₂-groups may be replaced by 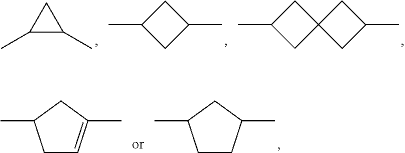
and
in which one or more H atoms may be replaced by F, 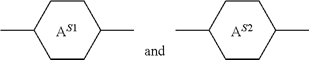
on each occurrence, independently of one another, denote 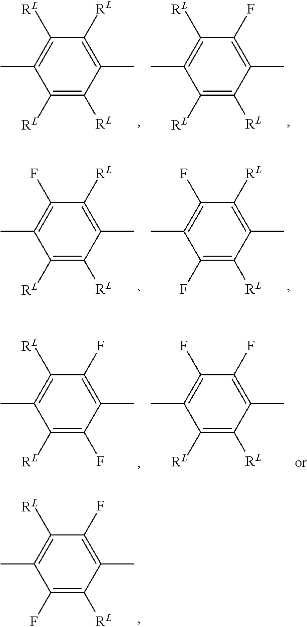
in which R^{L}, on each occurrence identically or differently, denotes H, Cl or alkyl having 1 to 6 C atoms, 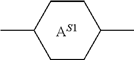
alternatively denotes 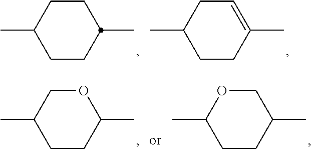
L^{S1}, L^{S2} identically or differently, denote H, Cl or F,
R^{S1}, R^{S2}, identically or differently, denote H, alkyl or alkenyl, having up to 6 C atoms, or cyclopropyl, cyclobutyl, cyclopentenyl, or cyclopentyl,
R^{Th1}, R^{Th2} identically or differently, denote H, alkyl or alkenyl or alkoxy, having up to 6 C atoms, or cyclopropyl, cyclobutyl, cyclopentenyl or cyclopentyl,
Z^{S1}, Z^{S2}, Z^{S3} identically or differently, denote —CH═CH—, —CH═CF—, —CF═CH—, —CF═CF—, —C≡C—, or a single bond,
a, b identically or differently, are 0 or 1,
and
one or more compounds of formula IC 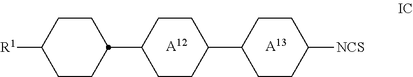
in which
R¹ denotes H, alkyl or alkoxy having 1 to 15 C atoms, or alkenyl, alkenyloxy or alkoxyalkyl having 2 to 15 C atoms, in which one or more CH₂-groups may be replaced by 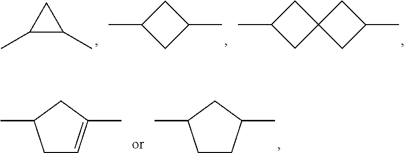 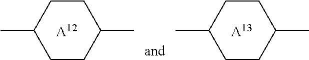
on each occurrence, independently of one another, denote 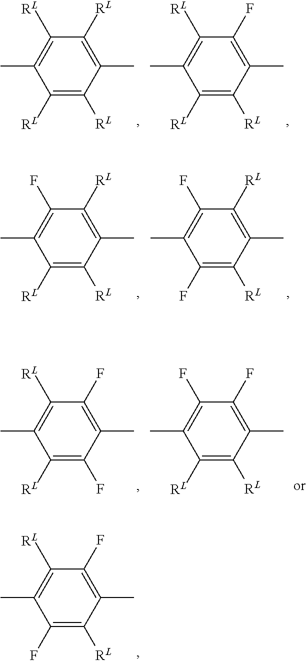
in which R^{L}, on each occurrence identically or differently, denotes H or alkyl having 1 to 6 C atoms.

2. The medium according to claim 1, wherein the medium has a clearing temperature of 120° C. or more.

3. The medium according to claim 1, wherein the medium comprises one or more compounds selected from the group of compounds of the formulae S1 to S24 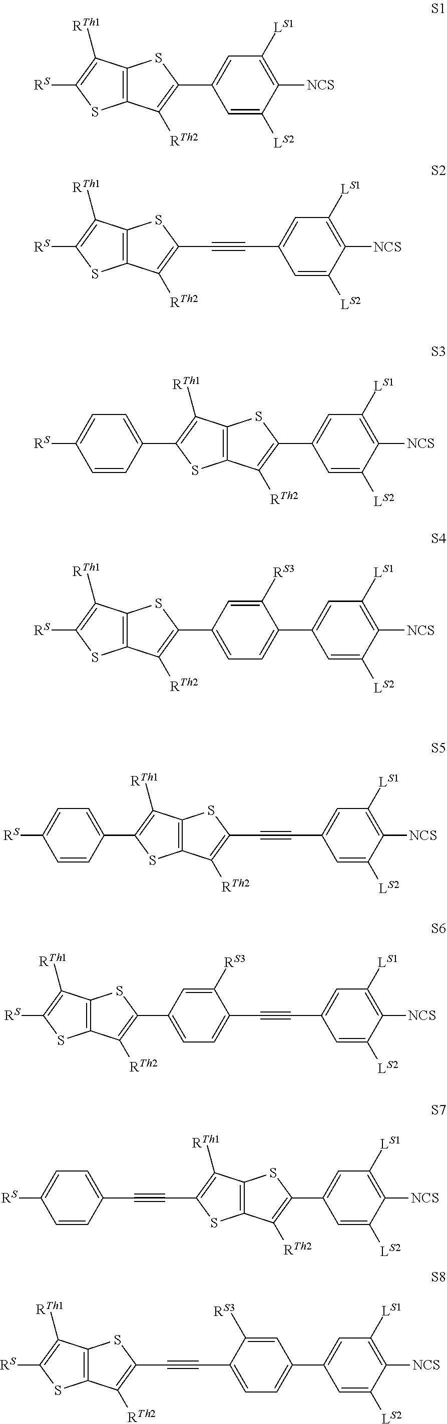 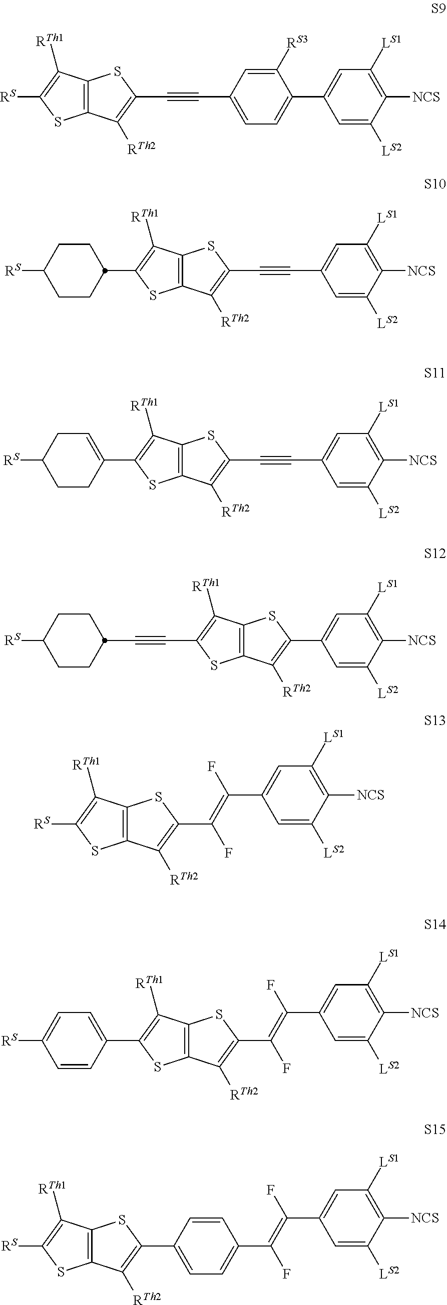 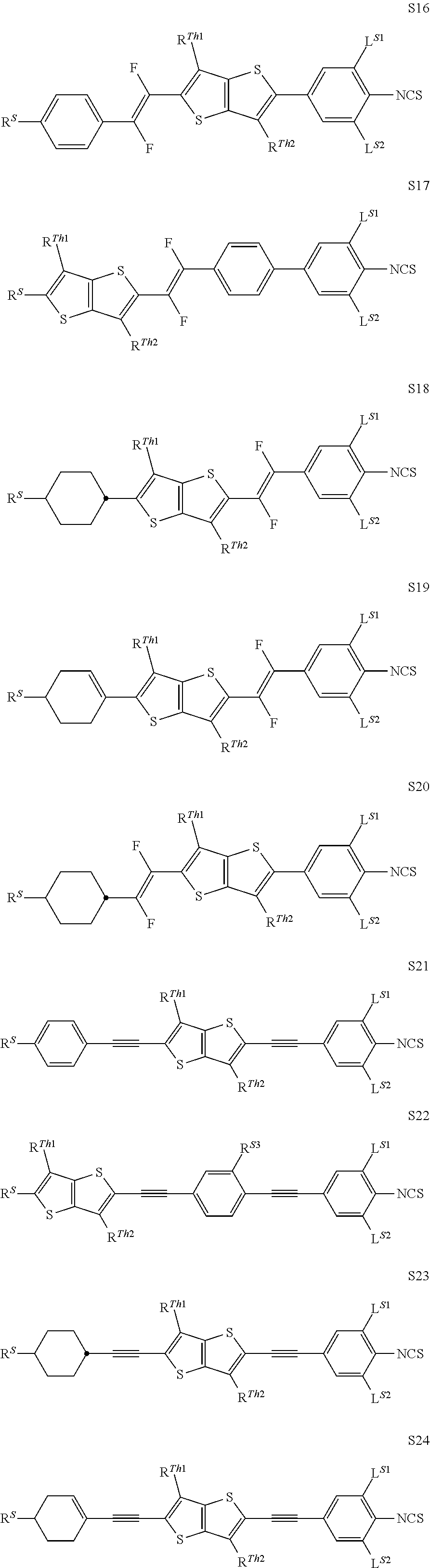
in which R^{S}, R^{Th1}, R^{Th2}, L^{S1} and L^{S2} have the meanings indicated in claim 1 for formula S and
R^{S3} denotes H, F or alkyl, having up to 6 C atoms, or cyclopropyl.

4. The medium according to claim 1, wherein the medium comprises a compound of formula S in which at least one of the groups Z^{S1}, Z^{S2} and Z^{S3} denotes —C≡C—.

5. The liquid-crystal medium according to claim 1, wherein the medium further comprises one or more compounds selected from the group of compounds of formulae I, II and III 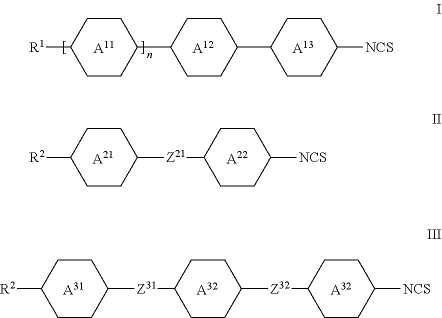
in which
R¹ denotes H, alkyl or alkoxy having 1 to 15 C atoms, or alkenyl, alkenyloxy or alkoxyalkyl having 2 to 15 C atoms, in which one or more CH₂-groups may be replaced by 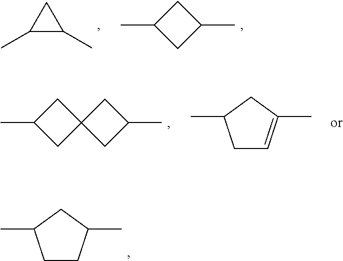
n is 0, 1 or 2, 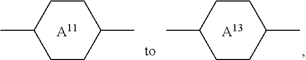
on each occurrence, independently of one another, denote 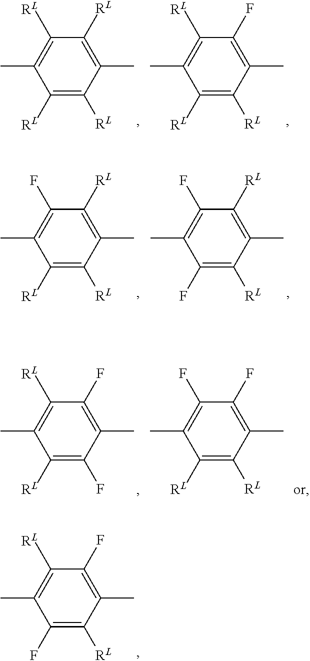
in which R^{L}, on each occurrence identically or differently, denotes H or alkyl having 1 to 6 C atoms,
and wherein 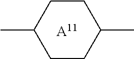
alternatively denotes 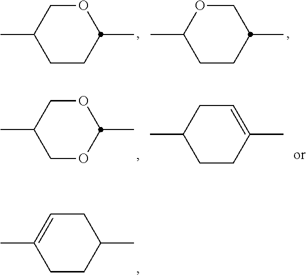
R² denotes H, alkyl or alkoxy having 1 to 15 C atoms, or alkenyl, alkenyloxy or alkoxyalkyl having 2 to 15 C atoms, in which one or more CH₂-groups may be replaced by 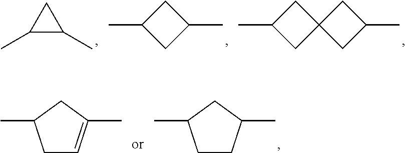
Z²¹ denotes trans-CH═CH—, trans-CF═CF— or —C≡C—, and 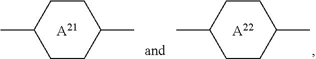
independently of one another, denote 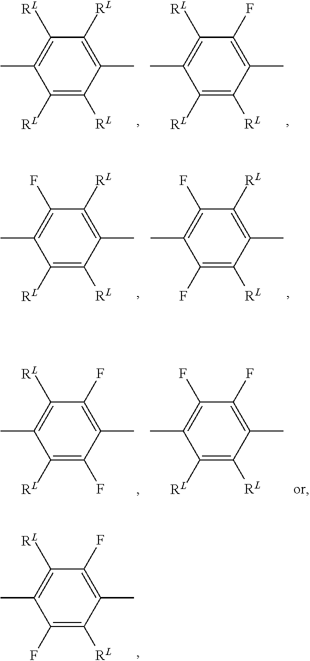
in which R^{L}, on each occurrence identically or differently, denotes H or alkyl having 1 to 6 C atoms,
R³ denotes H, alkyl or alkoxy having 1 to 15 C atoms, or alkenyl, alkenyloxy or alkoxyalkyl having 2 to 15 C atoms, in which one or more CH₂-groups may be replaced by 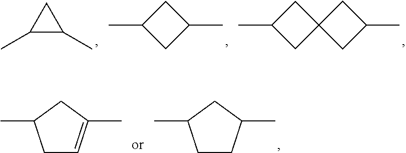
one of Z³¹ and Z³², denotes trans-CH═CH—, trans-CF═CF— or —C≡C— and the other one, independently thereof, denotes —C≡C—, trans-CH═CH—, trans-CF═CF— or a single bond, and 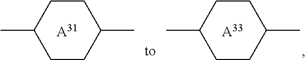
independently of one another, denote 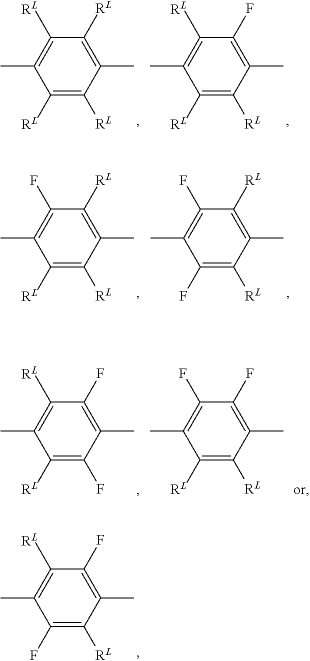
in which R^{L}, on each occurrence identically or differently, denotes H or alkyl having 1 to 6 C atoms,
and wherein 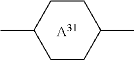
alternatively denotes

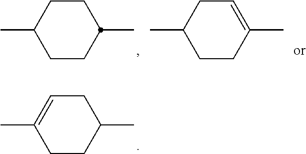

6. The liquid-crystal medium according to claim 1, wherein the medium further comprises one or more compounds of formula IIIC 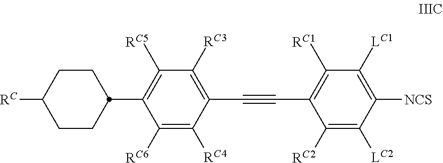
in which
R^{C} denotes H, alkyl or alkoxy having 1 to 12 C atoms, or alkenyl, alkenyloxy or alkoxyalkyl having 2 to 12 C atoms, in which one or more CH₂-groups may be replaced by 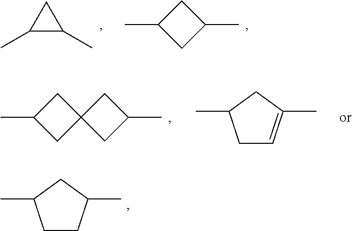
L^{C1}, L^{C2} identically or differently, denote H, Cl or F,
R^{C1}, R^{C2} identically or differently, denote H or alkyl having 1 to 6 C atoms, or cyclopropyl, cyclobutyl or cyclopentyl,
R^{C3}, R^{C4},
R^{C5} and R^{C6} identically or differently, denote H, Cl, F or alkyl having 1 to 6 C atoms, or cyclopropyl, cyclobutyl or cyclopentyl.

7. The liquid-crystal medium according to claim 1, wherein the medium further comprises one or more compounds of formula T 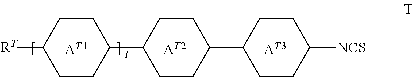
in which
R^{T} denotes halogen, CN, NCS, R^{F}, R^{F}—O— or R^{F}—S—, wherein R^{F} denotes fluorinated alkyl or fluorinated alkenyl having up to 12 C atoms, 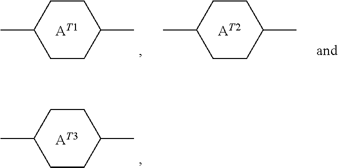
on each occurrence, independently of one another, denote 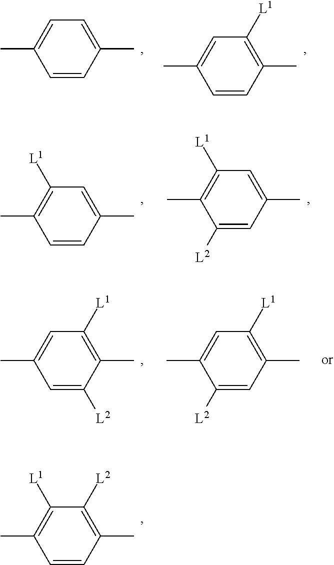
L¹ and L² identically or differently, denote Cl, F, alkyl having 1 to 6 C atoms or cyclopropyl, cyclobutyl or cyclopentyl, and
t is 0, 1 or 2.

8. The liquid-crystal medium according to claim 5, wherein the medium comprises one or more compounds selected from the group of compounds of formulae I-1 to I-4 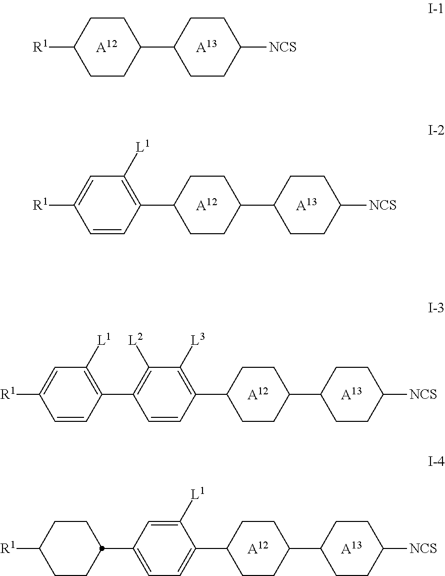
in which
L¹, L² and L³ on each occurrence, identically or differently, denote H or F, and
R¹, 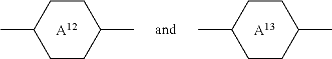
have the meanings given for formula I in claim 5.

9. The liquid-crystal medium according to claim 5, wherein the medium comprises one or more compounds selected from the group of compounds of the formulae II-1 to II-3 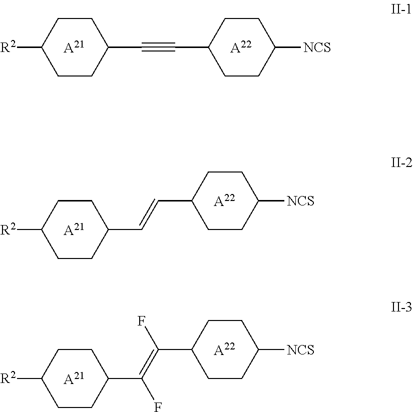
in which
R², 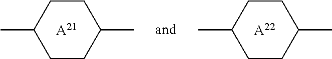
have the meanings given in claim 5 for formula II.

10. The liquid-crystal medium according to claim 5, wherein the medium comprises one or more compounds selected from the group of compounds of the formulae III-1 to III-6 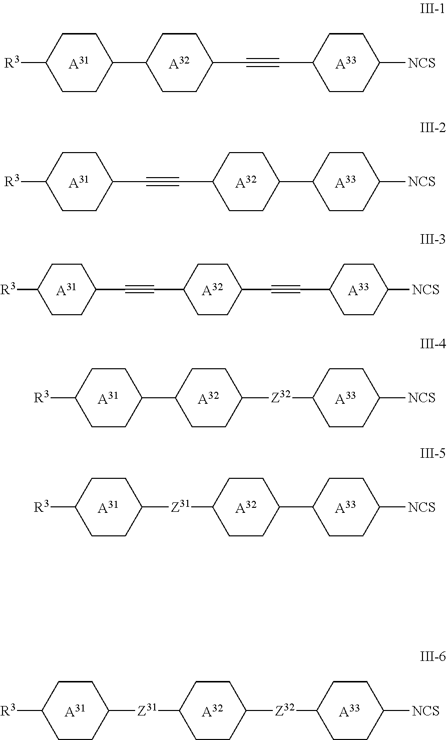
in which
R³, 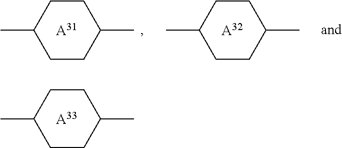
have the meanings given in claim 5 for formula III, and
Z³¹ and Z³² independently of one another, denote trans-CH═CH— or trans-CF═CF—, and in formula III-6 alternatively one of Z³¹ and Z³² denotes —C≡C—.

11. The liquid-crystal medium according to claim 1, wherein the medium further comprises one or more chiral compounds.

12. A compound of formula S 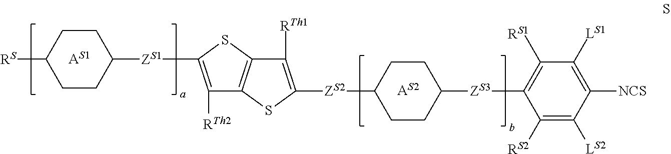
in which
R^{S} denotes H, alkyl or alkoxy having 1 to 12 C atoms, or alkenyl, alkenyloxy or alkoxyalkyl having 2 to 12 C atoms, in which one or more CH₂-groups may be replaced by 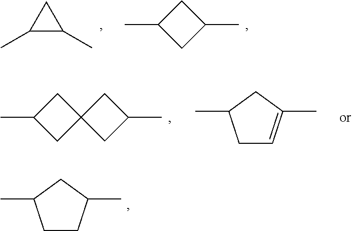
and
in which one or more H atoms may be replaced by F, 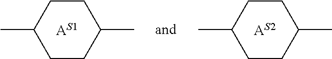
on each occurrence, independently of one another, denote 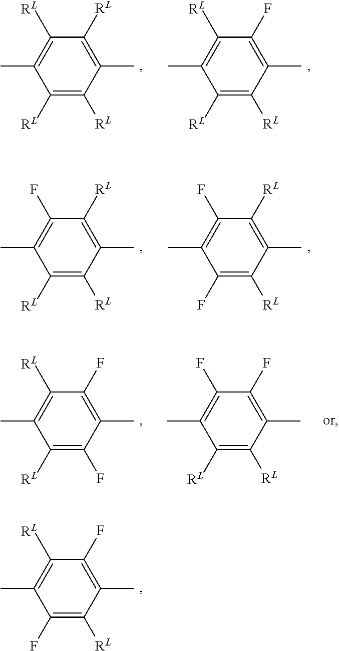
in which R^{L}, on each occurrence identically or differently, denotes H, Cl or alkyl having 1 to 6 C atoms, 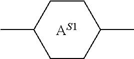
alternatively denotes 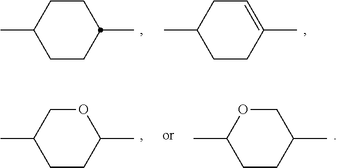
L^{S1}, L^{S2} identically or differently, denote H, Cl or F,
R^{S1}, R^{S2}, identically or differently, denote H or alkyl having 1 to 6 C atoms, or cyclopropyl, cyclobutyl or cyclopentyl,
R^{Th1}, R^{Th2} identically or differently, denote H, alkyl, alkenyl or alkoxy having up to 6 C atoms,
Z^{S1}, Z^{S2}, Z^{S3} identically or differently, denote —CH═CH—, —CH═CF—, —CF═CH—, —CF═CF—, or a single bond,
a, b identically or differently, are 0 or 1,
with the proviso that the compounds of the following formula S* are excluded: 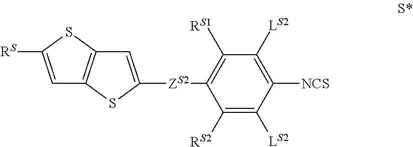
in which
R^{S} denotes alkyl, alkenyl or alkoxy having up to 9 C atoms,
Z^{S2} denotes —CH═CH—, —CH═CF—, —CF═CH—, —CF═CF—, or a single bond,
R^{S1}, R^{S2} identically or differently, denote H or F, and
L^{S1}, L^{S2} identically or differently, denote H or F.

13. Use of a compound of formula S according to claim 12 in a component for high-frequency technology.

14. Component for high-frequency technology comprising a liquid-crystal medium according to claim 1.

15. Component according to claim 14, wherein the component is a liquid-crystal based antenna element, a phase shifter, a tunable filter, a tunable metamaterial structure, a matching network or a varactor.

16. Microwave antenna array comprising one or more components according to claim 14.

17. Process for the preparation of a liquid-crystal medium according to claim 5, comprising mixing one or more compounds of formula S with one or more compounds of formula I and/or II and/or III, and optionally additional mesogenic compounds and optionally a chiral compound.

18. A component for high-frequency technology comprising a compound of formula S according to claim 12.

19. A component according to claim 18, wherein the component is a liquid-crystal based antenna element, a phase shifter, a tunable filter, a tunable metamaterial structure, a matching network or a varactor.

20. A microwave antenna array comprising one or more components according to claim 18.

21. A process for the preparation 1 n of a liquid-crystal medium according to claim 1, comprising mixing one or more compounds of formula S with one or more compounds of one or more compounds of formula IC and optionally additional mesogenic compounds and optionally a chiral compound.
